SEQUENCE LISTING (79 sequences)

SEQ ID NO: 1 - DNA, 13 bp, Artificial Sequence
  Labelling probe - B70_Blong1
ccgacgctgcacc

SEQ ID NO: 2 - DNA, 15 bp, Artificial Sequence
  Labelling probe - B70_Bbrev1
ccgcaagcatccagt

SEQ ID NO: 3 - DNA, 15 bp, Artificial Sequence
  Labelling probe - B194_Bbrev1
gaaggccgtcatcgt

SEQ ID NO: 4 - DNA, 15 bp, Artificial Sequence
  Labelling probe - B70_Bkash1
caacggcctttgtgg

SEQ ID NO: 5 - DNA, 15 bp, Artificial Sequence
  Labelling probe - B194_Badol1
gaaggccaagctggt

SEQ ID NO: 6 - DNA, 13 bp, Artificial Sequence
  Detection probe - B70_Blong1RC-TAM
ggtgcagcgtcgg

SEQ ID NO: 7 - DNA, 15 bp, Artificial Sequence
  Detection probe - B70_Bbrev1RC-TAM
actggatgcttgcgg

SEQ ID NO: 8 - DNA, 18 bp, Artificial Sequence
  Detection probe - B194_Bbrev1RC-TAM
acgatgacggccttcttt

SEQ ID NO: 9 - DNA, 21 bp, Artificial Sequence
  Detection probe - B70_Bkash1RC-TAM
ccacaaaggccgttgtttttt

SEQ ID NO: 10 - DNA, 25 bp, Artificial Sequence
  Detection probe - B194_Badol1RC-TAM
accagcttggccttctttttttttt

SEQ ID NO: 11 - DNA, 15 bp, Artificial Sequence
  Bifidobacteria PCR Primer - B70_2_7F
ggggtgacggcgtgc

SEQ ID NO: 12 - DNA, 21 bp, Artificial Sequence
  Bifidobacteria PCR primer - B70_2_7R
ctcatgtctgcattctcactc

SEQ ID NO: 13 - DNA, 20 bp, Artificial Sequence
  Bifidobacteria PCR primer - B194_15_1F
aacatgcagtggttcaagat

SEQ ID NO: 14 - DNA, 19 bp, Artificial Sequence
  Bifidobacteria PCR primer - B194_15_1R
cggtgatcgggtacttsta

SEQ ID NO: 15 - DNA, 48 bp, Artificial Sequence
  B70_2_7 amplicon adaptor primer - B70_2_7FA
tcgtcggcagcgtcagatgtgtataagagacagggggtgacggcgtgc

SEQ ID NO: 16 - DNA, 55 bp, Artificial Sequence
  B70_2_7 amplicon adaptor primer - B70_2_7RA
gtctcgtgggctcggagatgtgtataagagacagctcatgtctgcattctcactc

SEQ ID NO: 17 - DNA, 53 bp, Artificial Sequence
  B194_15_1 amplicon adaptor primer - B194_15_1FA
tcgtcggcagcgtcagatgtgtataagagacagaacatgcagtggttcaagat

SEQ ID NO: 18 - DNA, 53 bp, Artificial Sequence
  B194_15_1 amplicon adaptor primer - B194_15_1RA
gtctcgtgggctcggagatgtgtataagagacagcggtgatcgggtacttsta

SEQ ID NO: 19 - DNA, 50 bp, Artificial Sequence
  Univ16S amplicon adaptor primer - BHQ2 (black hole quencher 2) SFA
  misc_feature (42)..(42): n is a, c, g, or t
tcgtcggcagcgtcagatgtgtataagagacagcctacgggnggcwgcag

SEQ ID NO: 20 - DNA, 55 bp, Artificial Sequence
  Univ16S amplicon adaptor primer - Univ16SRA
gtctcgtgggctcggagatgtgtataagagacaggactachvgggtatctaatcc

SEQ ID NO: 21 - DNA, 776 bp, Bifidobacterium longum
cacgtcgactgcccgggccacgccgacttcgtgaagaacatgatcaccggtgccgcccagatggatggcgccatcctcgttgtggccgccaccgacggcccgatggcccagacccgcgagcacgtgctgctcgcccgtcaggtcggcgttccgaagatcctcgtcgccctcaacaagtgcgacatggtcgacgatgaagagctcatcgagctcgtcgaagaagaggtccgtgacctcctcgacgagaacggcttcgatcgcgactgcccggtcatccacacctccgcctacggcgccctgcacgacgacgctccggaccacgagaagtgggtccagtccgtcaaggacctcatggccgccgtcgatgactacatcccaaccccggttcacgacctggacaagccgttcctgatgccgatcgaggacgtcttcaccatctccggccgtggtaccgtcgtcaccggtcgtgtcgagcgtggccagctggccgtcaacaccccggtcgagatcgtcggcatccgtccgacccagaccaccaccgtcacctccatcgagaccttccacaagaccatggacgcctgcgaggctggcgacaacaccggtctgctgctgcgtggcctcggccgtgaggatgtcgagcgcggtcaggtcgtggccaagccgggctccgtcaccccgcacaccaagttcgagggcgaagtctacgtgctgaccaaggacgaaggcggccgtcactcgccgttcttctccaactaccgtccgcagttctacttccg

SEQ ID NO: 22 - DNA, 776 bp, Bifidobacterium adolescentis
cacgtcgactgcccgggccacgccgacttcgtgaagaacatgatcaccggcgccgctcagatggatggcgccatccttgttgtggccgccaccgacggcccgatggctcagacccgcgagcacgtgctgctcgctcgtcaggtgggcgtcccgaagatcctcgtcgctctgaacaagtgcgatatggtcgacgacgacgagctcatcgagctcgttgaggaagaggtccgtgacctcctcgacgaaaatggcttcgatcgcgattgcccggtcatccacgtgtccgcttacggcgcactgcacgatgacgctccggaccacgagaagtgggttgagcagatcaagaagctcatggacgccgtcgatgactacatcccgaccccggtccacgatctggacaagccgttcctgatgccgatcgaagatgtcttcaccatctccggccgtggcaccgtggtgaccggccgtgtcgagcgtggtaagctcccggtcaactccaacgtcgagatcgtcggcatccgtccgacccagaccaccaccgtcacctccatcgagaccttccacaagcagatggacgagtgcgaggctggcgacaacaccggtctgctgctccgcggcatcaaccgtgaccaggtcgagcgtggccaggttctggctgctccgggctccgtgaccccgcacaccaagttcgagggcgaagtctacgtgctgaccaaggacgaaggcggccgtcactcgccgttcttctccaactaccgtccgcagttctacttccg

SEQ ID NO: 23 - DNA, 776 bp, Bifidobacterium pseudocatenulatum
cacgtcgactgcccgggccacgccgacttcgtgaagaacatgatcactggcgctgcccagatggatggcgctatcctcgttgtggccgccaccgacggcccgatggctcagacccgcgagcacgtgctgctcgctcgtcaggtgggcgtgccgcgtatcctcgtcgctctgaacaagtgcgatatggtcgacgacgaagagctcatcgagcttgttgaggaagaggtccgtgacctcctcgacgaaaacggcttcgatcgcgattgcccggtcatccacacctccgcttacggcgcgctgcacgacgacgctccggatcacgagaagtgggttgagtccgtcaaggaactcatgaaggctgtcgacgagtacatcccgaccccgactcacgatctggacaagccgttcctgatgccgatcgaagatgtcttcaccatctccggccgtggcaccgtggttaccggccgtgtcgagcgtggtaagctcccggtcaactccaacgtcgagatcgttggtatccgtccgacccagaccaccaccgtcacctccatcgagaccttccacaagcagatggatgagtgcgaggctggcgacaacaccggtctgctgctccgcggcatcaaccgtgaccaggtcgagcgtggccaggttctggctgctccgggctccgtgaccccgcacaccaagttcgagggcgaagtctacgtgctgaccaaggacgaaggcggccgtcactcgccgttcttctccaactaccgtccgcagttctacttccg

SEQ ID NO: 24 - DNA, 776 bp, Bifidobacterium breve
cacgtcgactgcccgggccacgccgacttcgtgaagaacatgatcaccggtgctgcccagatggatggcgccatcctcgttgtggccgccaccgacggcccgatggctcagacccgcgagcacgtgctgctcgctcgtcaggtgggcgtcccgaagatccttgtcgccctgaacaagtgcgacatggttgaggatgaggagctcatcgagctcgtcgaagaagaggtccgcgacctcctcgacgagaacggcttcgaccgcgactgcccggtcatccacacctccgcttacggtgctctgcacgacgacgctccggaccacgagaagtgggtccagtccgttaaggacctcatggacgccgtcgacgactacatcccgaccccggtccacgacctcgacaagccgttcctgatgccgatcgaggacgtcttcaccatctccggccgtggtaccgttgtcaccggtcgtgttgagcgtggccagctggccgtcaacactccggtcgagatcgttggtatccgtccgacccagaccaccaccgtcacctccatcgagaccttccacaagaccatggacgcctgcgaggctggcgacaacaccggtctgctgctgcgtggtcttggtcgtgaagatgtcgagcgtggccaggttgtggccaagccgggctccgtcaccccgcacaccaagttcgagggcgaagtctacgtgctgaccaaggacgagggcggccgtcactcgccgttcttctccaactaccgtccgcagttctacttccg

SEQ ID NO: 25 - DNA, 776 bp, Bifidobacterium animalis
cacgtcgactgcccgggccacgccgacttcgtgaagaacatgatcaccggcgctgcccagatggatggcgccatcctcgttgtggccgccaccgacggcccgatggcccagacccgcgagcacgtgctgctcgcccgtcaggtcggcgtcccgaagatcctcgtcgctctgaacaagtgcgatatggtcgatgacgaagagctcatcgagctcgtcgaagaagaggtccgcgacctcctcgacgagaacggcttcgaccgcgactgcccggtcgtgcacacctccgcttacggcgctctgcatgacgacgctccggatcacgacaagtgggttgccaccatcaaggagctcatggacgacgtcgacgagtacatcccgaccccggtccacgacctcgacaagccgttcctgatgccgatcgaggacgtcttcaccatctccggccgtggcaccgtcgtcaccggtcgtgtcgagcgcggcaagctgccgatcaacacgaacgtcgagatcgtcggcatccgcccgacccagaccaccaccgtcacctccatcgagaccttccacaagcagatggatgagtgcgaggccggcgacaacaccggtctgctgctccgcggcatcaaccgcaccgacgtcgagcgtggccaggtcgtggctgctccgggttcggtcaccccgcacaccaagttcgaaggcgaagtctacgtccttaccaaggatgagggcggccgtcactcgccgttcttctcgaactaccgtccgcagttctacttccg

SEQ ID NO: 26 - DNA, 16 bp, Artificial Sequence
  Streptococcus sanguinis DSM20567 - 1SsangL
cactctcacacccgtt

SEQ ID NO: 27 - DNA, 17 bp, Artificial Sequence
  Streptococcus sanguinis DSM20567 - 1SsangL-Q
  misc_feature (17)..(17): conjugation to BHQ2 (Black hole quencher 2)
cactctcacacccgttc

SEQ ID NO: 28 - DNA, 11 bp, Artificial Sequence
  Streptococcus sanguinis DSM20567 - R 3′ - 5′
gagtgtgggca

SEQ ID NO: 29 - DNA, 11 bp, Artificial Sequence
  Streptococcus sanguinis DSM20567 - 1SsangD-610-50
  misc_feature (1)..(1): CAL Fluor Red 610
acgggtgtgag

SEQ ID NO: 30 - DNA, 19 bp, Artificial Sequence
  Clostridium ramosum DSM1402 - 2CramsL
gtcactcggctaccatttc

SEQ ID NO: 31 - DNA, 20 bp, Artificial Sequence
  Clostridium ramosumDSM1402 - 2CramsL-Q
  misc_feature (20)..(20): conjugation to BHQ2 (black hole quencher 2)
gtcactcggctaccatttcc

SEQ ID NO: 32 - DNA, 12 bp, Artificial Sequence
  Clostridium ramosum DSM1402 - R 3′ - 5′
agccgatggtaa

SEQ ID NO: 33 - DNA, 12 bp, Artificial Sequence
  Clostridium ramosum DSM1402 - 2CramsD-560-50
  misc_feature (1)..(1): conjugation to CAL Fluor Orange 560
aatggtagccga

SEQ ID NO: 34 - DNA, 19 bp, Artificial Sequence
  Streptococcus pyogenes  DSM20565 - 3SpyogL
gattttccactcccaccat

SEQ ID NO: 35 - DNA, 20 bp, Artificial Sequence
  Streptococcus pyogenes DSM20565 - 3SpyogL-Q
  misc_feature (20)..(20): conjugation to BHQ2 (black hole quencher 2)
gattttccactcccaccatc

SEQ ID NO: 36 - DNA, 12 bp, Artificial Sequence
  Streptococcus pyogenes DSM20565 - R 3′ - 5′
gtgagggtggta

SEQ ID NO: 37 - DNA, 12 bp, Artificial Sequence
  Streptococcus pyogenes DSM20565 - 3SpyogD-FAM-50
  misc_feature (1)..(1): Conjugation to FAM
atggtgggagtg

SEQ ID NO: 38 - DNA, 15 bp, Artificial Sequence
  Listeria monocytogenes DSM20600 - 4LmonoL
ccgtcaagggacaag

SEQ ID NO: 39 - DNA, 16 bp, Artificial Sequence
  Listeria monocytogenes DSM20600 - 4LmonoL-Q
  misc_feature (16)..(16): conjugation to BHQ2 (black hole quencher 2)
ccgtcaagggacaagc

SEQ ID NO: 40 - DNA, 10 bp, Artificial Sequence
  Listeria monocytogenes DSM20600 - R 3′ - 5′
gcagttccct

SEQ ID NO: 41 - DNA, 10 bp, Artificial Sequence
  Listeria monocytogenes DSM20600 - 4LmonoD-560-40
  misc_feature (1)..(1): conjugation to CAL Fluor Orange 560
tcccttgacg

SEQ ID NO: 42 - DNA, 20 bp, Artificial Sequence
  Bacteroides thetaiotaomicron DSM2079 - 5BthetL
aagacagccagtatcaactg

SEQ ID NO: 43 - DNA, 21 bp, Artificial Sequence
  Bacteroides thetaiotaomicron DSM2079 - 5BthetL-Q
  misc_feature (21)..(21): conjugation to BHQ2 (black hole quencher 2)
aagacagccagtatcaactgc

SEQ ID NO: 44 - DNA, 19 bp, Artificial Sequence
  Bacteroides thetaiotaomicron DSM2079 - R 3′ - 5′
tctgtcggtcatagttgac

SEQ ID NO: 45 - DNA, 19 bp, Artificial Sequence
  Bacteroides thetaiotaomicron DSM2079 - 5BthetD-560-60
  misc_feature (1)..(1): conjugation to CAL Fluor Orange 560
cagttgatactggctgtct

SEQ ID NO: 46 - DNA, 22 bp, Artificial Sequence
  Enterobacter cloacae DSM30054 - 6EcloaL
gcgcccccggggatttcacatc

SEQ ID NO: 47 - DNA, 23 bp, Artificial Sequence
  Enterobacter cloacae DSM30054 - 6EcloaL-Q
  misc_feature (23)..(23): conjugation to BHQ2 (black hole quencher 2)
gcgcccccggggatttcacatcc

SEQ ID NO: 48 - DNA, 20 bp, Artificial Sequence
  Enterobacter cloacae DSM30054 - R 3′ - 5′
gcgggggcccctaaagtgta

SEQ ID NO: 49 - DNA, 20 bp, Artificial Sequence
  Enterobacter cloacae DSM30054 - 6EcloaD-560-70
  misc_feature (1)..(1): conjugation to CAL Fluor Orange 560
atgtgaaatccccgggggcg

SEQ ID NO: 50 - DNA, 30 bp, Artificial Sequence
  Lactobacillus acido DSM20079 - 7LacidL
ggcgcggccccggccacatgctgggttccc

SEQ ID NO: 51 - DNA, 31 bp, Artificial Sequence
  Lactobacillus acido DSM20079 - 7LacidL-Q
  misc_feature (31)..(31): conjugation to BHQ2 (black hole quencher 2)
ggcgcggccccggccacatgctgggttcccc

SEQ ID NO: 52 - DNA, 26 bp, Artificial Sequence
  Lactobacillus acido DSM20079 - R 3′ - 5′
cgcgccggggccggtgtacgacccaa

SEQ ID NO: 53 - DNA, 26 bp, Artificial Sequence
  Lactobacillus acido DSM20079 - 7LacidD-560-80
  misc_feature (1)..(1): conjugation to CAL Fluor Orange 560
aacccagcatgtggccggggccgcgc

SEQ ID NO: 54 - DNA, 18 bp, Artificial Sequence
  16S rRNA PCR primers - 16SV3V9F
tcctacgggaggcagcag

SEQ ID NO: 55 - DNA, 20 bp, Artificial Sequence
  16S rRNA PCR primers - 16SV3V9R
cggttaccttgttacgactt

SEQ ID NO: 56 - DNA, 13 bp, Artificial Sequence
  Bifidobacterium longum DSM20219 - 8BlongL
cgcgctcgacatc

SEQ ID NO: 57 - DNA, 14 bp, Artificial Sequence
  Bifidobacterium longum DSM20219 - 8BlongL-Q
  misc_feature (14)..(14): conjugation to BHQ2 (black hole quencher 2)
cgcgctcgacatcc

SEQ ID NO: 58 - DNA, 21 bp, Artificial Sequence
  Bifidobacterium breve DSM20213 - 9Bbrev1L
ggaatcgatgagctcctcatc

SEQ ID NO: 59 - DNA, 22 bp, Artificial Sequence
  Bifidobacterium breve DSM20213 - 9Bbrev1L-Q
  misc_feature (22)..(22): conjugation to BHQ2 (black hole quencher 2)
ggaatcgatgagctcctcatcc

SEQ ID NO: 60 - DNA, 20 bp, Artificial Sequence
  Bifidobacterium breve DSM20213 - R 3′ - 5′
ccttagctactcgaggagta

SEQ ID NO: 61 - DNA, 20 bp, Artificial Sequence
  Bifidobacterium breve DSM20213 - 9Bbrev1D-FAM-60
  misc_feature (1)..(1): conjugation to FAM
atgaggagctcatcgattcc

SEQ ID NO: 62 - DNA, 21 bp, Artificial Sequence
  Bifidobacterium breve DSM20213 - 9Bbrev2L
gcttgacatggttgaggatga

SEQ ID NO: 63 - DNA, 22 bp, Artificial Sequence
  Bifidobacterium breve DSM20213 - 9Bbrev2L-Q
  misc_feature (22)..(22): conjugation to BHQ2 (black hole quencher 2)
gcttgacatggttgaggatgac

SEQ ID NO: 64 - DNA, 20 bp, Artificial Sequence
  Bifidobacterium breve DSM20213 - R 3′ - 5′
cgaactgtaccaactcctac

SEQ ID NO: 65 - DNA, 20 bp, Artificial Sequence
  Bifidobacterium breve DSM20213 - 9Bbrev2D-FAM-60
  misc_feature (1)..(1): conjugation to FAM
catcctcaaccatgtcaagc

SEQ ID NO: 66 - DNA, 14 bp, Artificial Sequence
  Bifido animalis DSM10140 - 10BanimL
gacaagtgggttgc

SEQ ID NO: 67 - DNA, 15 bp, Artificial Sequence
  Bifido animalis DSM10140 - 10BanimL-Q
  misc_feature (15)..(15): conjugation to BHQ2 (black hole quencher 2)
gacaagtgggttgcc

SEQ ID NO: 68 - DNA, 10 bp, Artificial Sequence
  Bifido animalis DSM10140 - R 3′ - 5′
gttcacccaa

SEQ ID NO: 69 - DNA, 10 bp, Artificial Sequence
  Bifido animalis DSM10140 - 10BanimD-610-40
  misc_feature (1)..(1): conjugation to CAL Fluor Red 610
aacccacttg

SEQ ID NO: 70 - DNA, 17 bp, Artificial Sequence
  Bifido adolescentis DSM20083 - 11BadolL
ggaaccgtaagcggaca

SEQ ID NO: 71 - DNA, 18 bp, Artificial Sequence
  Bifido adolescentis DSM20083 - 11BadolL-Q
  misc_feature (18)..(18): conjugation to BHQ2 (black hole quencher 2)
ccttccgtaagcggacac

SEQ ID NO: 72 - DNA, 16 bp, Artificial Sequence
  Bifido adolescentis DSM20083 - R 3′ - 5′
ggaaggcattcgcctg

SEQ ID NO: 73 - DNA, 16 bp, Artificial Sequence
  Bifido adolescentis DSM20083 - 11BadolD-610-60
  misc_feature (1)..(1): CAL Fluor Red 610
gtccgcttacggaagg

SEQ ID NO: 74 - DNA, 15 bp, Artificial Sequence
  Bifido pseudocatenulat DSM20438 - 12BpseuL
cgcgcccgaccccga

SEQ ID NO: 75 - DNA, 16 bp, Artificial Sequence
  Bifido pseudocatenulat DSM20438 - 12BpseuL-Q
  misc_feature (16)..(16): conjugation to BHQ2 (black hole quencher 2)
cgcgcccgaccccgac

SEQ ID NO: 76 - DNA, 15 bp, Artificial Sequence
  Bifido pseudocatenulat DSM20438 - R 3′ - 5′
gcgcgggctggggct

SEQ ID NO: 77 - DNA, 15 bp, Artificial Sequence
  Bifido pseudocatenulat DSM20438 - 12BpseuD-610-70
  misc_feature (1)..(1): conjugation to CAL Fluor Red 610
tcggggtcgggcgcg

SEQ ID NO: 78 - DNA, 12 bp, Artificial Sequence
  Amplicon 4 - PCR primers 1/3-17 - Bifi4F
gcccgggccacg

SEQ ID NO: 79 - DNA, 15 bp, Artificial Sequence
  Amplicon 4 - PCR primers 1/3-17 - BIFI4R
tagaactgcggacgg

(19) United States
(12) Patent Application Publication
(10) Pub. No.: US 2022/0041685 A1
(43) Pub. Date: Feb. 10, 2022

(54) ENGINEERED T-CELL MODULATING MOLECULES AND METHODS OF USING SAME

(71) Applicant: SUNNYBROOK RESEARCH INSTITUTE, Toronto (CA)

(72) Inventors: Aaron PRODEUS, Stoney Creek (CA); Jean GARIEPY, Toronto (CA); Mays Abdulkaree ALWASH, Mississauga (CA)

(21) Appl. No.: 17/510,761

(22) Filed: Oct. 26, 2021

Related U.S. Application Data
(63) Continuation of application No. 16/498,586, filed on Sep. 27, 2019.
(60) Provisional application No. 62/478,198, filed on Mar. 29, 2017.
(60) Provisional application No. 62/590,848, filed on Nov. 27, 2017.

(51) Int. Cl. C07K 14/705; C07K 14/47; C12N 5/0783

(52) U.S. Cl. CPC C07K 14/705; C07K 14/47; C12N 2501/998; C07K 2319/03; C12N 5/0636

(57) ABSTRACT

Provided herein are engineered nucleic acids, engineered mRNAs, engineered polypeptides, and engineered pentamerized polypeptides, human and murine, each of which include sequences of VISTA, ICOS-L, PD-L1 or B7-H4 extracellular domains operably linked to the pentamerization domain of COMP. A soluble form of the pentamerized polypeptides has T-cell inhibitory activity, in the case of VISTA, PD-L1 or B7H4, or T-cell stimulatory activity, in the case of ICOS-L. Methods of using same for treatment of a subject in need of T-cell modulating activity are provided.

### CROSS REFERENCE TO PRIOR APPLICATIONS

This application claims priority under the Paris Convention to U.S. Provisional Patent Application 62/478,198, filed Mar. 29, 2017, and U.S. Provisional Patent Application 62/590,848, filed Nov. 27, 2017, each of which are incorporated herein by reference as if set forth in their entirety.

### FIELD OF THE DISCLOSURE

This present disclosure relates generally to engineered T-cell modulating molecules. In particular, the present disclosure is directed to engineered V-domain Immunoglobulin suppressor of T-cell activation (VISTA), programmed death-ligand 1 (PD-L1) and B7 homolog 4 (B7-H4) molecules that inhibit T-cells and engineered inducible costimulatory ligand (ICOS-L) molecules that stimulate T-cells.

### BACKGROUND OF THE DISCLOSURE

T-cell activity is regulated by co-stimulatory and co-inhibitory signals generated by the binding of immune checkpoint (IC) cell surface molecules present on T-cells and antigen presenting cells (APC)/cancer cells. These signals facilitate protection against invading pathogens and/or malignant cells, while maintaining self-tolerance. T-cell responses are either upregulated by co-stimulatory checkpoint pairs exemplified by CD28:CD80/CD86, ICOS:ICOS-L, OX-40:OX-40L and 4-1BB:4-1BBL and/or downregulated by co-inhibitory checkpoint molecules which include CTLA-4:CD80/CD86, PD-1:PD-L1 (References 1-4). Some T cell co-inhibitory IC pairs have not yet been fully characterized including the receptors recognized by VISTA and B7-H4. To date, several negative checkpoint receptors that function to suppress T-cell activity have been identified, including PD-1 and CTLA-4. Antibody-mediated blockade of these pathways has been shown to promote anti-tumor immune responses (References 1-3) while ligands which activate these immunoinhibitory pathways may suppress uncontrolled immune responses linked to autoimmune and/or inflammatory disorders (References 4-6). In contrast, activation of co-stimulatory pathways in T-cells such as ICOS:ICOS-L and OX40:OX40L can lead to T-cell activation, proliferation and cytokine production; outcomes which can promote anti-tumor immune responses.

Many of the known immune checkpoint pairs are members of the B7-CD28 family of surface proteins defined in part by their Ig-V/Ig-C containing extracellular domains. These pairs include ICOS:ICOS-L, CTLA-4:CD80/CD86, PD-1:PD-L1, as well as VISTA and B7-H4 (Reference 7).

V-domain Immunoglobulin suppressor of T-cell activation (“VISTA”, which may also be referred to as PD-1H, DD1α, SISP1, Dies1, c10Orf54, and/or Gi24) is a checkpoint ligand that is expressed primarily on CD11bhigh myeloid cells and which negatively regulates T-cell responses upon binding to a putative cell surface receptor (VISTA-receptor) (References 8-9). VISTA is also expressed on naïve CD4+ and CD8+ T-cells, where it is postulated to negatively regulate T-cell responses, suggesting a dual-role of VISTA as both a checkpoint ligand and receptor (Reference 10). Structurally, VISTA shares significant homology with PD-1 and PD-L1, having an N-terminal IgV domain followed by a single membrane spanning domain and cytoplasmic tail. Similar to the PD-1:PD-L1 pathway, blockade of VISTA using monoclonal antibodies has been demonstrated to provoke anti-tumor immune responses in mouse models, suggesting a role for VISTA:VISTA-receptor signalling in the promotion of tumor immune evasion (References 11-12). VISTA may also play a role in regulating autoimmune disease progression. For example, VISTA-deficient (VISTA−/−) mice bred on a lupus-prone background developed accelerated and severe systemic lupus erythematosus (SLE) (Reference 13). VISTA−/− 2D2 T-cell receptor transgenic mice exhibited increased levels of peripheral encephalitogenic T-cells and developed an exacerbated form of experimental autoimmune encephalomyelitis (EAE) (Reference 14). VISTA−/− mice bred on a C57Bl/6 background displayed a mild pro-inflammatory phenotype, exemplified by an increase in dendritic cells and a rise in T-cell activation markers, but were not reported to develop inflammatory disorders (Reference 14). In slight contrast, another study described a more severe phenotype, where VISTA−/− C57Bl/6 mice developed glomerulonephritis at 10 months of age (Reference 15). Together, these studies suggest that promoting VISTA-mediated immunosuppression may be useful for treatment of autoimmune and/or inflammatory diseases.

Agonistic anti-VISTA antibodies have been reported (Reference 16) and a dimeric version of VISTA (VISTA. Fc) has been reported to suppress T-cell activation in-vitro when VISTA.Fc is immobilized on a solid surface (References 8, 11).

The inhibitory co-stimulatory molecule known as programmed death-1 (PD-1) is expressed on activated T cells, B cells, monocytes, and macrophages and binds to PD-L1 (on hematopoietic and non-hematopoietic cells) and PD-L2 (on DCs and macrophages) (References 6, 17-20). PD-L1 binding to PD-1 on lymphocytes sends an inhibitory signal to T cells that blocks TCR signaling, T- and B-cell proliferation, cytokine production and CD8+ T cell cytotoxicity (References 4, 17). PDL-2 is a second ligand for PD-1 and inhibits T-cell activation (Reference 20). The PD-1 agonist PD-L1.Fc has been shown to improve disease outcome in two CIA mouse models (References 5, 21).

B7-H4 is another B7 family member that is an IgV domain-containing inhibitory ligand. Its receptor first presumed to be BTLA-4 still remains unknown (Reference 22). B7-H4.Fc has been shown to dampen immune responses in vivo, exemplified by its ability to reduce ConA-induced hepatic injury in mice (Reference 23) and to limit the progression of CIA in mice (Reference 24).

The Inducible T-cell Costimulator ((ICOS), which may also be referred to as CD278, H4 or AILIM) is a receptor in the CD28 family of B7-binding proteins (References 25-27) which is inducibly expressed on activated T cells (References 25, 28, 29). Upon binding to its ligand ICOS-L (B7-H2) expressed on APCs (References 30, 31), T-cells are co-stimulated by ICOS to enhance Th1 and Th2 functions reflected by the production of effector cytokines (IL-4, IL-5, IL-10, IL-21, IFNγ, TNFα) (References 32-34).

Pre-clinical tumor studies have shown that mice implanted with tumor cells expressing ICOS-L (to agonize ICOS signalling) have reduced tumor growth and improved survival in the context of anti-CTLA-4 therapy (Reference 35). As well, a clinical trial of patients treated with anti-CTLA-4 agents have shown that the presence of ICOS-hi T cells correlates with an increased treatment response to these immune checkpoint inhibitors (References 36, 37). These results suggest the use of an ICOS agonist as a strategy to enhance anti-tumor immune response. PCT Application No. WO2016US23524 describes agonistic monoclonal antibodies that target ICOS. These antibodies are purported to both stimulate immune cells to kill tumor cells, and to limit the number of Treg linked to the suppression of antitumor immunity.

There is currently a need for improved compounds and methods for regulating or modulating T-cell activity, inducing immunosuppression and/or improving anti-tumor immune responses.

### SUMMARY OF THE DISCLOSURE

Provided herein are engineered nucleic acids, engineered mRNAs, engineered polypeptides, and engineered pentamerized polypeptides, human and murine, each of which includes a sequence of a VISTA, B7-H4, PD-L1 or ICOS-L extracellular domain operably linked to the pentamerization domain of COMP. A soluble form of the pentamerized polypeptides has T-cell modulating activity in vitro and in vivo. Methods of using same for treatment of a subject in need of T-cell modulating activity are also provided.

In an aspect, a recombinant nucleic acid is provided. The recombinant nucleic acid comprises: a nucleic acid having substantial similarity to a nucleic acid encoding an extracellular IgV-containing domain of a V-domain Ig Suppressor of T cell Activation (VISTA) having a sequence of SEQ ID NO: 1 or 2; and a nucleic acid having substantial similarity to a nucleic acid encoding a pentamerization domain of cartilage oligomeric matrix protein (COMP) having a sequence of SEQ ID NO: 3 or 4, the nucleic acid encoding the extracellular IgV-domain containing VISTA polypeptide being operably linked to the nucleic acid encoding the pentamerization domain of COMP.

In an embodiment, the recombinant nucleic acid comprises SEQ ID NO: 1 operably linked to SEQ ID NO: 3. In an embodiment, the recombinant nucleic acid comprises SEQ ID NO: 2 operably linked to SEQ ID NO: 4.

In another aspect, a recombinant nucleic acid is provided. The recombinant nucleic acid comprises: a nucleic acid having substantial similarity to a nucleic acid encoding an extracellular domain of B7-H4 having a sequence of SEQ ID NO: 26; and a nucleic acid having substantial similarity to a nucleic acid encoding a pentamerization domain of cartilage oligomeric matrix protein (COMP) having a sequence of SEQ ID NO: 3, the nucleic acid encoding the extracellular domain of B7-H4 polypeptide being operably linked to the nucleic acid encoding the pentamerization domain of COMP.

In another aspect, a recombinant nucleic acid is provided. The recombinant nucleic acid comprises: a nucleic acid having substantial similarity to a nucleic acid encoding an extracellular domain of PD-L1 having a sequence of SEQ ID NO: 37; and a nucleic acid having substantial similarity to a nucleic acid encoding a pentamerization domain of cartilage oligomeric matrix protein (COMP) having a sequence of SEQ ID NO: 3, the nucleic acid encoding the extracellular domain of PD-L1 polypeptide being operably linked to the nucleic acid encoding the pentamerization domain of COMP.

In another aspect, a recombinant nucleic acid is provided. The recombinant nucleic acid comprises: a nucleic acid having substantial similarity to a nucleic acid encoding an extracellular domain of ICOS-L having a sequence of SEQ ID NO: 48; and a nucleic acid having substantial similarity to a nucleic acid encoding a pentamerization domain of cartilage oligomeric matrix protein (COMP) having a sequence of SEQ ID NO: 3, the nucleic acid encoding the extracellular domain of ICOS-L polypeptide being operably linked to the nucleic acid encoding the pentamerization domain of COMP.

In an embodiment, an expression vector comprising the recombinant nucleic acid of the disclosure is provided. In an embodiment, the expression vector further comprises at least one control sequence. In an embodiment, a host cell comprising the expression vector is provided.

In an aspect, a recombinant messenger ribonucleic acid (mRNA) is provided. The mRNA comprises: an mRNA having substantial similarity to an mRNA encoding an extracellular domain of a V-domain Ig Suppressor of T cell Activation (VISTA) having a sequence of SEQ ID NO: 5 or 6; and an mRNA having substantial similarity to an mRNA encoding a pentamerization domain of cartilage oligomeric matrix protein (COMP) having a sequence of SEQ ID NO: 7 or 8, the mRNA encoding the extracellular domain of VISTA mRNA being operably linked to the mRNA encoding the pentamerization domain of COMP.

In an embodiment, the recombinant mRNA comprises SEQ ID NO: 5 operably linked to SEQ ID NO: 7. In an embodiment, the recombinant mRNA comprises SEQ ID NO: 6 operably linked to SEQ ID NO: 8.

In an aspect, a recombinant messenger ribonucleic acid (mRNA) is provided. The mRNA comprises: an mRNA having substantial similarity to an mRNA encoding an extracellular domain of B7-H4 having a sequence of SEQ ID NO: 27; and an mRNA having substantial similarity to an mRNA encoding a pentamerization domain of cartilage oligomeric matrix protein (COMP) having a sequence of SEQ ID NO: 7, the mRNA encoding the extracellular domain of B7-H4 mRNA being operably linked to the mRNA encoding the pentamerization domain of COMP.

In an aspect, a recombinant messenger ribonucleic acid (mRNA) is provided. The mRNA comprises: an mRNA having substantial similarity to an mRNA encoding an extracellular domain of PD-L1 having a sequence of SEQ ID NO: 62; and an mRNA having substantial similarity to an mRNA encoding a pentamerization domain of cartilage oligomeric matrix protein (COMP) having a sequence of SEQ ID NO: 7, the mRNA encoding the extracellular domain of PD-L1 mRNA being operably linked to the mRNA encoding the pentamerization domain of COMP.

In an aspect, a recombinant messenger ribonucleic acid (mRNA) is provided. The mRNA comprises: an mRNA having substantial similarity to an mRNA encoding an extracellular domain of ICOS-L having a sequence of SEQ ID NO: 61; and an mRNA having substantial similarity to an mRNA encoding a pentamerization domain of cartilage oligomeric matrix protein (COMP) having a sequence of SEQ ID NO: 7, the mRNA encoding the extracellular domain of ICOS-L mRNA being operably linked to the mRNA encoding the pentamerization domain of COMP.

In an aspect, a recombinant polypeptide is provided. The recombinant polypeptide comprises: a polypeptide having substantial similarity to an extracellular domain of a V-domain Ig Suppressor of T cell Activation (VISTA) (SEQ ID NO: 9 or 10) linked to a polypeptide having substantial similarity to a pentamerization domain of cartilage oligomeric matrix protein (COMP) (SEQ ID NO: 11 or 12).

In an embodiment, the recombinant polypeptide comprises SEQ ID NO: 9 operably linked to SEQ ID NO: 11. In an embodiment, the recombinant polypeptide comprises SEQ ID NO: 10 operably linked to SEQ ID NO: 12.

In another aspect, a recombinant polypeptide is provided. The recombinant polypeptide comprises: a polypeptide having substantial similarity to an extracellular domain of B7-H4 (SEQ ID NO: 25) linked to a polypeptide having substantial similarity to a pentamerization domain of cartilage oligomeric matrix protein (COMP) (SEQ ID NO: 11).

In another aspect, a recombinant polypeptide is provided. The recombinant polypeptide comprises: a polypeptide having substantial similarity to an extracellular domain of PD-L1 (SEQ ID NO: 36) linked to a polypeptide having substantial similarity to a pentamerization domain of cartilage oligomeric matrix protein (COMP) (SEQ ID NO: 11).

In another aspect, a recombinant polypeptide is provided. The recombinant polypeptide comprises: a polypeptide having substantial similarity to an extracellular domain of ICOS-L (SEQ ID NO: 49) linked to a polypeptide having substantial similarity to a pentamerization domain of cartilage oligomeric matrix protein (COMP) (SEQ ID NO: 11).

In an embodiment, a recombinant polypeptide provided herein is in a soluble form.

In an aspect, a pentamerized polypeptide having T-cell inhibitory activity is provided. The pentamerized polypeptide having T-cell inhibitory activity comprises: five monomers, each of the monomers comprising: a polypeptide having substantial similarity to an extracellular domain of a V-domain Ig Suppressor of T cell Activation (VISTA) (SEQ ID NO: 9 or 10) linked to a polypeptide having substantial similarity to a pentamerization domain of cartilage oligomeric matrix protein (COMP) (SEQ ID NO: 11 or 12).

In an embodiment, the recombinant polypeptide comprises SEQ ID NO: 9 operably linked to SEQ ID NO: 11. In an embodiment, the pentamerized polypeptide comprises SEQ ID NO: 10 operably linked to SEQ ID NO: 12. In an embodiment, the pentamerized polypeptide is in a soluble form. In an embodiment, the soluble form pentamerized polypeptide has increased T-cell inhibitory activity relative to a soluble dimerized polypeptide comprising an extracellular domain of VISTA (SEQ ID NO: 9 or 10). In an embodiment, the increased T-cell inhibitory activity comprises one or more of increased inhibition of T-cell activation and T-cell proliferation. In an embodiment, the soluble form pentamerized polypeptide has increased immune inhibitory activity in vivo relative to a soluble dimerized polypeptide comprising an extracellular domain of VISTA (SEQ ID NO: 9 or 10). In an embodiment, the increased immune inhibitory activity comprises one or more of increased inhibition of cytokine secretion and cytotoxic lymphocyte (CTL) production.

In another aspect, a pentamerized polypeptide having T-cell inhibitory activity is provided. The pentamerized polypeptide having T-cell inhibitory activity comprises: five monomers, each of the monomers comprising: a polypeptide having substantial similarity to an extracellular domain of B7-H4 (SEQ ID NO: 25) linked to a polypeptide having substantial similarity to a pentamerization domain of cartilage oligomeric matrix protein (COMP) (SEQ ID NO: 11).

In an embodiment, the pentamerized polypeptide is in a soluble form. In an embodiment, the soluble form pentamerized polypeptide has increased T-cell inhibitory activity relative to a soluble dimerized polypeptide comprising an extracellular domain of B7-H4 (SEQ ID NO: 25). In an embodiment, the increased T-cell inhibitory activity comprises one or more of increased inhibition of T-cell activation and T-cell proliferation. In an embodiment, the soluble form pentamerized polypeptide has increased immune inhibitory activity in vivo relative to a soluble dimerized polypeptide comprising an extracellular domain of B7-H4 (SEQ ID NO: 25). In an embodiment, the increased immune inhibitory activity comprises one or more of increased inhibition of cytokine secretion and cytotoxic lymphocyte (CTL) production.

In another aspect, a pentamerized polypeptide having T-cell inhibitory activity is provided. The pentamerized polypeptide having T-cell inhibitory activity comprises: five monomers, each of the monomers comprising: a polypeptide having substantial similarity to an extracellular domain of PD-L1 (SEQ ID NO: 36) linked to a polypeptide having substantial similarity to a pentamerization domain of cartilage oligomeric matrix protein (COMP) (SEQ ID NO: 11).

In an embodiment, the pentamerized polypeptide is in a soluble form. In an embodiment, the soluble form pentamerized polypeptide has increased T-cell inhibitory activity relative to a soluble dimerized polypeptide comprising an extracellular domain of PD-L1 (SEQ ID NO: 36). In an embodiment, the increased T-cell inhibitory activity comprises one or more of increased inhibition of T-cell activation and T-cell proliferation. In an embodiment, the soluble form pentamerized polypeptide has increased immune inhibitory activity in vivo relative to a soluble dimerized polypeptide comprising an extracellular domain of PD-L1 (SEQ ID NO: 36). In an embodiment, the increased immune inhibitory activity comprises one or more of increased inhibition of cytokine secretion and cytotoxic lymphocyte (CTL) production.

In another aspect, a pentamerized polypeptide having T-cell stimulatory activity is provided. The pentamerized polypeptide having T-cell stimulatory activity comprises: five monomers, each of the monomers comprising: a polypeptide having substantial similarity to an extracellular domain of ICOS-L (SEQ ID NO: 49) linked to a polypeptide having substantial similarity to a pentamerization domain of cartilage oligomeric matrix protein (COMP) (SEQ ID NO: 11).

In an embodiment, the pentamerized polypeptide is in a soluble form. In an embodiment, the soluble form pentamerized polypeptide has increased T-cell stimulatory activity relative to a soluble dimerized polypeptide comprising an extracellular domain of ICOS-L (SEQ ID NO: 49). In an embodiment, the increased T-cell stimulatory activity comprises one or more of increased stimulation of T-cell activation and T-cell proliferation. In an embodiment, the soluble form pentamerized polypeptide has increased immune stimulatory activity in vivo relative to a soluble dimerized polypeptide comprising an extracellular domain of ICOS-L (SEQ ID NO: 49). In an embodiment, the increased immune stimulatory activity comprises one or more of increased stimulation of cytokine secretion and cytotoxic lymphocyte (CTL) production. In an embodiment, the soluble form pentamerized polypeptide increases effector T-cells:regulatory T-cell ratios.

In an aspect, a pharmaceutical composition is provided. The pharmaceutical composition comprises: one or more of the polypeptides provided herein, the host cells provided herein, and the pentamerized polypeptides provided herein; and a pharmaceutically acceptable carrier, diluent, or excipient.

In an aspect, a method of eliciting a biological response in an individual in need thereof is provided. The method comprises: administering to the individual a therapeutically effective amount of a V-domain Ig Suppressor of T cell Activation (VISTA)-cartilage oligomeric matrix protein (COMP) fusion polypeptide (VISTA.COMP), the VISTA.COMP polypeptide having SEQ ID NO: 9 and being linked to SEQ ID NO: 11 or having SEQ ID NO: 10 and being linked to SEQ ID NO: 12, wherein the biological response is one or more of: suppression of T-cell activation; suppression of T-cell proliferation; decreased secretion by T-cells of one or more inflammatory cytokines; suppressed induction of cytotoxic T lymphocytes (CTLs); and an increase in T-cells with regulatory phenotypes. In an embodiment, the inflammatory cytokines comprise one or more of IL-2 and IFNγ.

In an aspect a method of eliciting a biological response in an individual in need thereof is provided. The method comprises administering to the individual a therapeutically effective amount of a B7-H4-cartilage oligomeric matrix protein (COMP) fusion polypeptide (B7-H4.COMP), the B7-H4.COMP polypeptide having SEQ ID NO: 25 and being linked to SEQ ID NO: 11, wherein the biological response is one or more of: suppression of T-cell activation; suppression of T-cell proliferation; decreased secretion by T-cells of one or more inflammatory cytokines; suppressed induction of cytotoxic T lymphocytes (CTLs); and an increase in T-cells with regulatory phenotypes.

In an aspect, a method of eliciting a biological response in an individual in need thereof is provided. The method comprises administering to the individual a therapeutically effective amount of a PD-L1-cartilage oligomeric matrix protein (COMP) fusion polypeptide (PD-L1.COMP), the PD-L1.COMP polypeptide having SEQ ID NO: 36 and being linked to SEQ ID NO: 11, wherein the biological response is one or more of suppression of T-cell activation; suppression of T-cell proliferation; decreased secretion by T-cells of one or more inflammatory cytokines; suppressed induction of cytotoxic T lymphocytes (CTLs); and an increase in T-cells with regulatory phenotypes.

In an aspect, a method of eliciting a biological response in an individual in need thereof is provided. The method comprises administering to the individual a therapeutically effective amount of an ICOS-L-cartilage oligomeric matrix protein (COMP) fusion polypeptide (ICOS-L.COMP), the ICOS-L.COMP polypeptide having SEQ ID NO: 49 and being linked to SEQ ID NO: 11, wherein the biological response is one or more of stimulation of T-cell activation; stimulation of T-cell proliferation; increased secretion by T-cells of one or more inflammatory cytokines; increased induction of cytotoxic T lymphocytes (CTLs); and an increase in the effector T-cells:regulatory T-cell ratio within the tumor microenvironment. In an embodiment, the ICOS-L.COMP polypeptide is administered in combination with a checkpoint blocking molecule. In an embodiment, the ICOS-L.COMP polypeptide is administered simultaneously with or before or after the checkpoint blocking molecule. In an embodiment, the checkpoint blocking molecule is an anti-PD-1 antibody or an anti-CTLA-4 antibody.

### BRIEF DESCRIPTION OF THE DRAWINGS

The features of the invention will become more apparent in the following detailed description in which reference is made to the appended drawings wherein:

FIGS. 1A-G illustrate that pentameric VISTA.COMP, but not a dimeric VISTA-Fc, suppresses T-cell activation and proliferation as a soluble ligand in-vitro.

FIG. 1A depicts CFSE-labelled purified murine CD4+ T-cells that were activated by plate-bound anti-CD3 antibody (2.5 μg/mL) in the presence (dark grey) or absence (light grey) of immobilized (left panel) or soluble (right panel) VISTA.Fc (10 μg/mL) for 48 hrs. VISTA.Fc suppressed the proliferation of CD4+ T-cells when immobilized, but not when added as a soluble ligand in the culture media.

FIG. 1B shows that recombinant VISTA.COMP was expressed as described in the Methods, and that the purity and pentameric status was confirmed by SDS-PAGE and Western blot in the presence or absence of a reducing agent (DTT). Reduced VISTA.COMP migrated as a single band ˜50 kDa and the disulphide-stabilized pentamer had an apparent mass of 250 kDa.

FIG. 1C depicts results of a proliferation assay of CD4+ T-cells having undergone activation in the presence of coated (9 μg/mL, left panels) or soluble (12 μg/mL, right panels) VISTA.COMP (dark grey) or COMP (light grey). Soluble VISTA.COMP suppressed T-cell expansion (top, FSC & SSC scatter profiles) and proliferation (bottom, CFSE dilution).

FIGS. 1D and 1E depict analysis of culture medium harvested from CD4+ T-cells 48 and 72 hours post anti-CD3 activation in the presence of COMP or VISTA.COMP (10 μg/mL), in which IL-2 (FIG. 1D) and IFNγ (FIG. 1E) secretion were quantified by ELISA. In addition to proliferation, VISTA.COMP was found to significantly suppress IL-2 and IFNγ secretion from T-cells in-vitro (***p<0.005 relative to COMP control, n=3).

FIG. 1F shows that CFSE-labelled CD4+ T-cells were activated with immobilized anti-CD3 antibody at the indicated concentration in the presence of COMP (light grey) or VISTA.COMP (dark grey). VISTA.COMP suppression of T-cell proliferation was overcome by stronger levels of TCR stimulation.

FIG. 1G depicts results of allogenic MLC assays, in which addition of VISTA.COMP significantly suppressed CTL induction in responder cells from wild-type or CD200R1−/− mice (n=3, *p<0.05 relative to CD200Fc).

FIG. 1H shows SDS-PAGE of the expressed pentameric form of human VISTA (hVISTA-COMP) and mVISTA-COMP.

FIG. 11 shows the expansion (top) and CFSE proliferation (bottom) of human T-cells stimulated in-vitro with Concanclavin A in the presence of soluble COMP or hVISTA.COMP. VISTA-COMP suppresses the proliferation induced in these T-cells.

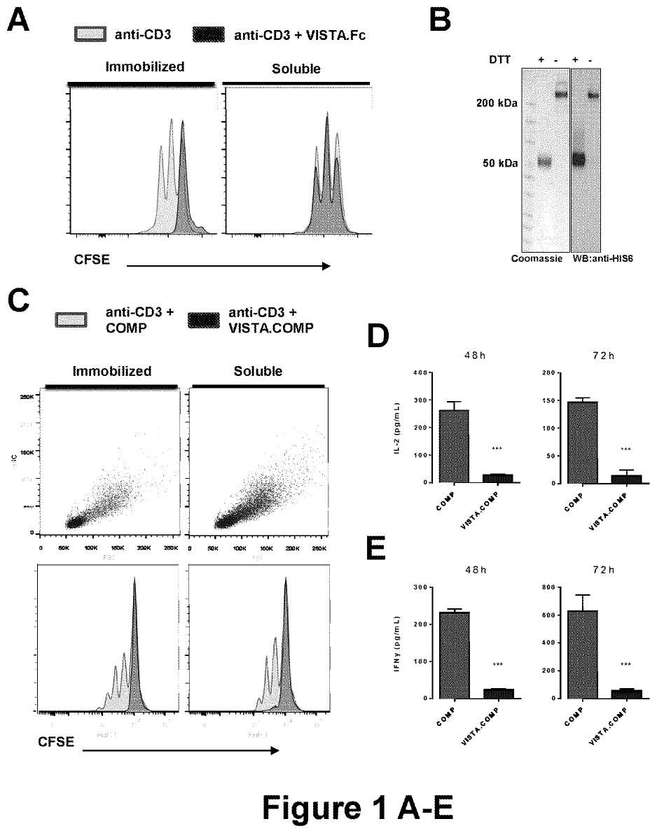

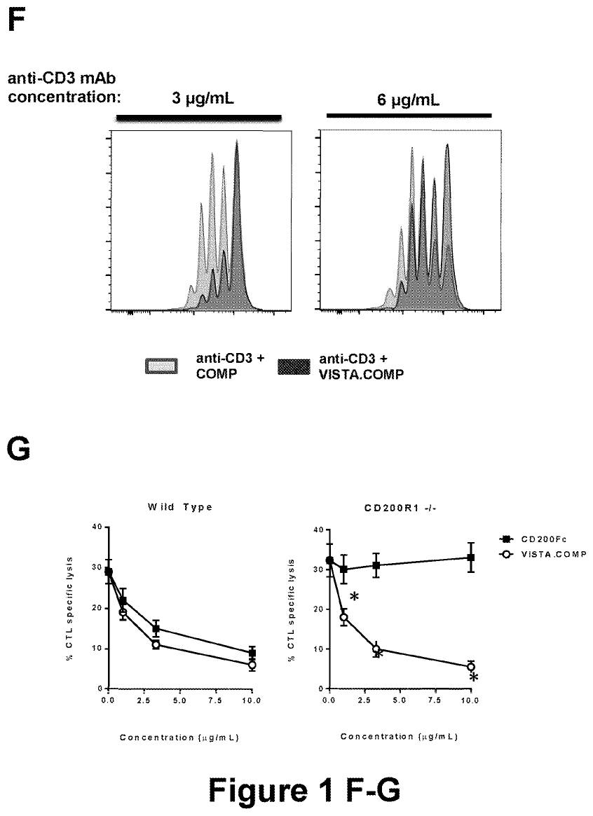

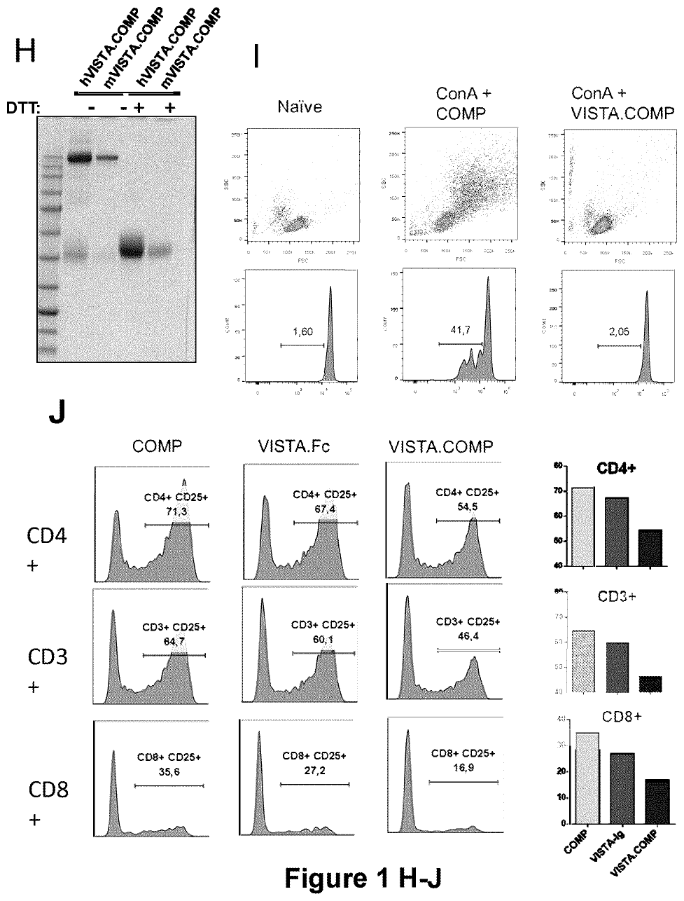

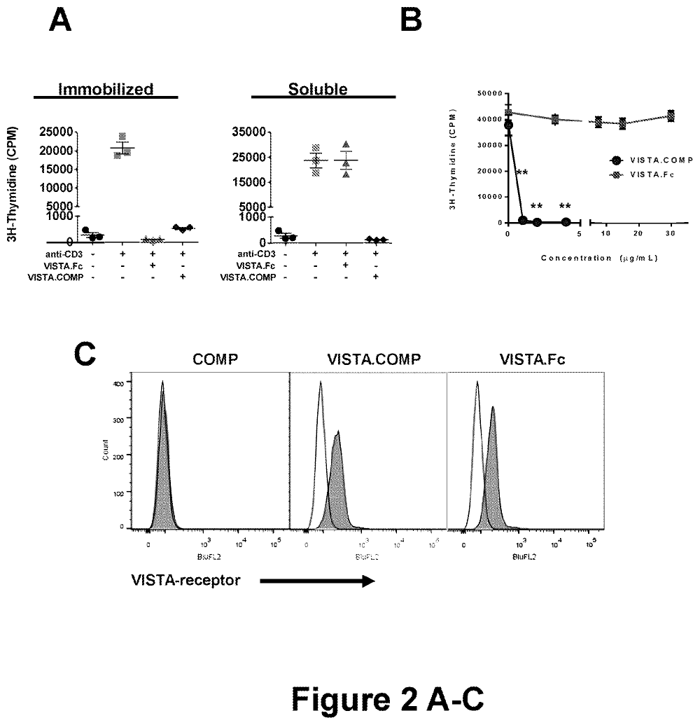

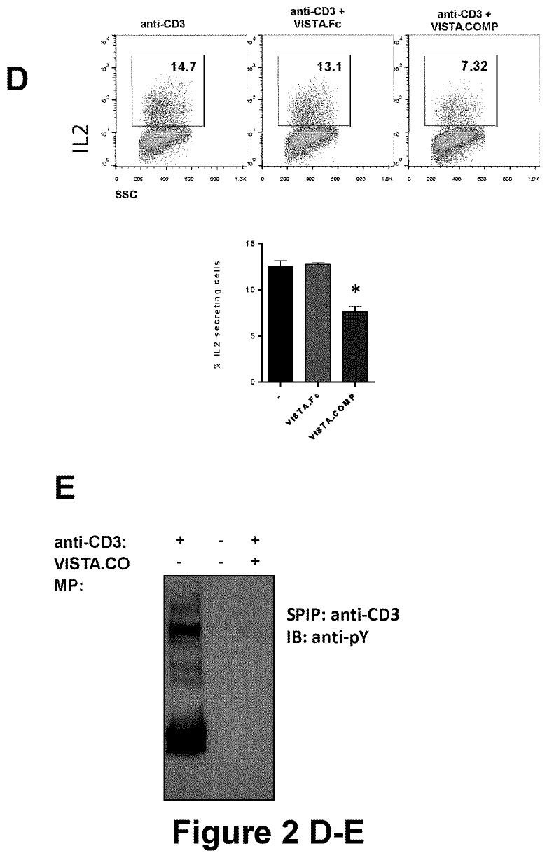

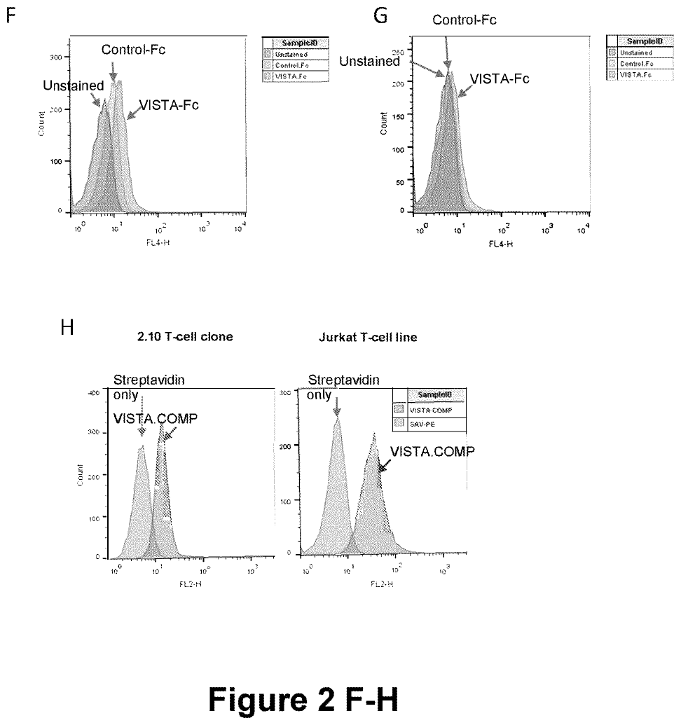

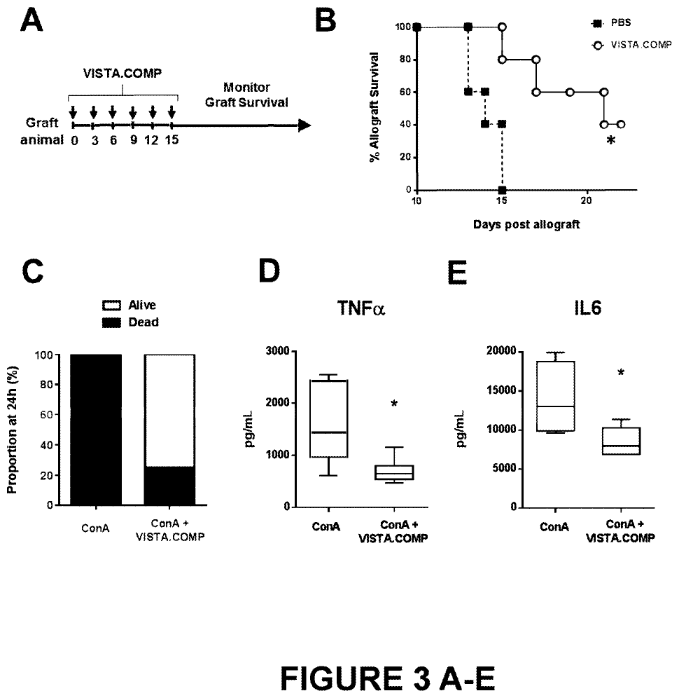

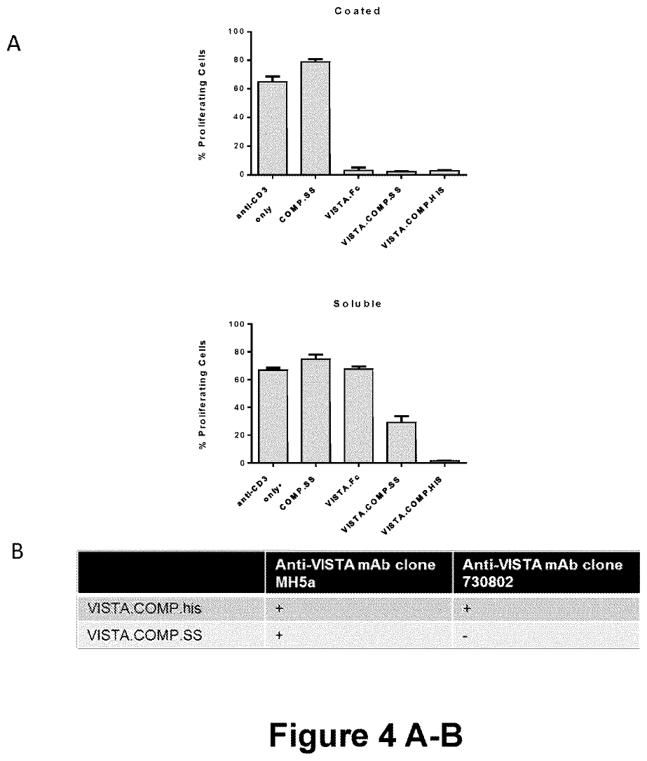

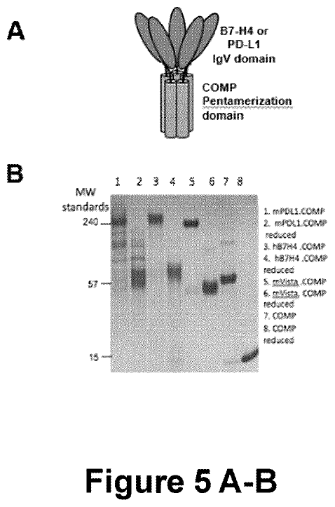

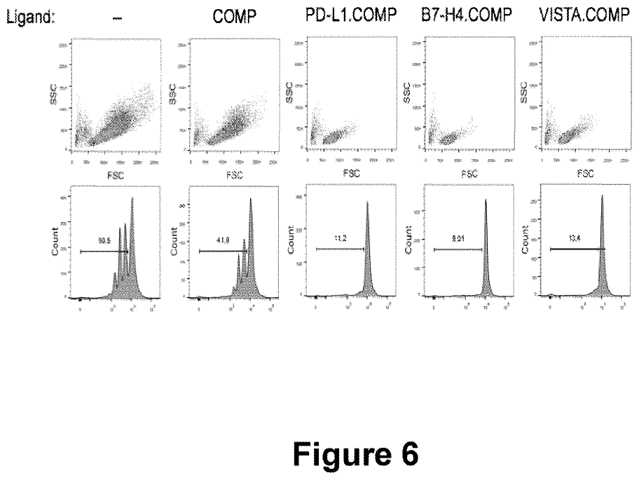

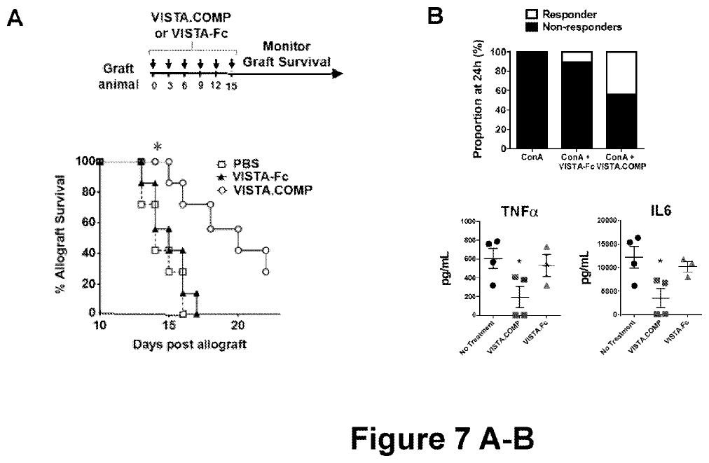

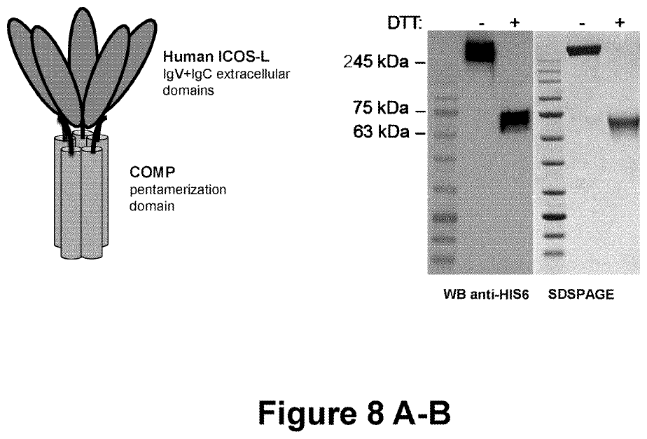

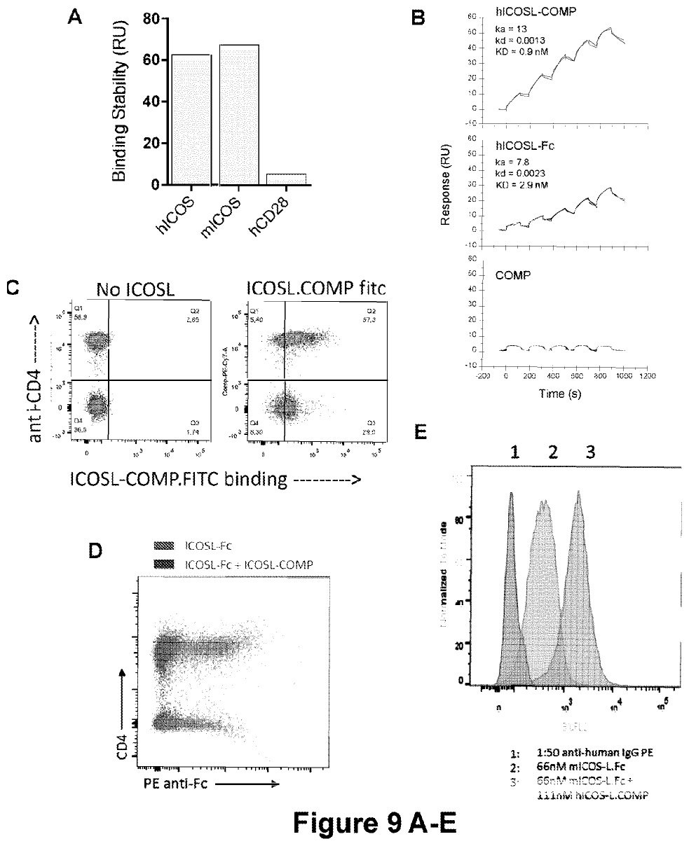

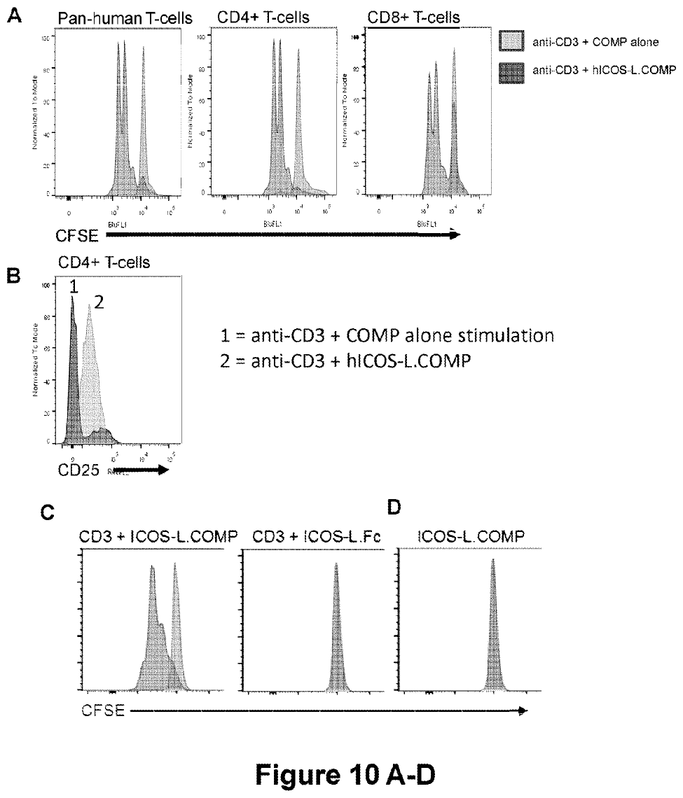

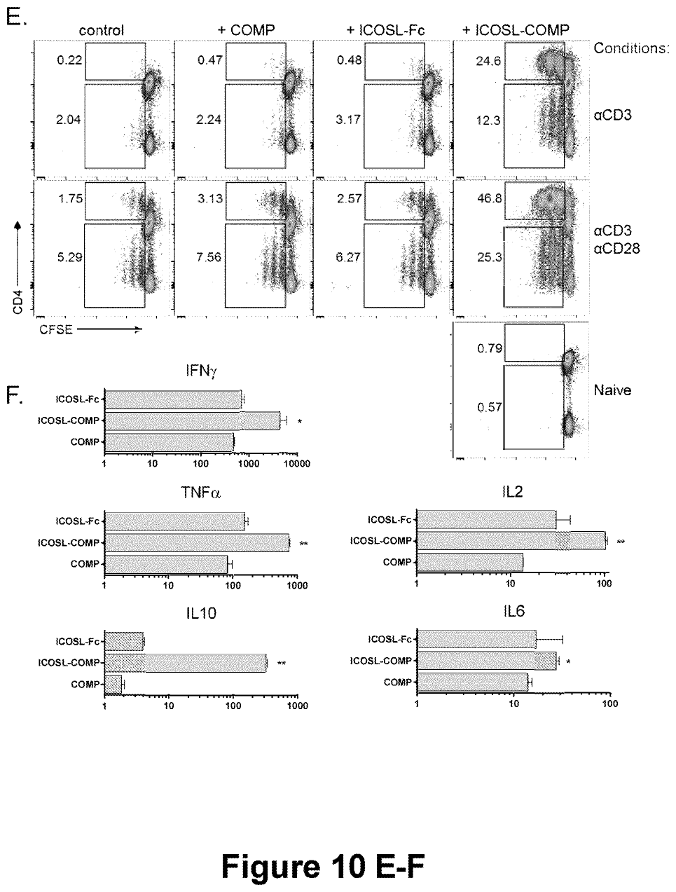

FIG. 1J shows decreased upregulation of the T-cell activation marker CD25 in CD3+CD4+ and CD8+ T-cells when cultured in ConA in the presence of VISTA.COMP compared to COMP or VISTA-Fc.

FIGS. 2 A-H illustrate that VISTA.COMP (FIG. 2 C, dark grey) binds to a clonal T-cell line (unstained control in white). and suppresses its activation (FIGS. 2A-2B).

FIG. 2A depicts 2.10 clonal T-cells that were activated in culture with immobilized anti-CD3 antibody (3 μg/mL) in the presence of immobilized or soluble VISTA.Fc or VISTA.COMP (10 μg/mL) and proliferation was measured by pulsing cells with 3H-Thymidine in the last 6 hours of a 24 hr culture. As observed with primary CD4+ T-cells, the pentameric VISTA.COMP suppressed proliferation both when immobilized or added soluble in culture media, whereas VISTA. Fc only exhibited suppressive activity when immobilized. (n=3, **p<0.01 relative to anti-CD3 stimulated control).

FIG. 2B depicts titration of soluble VISTA.COMP (dark grey) or VISTA.Fc (light grey) on 2.10 cells being activated as described in panel A. Data shows a lack of anti-proliferative function from cells treated with soluble VISTA. Fc at high concentrations (n=3, **p<0.01).

FIG. 2C depicts FACS analysis of biotinylated COMP, biotinylated VISTA.COMP, and VISTA.Fc (shaded histogram) binding to 2.10 clonal T-cells compared to unstained control (empty histograms).

FIG. 2D depicts anti-CD3 activated 2.10 T-cells that were treated with soluble VISTA. Fc or VISTA.COMP for 4 hours and the production of IL-2 measured by ICFC. Only VISTA.COMP significantly suppressed the number of IL-2 secreting cells (n=3, *p<0.05 relative to VISTA. Fc or non-treated).

FIG. 2E depicts 1×106 2.10 clonal T-cells that were cultured in a 6-well plate with immobilized anti-CD3 antibody and in the presence or absence of VISTA.COMP for 10 minutes. Proteins in complex with the T-cell receptor (TCR) were recovered by lysing the cells in each well and recovering the proteins adhered in each well (see solid phase immunoprecipitation (SPIP) in Methods). Recovered proteins were subsequently subject to immunoblot using anti-phosphotyrosine antibody (pY: clone 4G10). VISTA.COMP substantially diminished the phosphorylation of TCR complex proteins induced by TCR signalling.

FIGS. 2F-H depict stability of VISTA-Fc or VISTA.COMP binding to T-cell clones.

FIG. 2F depicts binding of VISTA-Fc or control-Fc (isotype control) to 2.10 T-cells, after one-step washing with FACS staining buffer.

FIG. 2G depicts binding of VISTA-Fc after two-step washing prior to FACS analysis. Loss of binding signal relative to A indicates a weak transient interaction of VISTA-Fc to the cell line.

FIG. 2H depicts binding of biotinylated VISTA.COMP to 2.10 cells and Jurkat cells is retained after two-step washing, indicating a more stable interaction of VISTA.COMP towards these cells.

FIGS. 3A-E illustrate that VISTA.COMP suppresses immune responses in-vivo.

FIG. 3A depicts a schematic representation of the skin allograft rejection model. On day 0, BALB/C animals were engrafted with skin from C57BL/6 mice and subsequently treated with VISTA.COMP (15 μg, I.V.) or PBS over the course of 15 days (arrows). Graft survival was monitored daily by a blinded investigator.

FIG. 3B depicts that treatment with VISTA.COMP significantly prolonged survival of skin allografts (n=6, *p<0.05 by Mann-Whitney U-Test).

FIG. 3C depicts treatment of VISTA.COMP 1 hr prior to Con-A injection rescued C57BL/6 mice from lethal hepatic injury at 24h (n=4).

FIGS. 3D and E show that survival correlated with a significant decrease in serum TNFα (n=5, *p<0.05) (FIG. 3D) and IL6 (n=5, *p<0.05) (FIG. 3E) 3 hours post Con-A injection.

FIGS. 4A-B illustrate the influence of two different tags on VISTA.COMP activity.

FIG. 5A is a schematic of B7-H4.COMP and PD-L1.COMP pentamers.

FIG. 5B is a SDS-PAGE gel of Ni.NTA-purified reduced and oxidized B7-H4.COMP, PD-L1.COMP, VISTA.COMP (+control) and COMP constructs expressed in Expi293F cells.

FIG. 6 shows that CFSE-labelled purified murine CD4+ T-cells were activated by plate-bound anti-CD3 antibody (2C11) in the presence of the indicated soluble ligand for 72 hours. FACS analyses of CFSE dilution revealed that PD-L1.COMP, B7-H4.COMP and VISTA.COMP all suppressed the expansion (top) and proliferation (bottom) of T cells relative to COMP only or no ligand (−).

FIG. 6 also shows T cells that unlike soluble dimeric VISTA.Fc, soluble VISTA.COMP can suppress T cell proliferation in response to CD3-TCR signaling.

FIG. 7A shows that IV injection of VISTA.COMP but not VISTA. Fc [every 3 days] blocks skin allograft rejection in mice until treatment was halted (asterisk).

FIG. 7B shows that treating C57BL/6 mice with VISTA.COMP by IP injection 1-hr prior to Con-A rescued 50% of them from lethal hepatic injury at 24h (n=13, p<0.05). VISTA. Fc was not effective. Moribund animals were classified as non-responders. Data was pooled from three independent trials. VISTA.COMP treated mice exhibited a significant decrease in serum TNFα (n=5, *p<0.05) and IL6 (n=5, *p<0.05) 3 hours post Con-A injection.

FIG. 8A is a schematic diagram of human ICOS-L.COMP pentamers.

FIG. 8B is a western blot and SDS-PAGE showing purity and molecular weight of the ICOS-L.COMP pentamers.

FIG. 9A shows the binding of hICOS-L.COMP to both human and mouse ICOS, but not CD28 as measured by Biacore T200 surface plasmon resonance experiments.

FIG. 9B shows characterization of ICOS.Fc binding to human ICOS-L.COMP, hICOSL-Fc, and COMP by surface plasmon resonance. Strong avidity of human ICOS-L.COMP for ICOS is reflected in the slow off-rate observed (kd) and low dissociation constant (0.9 nM), compared to 2.9 nM for ICOSL-Fc.

FIG. 9C shows FACS analyses demonstrating the binding of FITC-labelled ICOS-L.COMP binding to human CD3+CD4+ and CD3+CD4− T-cells.

FIG. 9D shows FACS analyses demonstrating the competitive displacement of hICOS-L.Fc binding to activated human CD3+ T-cells by human ICOS-L.COMP.

FIG. 9E shows FACS analyses demonstrating the competitive displacement of mICOSL-Fc binding to activated mouse CD4+ T-cells by hICOS-L.COMP.

FIG. 10A shows CFSE-based T cell proliferation assays that demonstrate the robust co-stimulation of both human CD4+ and CD8+ T-cells with soluble ICOS-L.COMP.

FIG. 10B shows a FACS analysis demonstrating that the activation of T cells with human ICOS-L.COMP results in the increased expression of CD25 on T cells (grey profile).

FIG. 10C shows CFSE-based T-cell proliferation demonstrating that soluble hICOS-L.COMP, but not ICOSL-Fc co-stimulates CD3+CD4+ human T-cells isolated from umbilical cord blood.

FIG. 10D shows a FACS analysis demonstrating that in the absence of anti-CD3 induced T-cell receptor signalling, ICOSL-COMP does not induce proliferation of human T-cells isolated from umbilical cord blood.

FIG. 10E shows CFSE T-cell proliferation assays demonstrating that soluble hICOSL-COMP, but not ICOSL-Fc co-stimulates CD3+CD4+ and CD3+CD4− T-cell proliferation in combination with anti-CD3 or anti-CD3/anti-CD28 induced signalling. T-cells were isolated from adult donor PBMCs.

FIG. 10F shows the cytokine secretion (IL2, IL6, IFNγ, TNFα, IL10) after 72 hours of stimulation of CD3+ T-cells (from adult PBMCs) with anti-CD3 in the presence of soluble COMP, ICOSL-COMP or ICOSL-Fc.

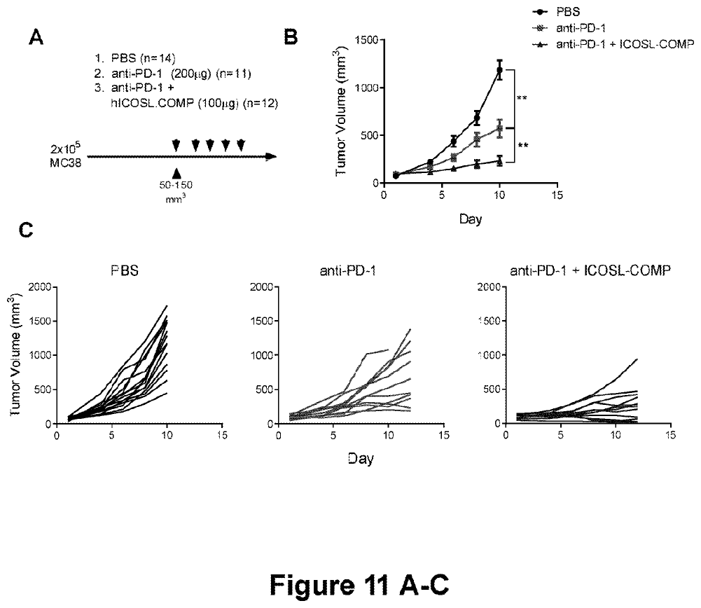

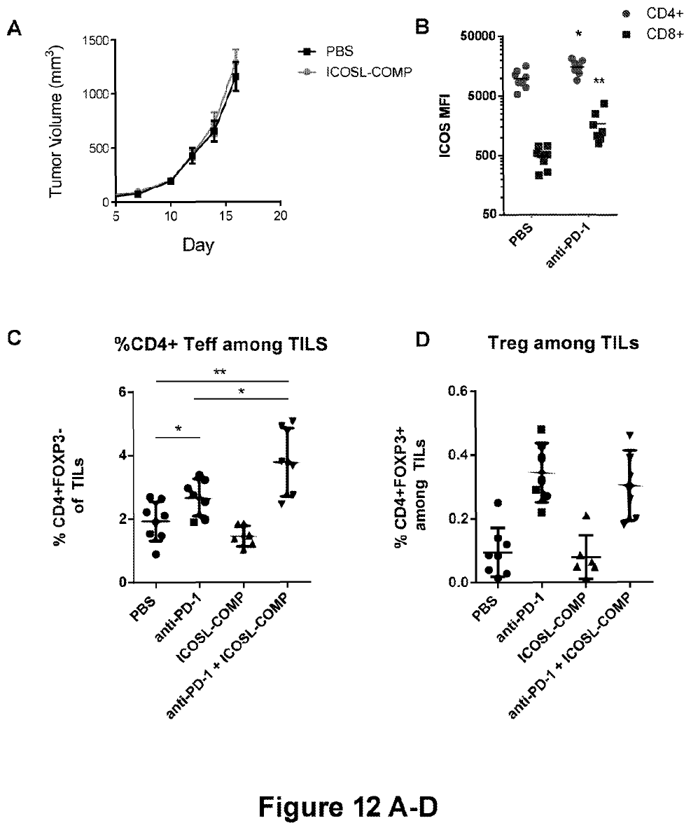

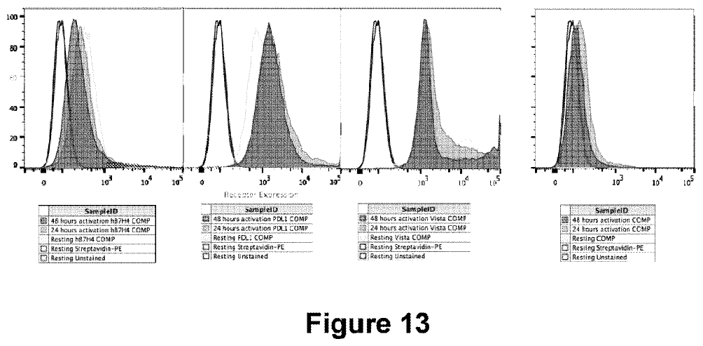

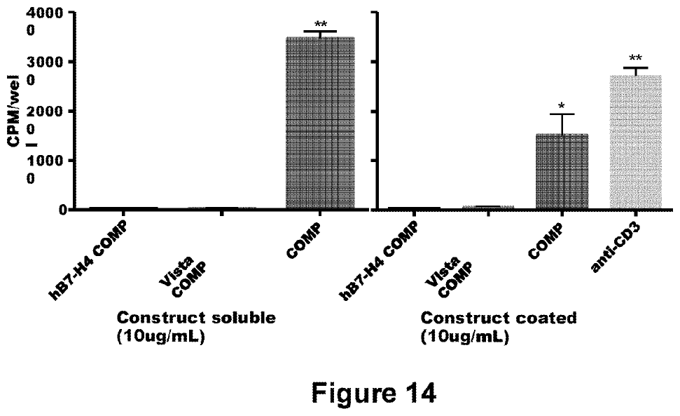

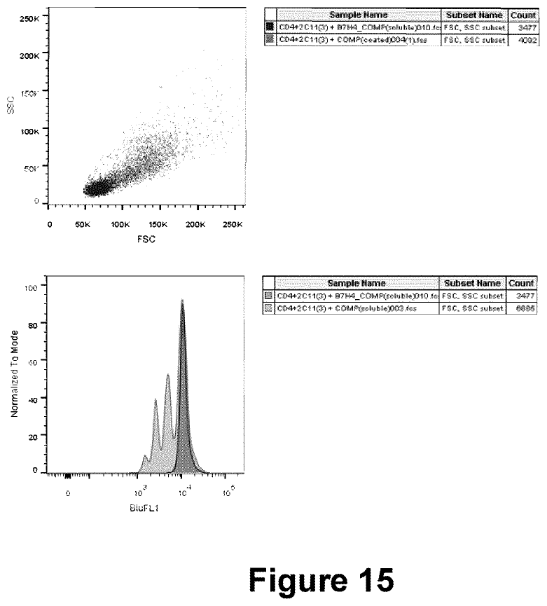

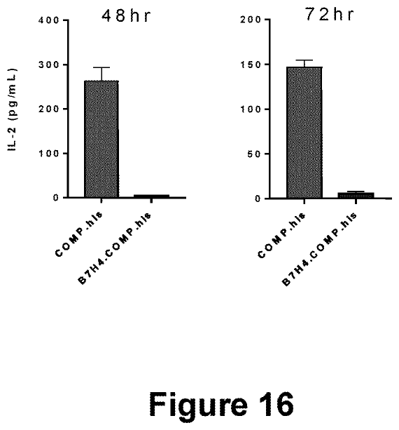

FIG. 11A is a schematic diagram detailing administration of compounds in in-vivo MC38 tumor model experiments.

FIG. 11B shows that ICOS-L.COMP acts in synergy with anti-PD-1 mAb to induce protective anti-tumor immunity in C57Bl/6 mice bearing established, subcutaneous murine colorectal MC38 tumor (therapeutic model).

FIG. 11C shows individual mice tumor volume profiles as a function of time.

FIG. 12A shows that ICOS-L.COMP monotherapy did not reduce the tumor growth in the MC38 tumor model.

FIG. 12B shows that anti-PD-1 treatment of MC38 tumor bearing animals leads to an increase in the expression of ICOS in intratumoral CD45+CD4+ and CD45+CD8+ T-cells.

FIG. 12C shows the TIL profiles in treated MC38 tumor bearing animals. The combination of anti-PD-1 and ICOS-L.COMP led to a significant increase in the abundance of CD45+CD4+FoxP3− cells among the TIL population.

FIG. 12D shows the TIL profiles in treated MC38 tumor bearing animals. The combination of anti-PD-1 and ICOS-L.COMP had no change in the abundance of CD45+CD4+FoxP3+ T-regulator cells among the TIL population.

FIG. 13 shows staining of 24h anti-CD3 activated or naive 2.10 T-cells with biotinylated PD-L1.COMP, B7-H4.COMP, or COMP. COMP shows negligible non-specific binding to this T-cell line compared to PD-L1 or B7-H4.COMP.

FIG. 14 shows that immobilized and soluble B7-H4.COMP suppresses the proliferation 2.10 T-cells undergoing activation with immobilized anti-CD3 antibody.

FIG. 15 shows that soluble B7-H4.COMP (10 ug/mL) substantially suppresses the expansion (top) and division (bottom) of primary murine CD4+ T-cells undergoing 72h of anti-CD3 induced activation in-vitro.

FIG. 16 shows that soluble B7-H4.COMP (10 ug/mL) inhibits IL-2 cytokine secretion from primary murine CD4+ T-cells undergoing 48h of anti-CD3 induced activation in-vitro.

### DETAILED DESCRIPTION OF THE NON-LIMITING EXEMPLARY EMBODIMENTS

Unless defined otherwise, all technical and scientific terms used herein generally have the same meaning as commonly understood by one of ordinary skill in the art to which this invention belongs.

V-domain Immunoglobulin suppressor of T-cell activation (VISTA) is a recently-discovered immune checkpoint ligand that functions to suppress T-cell activity. Other immune checkpoint ligands include B7-H4 and PD-L1. Activation of this immune checkpoint pathway in a subject has therapeutic potential, at least because it may reduce inflammatory responses in the subject by inhibiting T-cell activity. Conversely, ligands that stimulate T-cell activity, such as ICOS-L, have immune-boosting therapeutic potential, such as in cancer immunotherapy.

A dimeric construct of the IgV domain of VISTA (VISTA-Fc) was shown to suppress T-cell activation in-vitro. However, this effect required immobilization of VISTA-Fc to a solid substrate. Immobilization-dependent activity suggests that the efficacy of VISTA-Fc as a VISTA-receptor agonist in-vivo may be limited.

Provided herein is a pentameric polypeptide and monomers that make up same, each of the monomers containing an extracellular domain of VISTA, B7-H4, PD-L1 or ICOS-L genetically fused or linked to the pentamerization domain of cartilage oligomeric matrix protein (COMP).

COMP is a 524 kDa homopentamer of five subunits which consists of an N-terminal heptad repeat region (cc) followed by four epidermal growth factor (EGF)-like domains (EF), seven calcium-binding domains (T3) and a C-terminal globular domain (TC). The COMP pentamerization domain used herein is a 45-amino acid long sequence that spontaneously assembles into a bundle of 5 alpha-helices arranged in a parallel orientation and stabilized by disulphide bridges. Previously, a pro-angiogenic factor angiopoietin 1 that was fused to the COMP pentamerization domain (COMP-Ang1) showed increased stability relative to native Ang1, which lead to an increased induction of angiogenesis in-vivo21.

The inventors have generated engineered nucleic acids, engineered polypeptides, and engineered pentamerized polypeptides, human and murine, each of which include sequences of a VISTA, B7-H4, PD-L1 or ICOS-L extracellular domain operably linked to the pentamerization domain of COMP. By “extracellular domain” (or “ECD”), we mean the extracellular region of the polypeptide, or the nucleic acid that codes for it, that contains one or more Ig-type domains, which play a role in efficient binding between ligand and receptor. The ECD of VISTA, B7-H4, PD-L1 and ICOS-L comprises an IgV domain. The ECD of B7-H4, PD-L1 and ICOS-L also comprises an IgC domain. Engineered mRNAs corresponding to the engineered nucleic acids and/or polypeptides provided herein are also contemplated herein.

Appendix 1 provides nucleic acid and polypeptide sequences for use in preparing a VISTA, B7-H4, PD-L1 or ICOS-L extracellular domain operably linked to the pentamerization domain of COMP.

In an embodiment, a recombinant nucleic acid having a nucleic acid sequence encoding an extracellular domain of VISTA, B7-H4, PD-L1 or ICOS-L linked to a pentamerization domain of COMP is provided. In some embodiments, the nucleic acid encoding an extracellular IgV-containing domain VISTA has substantial similarity to SEQ ID NO: 1 (the human IgV-containing domain of VISTA) or SEQ ID NO: 2 (the mouse IgV-containing domain of VISTA). In some embodiments, the nucleic acid encoding the extracellular domain of B7-H4 has substantial similarity to SEQ ID NO: 26. In some embodiments, the nucleic acid encoding the extracellular domain of PD-L1 has substantial similarity to SEQ ID NO: 37. In some embodiments, the nucleic acid encoding the extracellular domain of ICOS-L has substantial similarity to SEQ ID NO: 48. In some embodiments, the nucleic acid encoding the pentamerization domain of COMP has substantial similarity to SEQ ID NO: 3 (the human pentamerization domain of COMP) or SEQ ID NO: 4 (the mouse pentamerization domain of COMP).

By “substantial similarity” in sequence, we mean sequences that are identical to or variants of the sequences provided herein, and encompass, or encode for a polypeptide that encompasses, the biological activity described herein.

For example, for nucleic acid sequences, substantially similar sequences include conservative variants that, because of the degeneracy of the genetic code, encode the amino acid sequence of one of the polypeptides provided herein. Variant nucleotide sequences include synthetically derived nucleotide sequences. Generally, variants of a particular nucleotide sequence of the invention will have at least at least 65%, 70%, 75%, 80%, 85%, 90%, 91%, 92%, 93%, 94%, 95%, 96%, 97%, 98%, 99%, or more sequence identity to one of the nucleotide sequences provided herein, as determined by sequence alignment programs known in the art, and using default parameters. In some preferred embodiments, substantially similar sequences are identical to the sequence referred to. In some preferred embodiments, the nucleic acid sequence is codon optimized for use in a genetic construct (e.g., for use in a plasmid).

Variant polypeptides encompassed by the present invention are biologically active, that is they continue to possess the biological activity of the pentamerized polypeptide described herein. Such variants may result from, for example, genetic polymorphism or from human manipulation. Biologically active variants of a polypeptide of the invention will have at least 75%, 80%, 85%, 90%, 91%, 92%, 93%, 94%, 95%, 96%, 97%, 98%, 99% or more sequence identity to one of the amino acid sequences provided herein as determined by sequence alignment programs known in the art using default parameters.

In an embodiment, the recombinant nucleic acid comprises SEQ ID NO: 1 operably linked to SEQ ID NO: 3 (i.e., the human IgV-containing domain of VISTA linked to the human pentamerization domain of COMP). In an embodiment, the recombinant nucleic acid comprises SEQ ID NO: 2 operably linked to SEQ ID NO: 4 (i.e., the mouse IgV-containing domain of VISTA linked to the mouse pentamerization domain of COMP).

A nucleic acid molecule is operably linked to another nucleic acid molecule when it is placed into a functional relationship with another nucleic acid molecule. For example, two nucleic acid molecules are operably linked when they are joined such that the amino acid sequences encoded by the two nucleic acid molecules provide for proper translation. Such nucleic acids may be operably linked through a linker sequence. Linker sequences suitable for use with the recombinant nucleic acids disclosed herein may be determined by a person of skill in the art. In some preferred embodiments, the linker sequence will be engineered to encode a somewhat flexible peptide or polypeptide (for example, it may be glycine rich).

In an aspect, a recombinant messenger ribonucleic acid (mRNA) having an mRNA sequence encoding an extracellular domain of VISTA linked to a pentamerization domain of COMP is provided. In some embodiments, the mRNA encoding the extracellular domain of VISTA has substantial similarity to SEQ ID NO: 5 (the human IgV-containing domain of VISTA) or SEQ ID NO: 6 (the mouse IgV-containing domain of VISTA). In some embodiments, the mRNA encoding the pentamerization domain of COMP has substantial similarity to SEQ ID NO: 7 (the human pentamerization domain of COMP) or SEQ ID NO: 8 (the mouse pentamerization domain of COMP).

In an embodiment, the recombinant mRNA comprises SEQ ID NO: 5 operably linked to SEQ ID NO: 7 (i.e., the human IgV-containing domain of VISTA linked to the human pentamerization domain of COMP). In an embodiment, the recombinant nucleic acid comprises SEQ ID NO: 6 operably linked to SEQ ID NO: 8 (i.e., the mouse IgV-containing domain of VISTA linked to the mouse pentamerization domain of COMP). Linker sequences suitable for use with the recombinant nucleic acids disclosed herein may be determined by a person of skill in the art. In some preferred embodiments, the linker sequence will be engineered to encode a somewhat flexible peptide or polypeptide (for example, it may be glycine rich).

In an aspect, a recombinant messenger ribonucleic acid (mRNA) having an mRNA sequence encoding an extracellular domain of B7-H4 linked to a pentamerization domain of COMP is provided. In some embodiments, the mRNA encoding the extracellular domain of B7-H4 has substantial similarity to SEQ ID NO: 27 (the human ECD of B7-H4). In some embodiments, the mRNA encoding the pentamerization domain of COMP has substantial similarity to SEQ ID NO: 7 (the human pentamerization domain of COMP).

In an aspect, a recombinant messenger ribonucleic acid (mRNA) having an mRNA sequence encoding an extracellular domain of PD-L1 linked to a pentamerization domain of COMP is provided. In some embodiments, the mRNA encoding the extracellular domain of VISTA has substantial similarity to SEQ ID NO: 62 (the human ECD of PD-L1). In some embodiments, the mRNA encoding the pentamerization domain of COMP has substantial similarity to SEQ ID NO: 7 (the human pentamerization domain of COMP).

In an aspect, a recombinant messenger ribonucleic acid (mRNA) having an mRNA sequence encoding an extracellular domain of ICOS-L linked to a pentamerization domain of COMP is provided. In some embodiments, the mRNA encoding the extracellular domain of VISTA has substantial similarity to SEQ ID NO: 61 (the human ECD of ICOS-L). In some embodiments, the mRNA encoding the pentamerization domain of COMP has substantial similarity to SEQ ID NO: 7 (the human pentamerization domain of COMP).

In an aspect, a recombinant polypeptide having an amino acid sequence encoding an extracellular domain of VISTA, B7-H4, PD-L1 or ICOS-L linked to a pentamerization domain of COMP is provided. In some embodiments, the amino acid sequence encoding the extracellular IgV-containing domain of VISTA has substantial similarity to SEQ ID NO: 9 (the human IgV-containing domain of VISTA) or SEQ ID NO: 10 (the mouse IgV-containing domain of VISTA). In some embodiments, the amino acid sequence encoding the extracellular domain of B7-H4 has substantial similarity to SEQ ID NO: 25. In some embodiments, the amino acid sequence encoding the extracellular domain PD-L1 has substantial similarity to SEQ ID NO: 36. In some embodiments, the amino acid sequence encoding the extracellular domain ICOS-L has substantial similarity to SEQ ID NO: 49. In some embodiments, the amino acid encoding the pentamerization domain of COMP has substantial similarity to SEQ ID NO: 11 (the human pentamerization domain of COMP) or SEQ ID NO: 12 (the mouse pentamerization domain of COMP).

As used herein, “link” means covalently or non-covalently associating one polypeptide to another polypeptide, regardless of the method of association. In one embodiment the association is a covalent association, such as a peptide bond. For example, a peptide having an amino acid sequence encoding an extracellular domain of VISTA, B7-H4, PD-L1 or ICOS-L of the invention can be linked to a pentamerization domain of COMP of the invention. Examples of such linkers are known in the art and are described for example, in Chen et. al (Reference 38). In an embodiment, an extracellular domain of VISTA, B7-H4, PD-L1 or ICOS-L of the invention can be linked to a pentamerization domain of COMP by forming a fusion protein between the extracellular domain of VISTA, B7-H4, PD-L1 or ICOS-L and the pentamerization domain of COMP. Such fusions proteins can be produced in host cells using expression vectors encoding VISTA, B7-H4, PD-L1 or ICOS-L ECDs and the pentamerization domain of COMP according to standard methods known in the art and as described herein.

In an embodiment, the recombinant polypeptide comprises SEQ ID NO: 9 operably linked to SEQ ID NO: 11 (i.e., the human extracellular IgV-containing domain of VISTA linked to the human pentamerization domain of COMP). In an embodiment, the recombinant nucleic acid comprises SEQ ID NO: 10 operably linked to SEQ ID NO: 12 (i.e., the mouse extracellular IgV-containing domain of VISTA linked to the mouse pentamerization domain of COMP). Linker sequences suitable for use with the recombinant nucleic acids disclosed herein may be determined by a person of skill in the art. In some preferred embodiments, the linker sequence will be engineered to encode a somewhat flexible peptide or polypeptide (for example, it may be glycine rich).

Herein, in some embodiments, the recombinant polypeptide may also be referred to as a recombinant protein, an engineered protein, or a fusion protein. By “fusion protein”, we mean a protein generated by joining two or more genes which originally coded for separate polypeptides. Translation of this fusion gene results in a single polypeptide with functional properties derived from each of the original polypeptides.

In some embodiments the nucleic acid or polypeptide of the invention may include an N-terminal leader sequence to enable secretion of the recombinant protein and/or a Histidine or other affinity tag for purification purposes. Methods for introducing an N-terminal leader sequence and/or a Histidine or other affinity tag are known in the art.

In an embodiment, the recombinant polypeptide is provided in a soluble form. As used herein, “soluble” means without immobilization on a solid substrate or a solid surface. In an embodiment, the activity of the recombinant polypeptide is substrate immobilization-independent (i.e., activity does not depend on the recombinant polypeptide being immobilized on a solid substrate or solid surface).

In an embodiment, an expression vector comprising a recombinant polypeptide disclosed herein is provided. In some embodiments, the expression vector further comprises at least one control sequence. By “control sequences”, we mean one or more sequences necessary for the expression of an operably linked coding sequence in a particular host organism. The control sequences that are suitable include, for example, promoters, polyadenylation signals, and/or enhancers. Methods and tools for generating an expression vector housing a recombinant polypeptide are known in the art and may be suitable for generating the expression vectors provided herein.

In an aspect, a host cell comprising an expression vector disclosed herein is provided. For example, the host cell may be HEK-293 or a HEK-293 derivative, CHO or a CHO derivative, or NS01 or an NS01 derivative. Methods and tools for generating a host cell housing an expression vector are known in the art and may be suitable for generating the host cells provided herein. In an embodiment, an engineered cell line comprising VISTA and COMP, B7-H4 and COMP, PD-L1 and COMP, or ICOS-L and COMP genetic material (as provided herein) integrated into the genome thereof is provided.

In an aspect, a pentamerized polypeptide having T-cell modulating activity is provided. The pentamerized polypeptide includes five monomers, each of the monomers comprising: a polypeptide having substantial similarity to an extracellular domain of a V-domain Ig Suppressor of T cell Activation (VISTA) (SEQ ID NO: 9 or 10); a polypeptide having substantial similarity to an extracellular domain of B7-H4 (SEQ ID NO: 25); a polypeptide having substantial similarity to an extracellular domain of PD-L1 (SEQ ID NO: 36); or a polypeptide having substantial similarity to an extracellular domain of ICOS-L (SEQ ID NO: 49); linked to a polypeptide having substantial similarity to a pentamerization domain of cartilage oligomeric matrix protein (COMP) (SEQ ID NO: 11 or 12).

In an embodiment, the pentamerized polypeptide comprises SEQ ID NO: 9 operably linked to SEQ ID NO: 11.

In an embodiment, the pentamerized polypeptide comprises SEQ ID NO: 10 operably linked to SEQ ID NO: 12.

In some embodiments, the pentamerized polypeptide is in a soluble form. In contrast to the dimerized VISTA-Fc, the pentamerized VISTA.COMP, B7-H4.COMP and PD-L1.COMP provided herein is biologically active in its soluble form. A skilled person will appreciate the possible advantages of an agent that is biologically active in its soluble relative to one that is active only in its immobilized form. For example, a soluble VISTA-receptor agonist, B7-H4-receptor agonist or PD-1 receptor agonist may exhibit increased activity in vivo, relative to a dimeric version (VISTA-Fc, B7-H4-Fc or PD-L1-Fc) which may require binding and clustering on accessory cells to induce immunosuppression.

In some embodiments, the soluble form pentamerized polypeptide has increased T-cell inhibitory activity relative to a soluble dimerized polypeptide comprising the IgV-containing domain of VISTA (SEQ ID NO: 9 or 10) (e.g., relative to VISTA-Fc) or relative to a soluble dimerized polypeptide comprising the ECD of B7-H4 (e.g., relative to B7-H4-Fc), or relative to a soluble dimerized polypeptide comprising the ECD of PD-L1 (e.g., relative to PD-L1-Fc). The T-cell inhibitory activity of the soluble form pentamerized polypeptide may be increased by 5%, 10%, 15%, 20%, 25%, 30%, 35%, 40%, 45%, 50%, 60%, 65%, 70%, 75%, 80%, 85%, 90% or 95% relative to the T-cell inhibitory activity of a soluble dimerized polypeptide comprising the IgV-containing domain of VISTA (SEQ ID NO: 9 or 10) (e.g., relative to VISTA-Fc) or relative to a soluble dimerized polypeptide comprising the ECD of B7-H4 (e.g., relative to B7-H4-Fc), or relative to a soluble dimerized polypeptide comprising the ECD of PD-L1 (e.g., relative to PD-L1-Fc). In some embodiments, the increased T-cell inhibitory activity comprises one or more of increased inhibition of T-cell activation and T-cell proliferation. Methods for determining T-cell inhibitory activity, T-cell activation and T-cell proliferation are known in the art and are described, for example, herein.

In some embodiments, the soluble form VISTA.COMP, B7-H4.COMP or PD-L1.COMP pentamerized polypeptide has increased immune inhibitory activity in vivo relative to a soluble dimerized polypeptide comprising an extracellular domain of VISTA (SEQ ID NO: 9 or 10) (e.g., relative to VISTA-Fc) or relative to a soluble dimerized polypeptide comprising the ECD of B7-H4 (e.g., relative to B7-H4-Fc), or relative to a soluble dimerized polypeptide comprising the ECD of PD-L1 (e.g., relative to PD-L1-Fc). The immune inhibitory activity of the soluble form pentamerized polypeptide may be increased by 5%, 10%, 15%, 20%, 25%, 30%, 35%, 40%, 45%, 50%, 60%, 65%, 70%, 75%, 80%, 85%, 90% or 95% relative to the immune inhibitory activity of a soluble dimerized polypeptide comprising the IgV-containing domain of VISTA (SEQ ID NO: 9 or 10) (e.g., relative to VISTA-Fc) or relative to a soluble dimerized polypeptide comprising the ECD of B7-H4 (e.g., relative to B7-H4-Fc), or relative to a soluble dimerized polypeptide comprising the ECD of PD-L1 (e.g., relative to PD-L1-Fc). For example, the increased immune inhibitory activity may include one or more of increased inhibition of cytokine secretion (e.g., IL-2 and/or IFNγ) and increased inhibition of cytotoxic lymphocyte (CTL) production. Methods for determining immune inhibitory activity, cytokine secretion and inhibition of cytotoxic lymphocyte (CTL) production are known in the art and are described, for example, herein. For example, the increased immune inhibitory activity may include suppression of inflammatory responses in-vivo, as demonstrated in the Examples section by data indicating prolongation of murine skin allograft survival, and protection of mice from lethal acute hepatitis.

In some embodiments, the soluble form ICOS-L.COMP pentamerized polypeptide has increased T-cell stimulatory activity relative to a soluble dimerized polypeptide comprising the ECD of ICOS-L (SEQ ID NO: 49) (e.g., relative to ICOS-L-Fc). The T-cell stimulatory activity of the soluble form pentamerized polypeptide may be increased by 5%, 10%, 15%, 20%, 25%, 30%, 35%, 40%, 45%, 50%, 60%, 65%, 70%, 75%, 80%, 85%, 90% or 95% or by 1, 2, 3, 4, 5, 6, 7, 8, 9, 10, 15, 20, 25, 30, 35, 40, 45, 50, 55, 60, 65, 70, 75, 80, 85, 90, 95 or 100 or more fold relative to the T-cell stimulatory activity of a soluble dimerized polypeptide comprising the ECD of ICOS-L (SEQ ID NO: 49) (e.g., relative to ICOS-L-Fc). In some embodiments, the increased T-cell stimulatory activity comprises one or more of increased stimulation of T-cell activation and T-cell proliferation. Methods for determining T-cell stimulatory activity, T-cell activation and T-cell proliferation are known in the art and are described, for example, herein.

In some embodiments, the soluble form ICOS-L.COMP pentamerized polypeptide has increased immune stimulatory activity in vivo relative to a soluble dimerized polypeptide comprising an extracellular domain of ICOS-L (SEQ ID NO: 49) (e.g., relative to ICOS-L-Fc). The immune stimulatory activity of the soluble form pentamerized polypeptide may be increased by 5%, 10%, 15%, 20%, 25%, 30%, 35%, 40%, 45%, 50%, 60%, 65%, 70%, 75%, 80%, 85%, 90% or 95% or by 1, 2, 3, 4, 5, 6, 7, 8, 9, 10, 15, 20, 25, 30, 35, 40, 45, 50, 55, 60, 65, 70, 75, 80, 85, 90, 95 or 100 or more fold relative to the immune stimulatory activity of a soluble dimerized polypeptide comprising the ECD of ICOS-L (SEQ ID NO: 49) (e.g., relative to ICOS-L-Fc). For example, the increased immune stimulatory activity may include one or more of increased cytokine secretion and increased cytotoxic lymphocyte (CTL) production. Methods for determining immune stimulatory activity, cytokine secretion and cytotoxic lymphocyte (CTL) production are known in the art and are described, for example, herein.

In some embodiments, the soluble form ICOS-L.COMP pentamerized polypeptide has an increase in the effector T-cells:regulatory T-cell ratios relative to a soluble dimerized polypeptide comprising an extracellular domain of ICOS-L (SEQ ID NO: 49) (e.g., relative to ICOS-L-Fc). The effector T-cells:regulatory T-cell ratios of the soluble form pentamerized polypeptide may be increased by 5%, 10%, 15%, 20%, 25%, 30%, 35%, 40%, 45%, 50%, 60%, 65%, 70%, 75%, 80%, 85%, 90% or 95% or by 1, 2, 3, 4, 5, 6, 7, 8, 9, or 10 fold relative to an appropriate control. Methods for determining effector T-cells:regulatory T-cell ratios are known in the art and are described, for example, herein.

In some embodiments, the soluble form of the VISTA.COMP, B7-H4.COMP or PD-L1.COMP pentamerized polypeptide provided herein exhibits activity as an agonist of the putative VISTA receptor, the putative B7-H4 receptor or the PD-1 receptor, respectively, in vitro and/or in vivo. By “agonist”, we mean an agent that binds to a receptor and activates the receptor thereby effecting a biological response.

In an embodiment, a pharmaceutical composition comprising one or more of the polypeptides, host cells, or pentamerized polypeptides disclosed herein and a pharmaceutically acceptable carrier, diluent, or excipient is provided herein.

The polypeptides or VISTA.COMP, B7-H4.COMP or PD-L1.COMP pentamerized polypeptides of the invention can be formulated in various ways using art recognized techniques. In some embodiments, the therapeutic compositions of the invention can be administered neat or with a minimum of additional components while others may optionally be formulated to contain suitable pharmaceutically acceptable carriers. As used herein, “pharmaceutically acceptable carriers” comprise excipients, vehicles, adjuvants and diluents that are well known in the art and can be available from commercial sources for use in pharmaceutical preparation (see, e.g., Gennaro (2003) Remington: The Science and Practice of Pharmacy with Facts and Comparisons: Drugfacts Plus, 20th ed., Mack Publishing; Ansel et al. (2004) Pharmaceutical Dosage Forms and Drug Delivery Systems, 7th ed., Lippencott Williams and Wilkins; Kibbe et al. (2000) Handbook of Pharmaceutical Excipients, 3rd ed., Pharmaceutical Press.)

Suitable pharmaceutically acceptable carriers comprise substances that are relatively inert and can facilitate administration of polypeptides, host cells or pentamerized polypeptides or can aid processing of the polypeptides, host cells or pentamerized polypeptides into preparations that are pharmaceutically optimized for delivery to the site of action.

Such pharmaceutically acceptable carriers include agents that can alter the form, consistency, viscosity, pH, tonicity, stability, osmolarity, pharmacokinetics, protein aggregation or solubility of the formulation and include buffering agents, wetting agents, emulsifying agents, diluents, encapsulating agents and skin penetration enhancers. Certain non-limiting examples of carriers include saline, buffered saline, dextrose, arginine, sucrose, water, glycerol, ethanol, sorbitol, dextran, sodium carboxymethyl cellulose and combinations thereof. Polypeptides, host cells or pentamerized polypeptides for systemic administration may be formulated for enteral, parenteral or topical administration. In certain embodiments the disclosed compositions will be formulated for intravenous administration and will preferably be infused using an IV container (e.g. an IV drip bag). Indeed, all three types of formulation may be used simultaneously to achieve systemic administration of the active ingredient. Excipients as well as formulations for parenteral and nonparenteral drug delivery are set forth in Remington: The Science and Practice of Pharmacy (2000) 20th Ed. Mack Publishing.

In an aspect, a method of eliciting a biological response in an individual in need thereof is provided. The method involves administering to the individual a therapeutically effective amount of: a VISTA-COMP fusion polypeptide (VISTA.COMP) comprising a) SEQ ID NO: 9 operably linked to SEQ ID NO: 11, or b) SEQ ID NO: 10 operably linked to SEQ ID NO: 12; a B7-H4-COMP fusion polypeptide (B7-H4.COMP) comprising SEQ ID NO: 25 operably linked to SEQ ID NO: 11; or a PD-L1-COMP fusion polypeptide (PD-L1.COMP) comprising SEQ ID NO: 36 operably linked to SEQ ID NO: 11. In this method, the biological response is one or more of: suppression of T-cell activation; suppression of T-cell proliferation; decreased secretion by T-cells of one or more inflammatory cytokines; suppressed induction of cytotoxic T lymphocytes (CTLs); and an increase in T-cells with regulatory phenotypes.

The suppression of T-cell activation in the individual administered the soluble form VISTA.COMP, B7-H4.COMP or PD-L1.COMP pentamerized polypeptide may be 5%, 10%, 15%, 20%, 25%, 30%, 35%, 40%, 45%, 50%, 60%, 65%, 70%, 75%, 80%, 85%, 90% or 95% relative to an appropriate control (for example, an individual receiving no polypeptide or a subject receiving soluble dimerized polypeptide). The suppression of T-cell proliferation in the individual administered the soluble form pentamerized polypeptide may be 5%, 10%, 15%, 20%, 25%, 30%, 35%, 40%, 45%, 50%, 60%, 65%, 70%, 75%, 80%, 85%, 90% or 95% relative to an appropriate control (for example, an individual receiving no polypeptide or a subject receiving soluble dimerized polypeptide). The secretion by T-cells of one or more inflammatory cytokines in the individual administered the soluble form pentamerized polypeptide may be decreased by 5%, 10%, 15%, 20%, 25%, 30%, 35%, 40%, 45%, 50%, 60%, 65%, 70%, 75%, 80%, 85%, 90% or 95% relative to an appropriate control (for example, an individual receiving no polypeptide or a subject receiving soluble dimerized polypeptide). The induction of cytotoxic T lymphocytes (CTLs) in the individual administered the soluble form pentamerized polypeptide may be suppressed by 5%, 10%, 15%, 20%, 25%, 30%, 35%, 40%, 45%, 50%, 60%, 65%, 70%, 75%, 80%, 85%, 90% or 95% relative to an appropriate control (for example, an individual receiving no polypeptide or a subject receiving soluble dimerized polypeptide). The increase in T-cells with regulatory phenotypes in the individual administered the soluble form pentamerized polypeptide may be increased by 5%, 10%, 15%, 20%, 25%, 30%, 35%, 40%, 45%, 50%, 60%, 65%, 70%, 75%, 80%, 85%, 90% or 95% or by 1, 2, 3, 4, 5, 6, 7, 8, 9, or 10 fold relative to an appropriate control (for example, an individual receiving no polypeptide or a subject receiving soluble dimerized polypeptide).

Methods for determining suppression of T-cell activation; suppression of T-cell proliferation; decreased secretion by T-cells of one or more inflammatory cytokines; suppressed induction of cytotoxic T lymphocytes (CTLs); and an increase in T-cells with regulatory phenotypes are known in the art and are described, for example, herein.

In an embodiment, a method of eliciting a biological response in an individual in need thereof is provided. The method involves administering to the individual a therapeutically effective amount of: an ICOS-L-COMP fusion polypeptide (ICOS-L.COMP) comprising SEQ ID NO: 49 operably linked to SEQ ID NO: 11. In this method, the biological response is one or more of: enhancement of T-cell activation; enhancement of T-cell proliferation; increased secretion by T-cells of one or more inflammatory cytokines; and enhanced induction of cytotoxic T lymphocytes (CTLs), and increases in the effector T-cells:regulatory T-cell ratios.

T-cell activation in the individual administered the soluble form ICOS-L.COMP pentamerized polypeptide may be increased or enhanced by 5%, 10%, 15%, 20%, 25%, 30%, 35%, 40%, 45%, 50%, 60%, 65%, 70%, 75%, 80%, 85%, 90% or 95% or by 1, 2, 3, 4, 5, 6, 7, 8, 9, 10, 15, 20, 25, 30, 35, 40, 45, 50, 55, 60, 65, 70, 75, 80, 85, 90, 95 or 100 or more fold relative to an appropriate control (for example, an individual receiving no polypeptide or a subject receiving soluble dimerized polypeptide (e.g., ICOS-L-Fc). T-cell proliferation in the individual administered the soluble form pentamerized polypeptide may be increased or enhanced by 5%, 10%, 15%, 20%, 25%, 30%, 35%, 40%, 45%, 50%, 60%, 65%, 70%, 75%, 80%, 85%, 90% or 95% or by 1, 2, 3, 4, 5, 6, 7, 8, 9, 10, 15, 20, 25, 30, 35, 40, 45, 50, 55, 60, 65, 70, 75, 80, 85, 90, 95 or 100 or more fold relative to an appropriate control (for example, an individual receiving no polypeptide or a subject receiving soluble dimerized polypeptide). The secretion by T-cells of one or more inflammatory cytokines in the individual administered the soluble form pentamerized polypeptide may be increased by 5%, 10%, 15%, 20%, 25%, 30%, 35%, 40%, 45%, 50%, 60%, 65%, 70%, 75%, 80%, 85%, 90% or 95% or by 1, 2, 3, 4, 5, 6, 7, 8, 9, 10, 15, 20, 25, 30, 35, 40, 45, 50, 55, 60, 65, 70, 75, 80, 85, 90, 95 or 100 or more fold relative to an appropriate control (for example, an individual receiving no polypeptide or a subject receiving soluble dimerized polypeptide). The induction of cytotoxic T lymphocytes (CTLs) in the individual administered the soluble form pentamerized polypeptide may be increased or enhanced by 5%, 10%, 15%, 20%, 25%, 30%, 35%, 40%, 45%, 50%, 60%, 65%, 70%, 75%, 80%, 85%, 90% or 95% or by 1, 2, 3, 4, 5, 6, 7, 8, 9, or 10 fold relative to an appropriate control (for example, an individual receiving no polypeptide or a subject receiving soluble dimerized polypeptide). The increase in the effector T-cells:regulatory T-cell ratios in the individual administered the soluble form pentamerized polypeptide may be increased by 5%, 10%, 15%, 20%, 25%, 30%, 35%, 40%, 45%, 50%, 60%, 65%, 70%, 75%, 80%, 85%, 90% or 95% or by 1, 2, 3, 4, 5, 6, 7, 8, 9, or 10 fold relative to an appropriate control (for example, an individual receiving no polypeptide or a subject receiving soluble dimerized polypeptide).

Methods for determining enhancement of T-cell activation; enhancement of T-cell proliferation; increased secretion by T-cells of one or more inflammatory cytokines; and enhanced induction of cytotoxic T lymphocytes (CTLs), and increases in the effector T-cells:regulatory T-cell ratios are known in the art and are described, for example, herein.

By “therapeutically effective amount”, we mean an amount effective to achieve the intended purpose (i.e., an amount sufficient to elicit a biological response in an individual in need thereof). Determination of a therapeutically effective amount is well within the capability of those skilled in the art.

The ICOS-L.COMP polypeptide may administered in combination with a checkpoint blocking molecule. As used herein a checkpoint blocking molecule is an agent that is capable of blocking immunoinhibitory signals to improve anti-tumor immune responses. The ICOS-L.COMP polypeptide can be administered simultaneously with, or before, or after the checkpoint blocking molecule. In an embodiment, the checkpoint blocking molecule is an inhibitor—for example an antagonistic antibody against PD-1, PD-L1, CTLA-4, LAG3, VISTA or TIM3.

Desired outcomes of the disclosed combinations are quantified by comparison to a control or baseline measurement. As used herein, relative terms such as “improve,” “increase,” or “reduce” indicate values relative to a control, such as a measurement in the same individual prior to initiation of treatment described herein, or a measurement in a control individual (or multiple control individuals) in the absence of the soluble form pentamerized polypeptides described herein but in the presence of other therapeutic moiety(ies) such as standard of care treatment. A representative control individual is an individual afflicted with the same condition as the individual being treated.

Changes or improvements in response to therapy (whether additive or synergistic) may prove to be statistically significant. As used herein, the term “significance” or “significant” relates to a statistical analysis of the probability that there is a non-random association between two or more measured responses. To determine whether or not a relationship is “significant” or has “significance,” a “p-value” can be calculated. P-values that fall below a user-defined cut-off point are regarded as significant. For the purposes of the instant invention a p-value less than or equal to 0.1, less than 0.05, less than 0.01, less than 0.005, or less than 0.001 may be regarded as significant.

A synergistic therapeutic effect may be an effect of at least about two-fold greater than the therapeutic effect elicited by a single therapeutic moiety, or the sum of the therapeutic effects elicited by the single therapeutic moieties) of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single therapeutic, or the sum of the therapeutic effects elicited by the single therapeutic moieties of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

The particular dosage regimen, i.e., dose, timing and repetition, will depend on the individual subject, as well as empirical considerations such as pharmacokinetics (e.g., half-life, clearance rate, etc.). Determination of the frequency of administration may be made by persons skilled in the art, such as an attending physician based on considerations of the condition and severity of the condition being treated, age and general state of health of the subject being treated and the like. Frequency of administration may be adjusted over the course of therapy based on assessment of the efficacy of the selected composition and the dosing regimen. Such assessment can be made on the basis of markers of the specific disease, disorder or condition or assessments of the individuals wellbeing (as measured using quality of life assessments, activities of daily living, etc.).

### EXAMPLES

### Example 1: VISTA.COMP Methods

Recombinant Protein Expression and Purification

VISTA. Fc was produced by cloning a synthetic dsDNA coding for the extracellular domain (ECD; residues 16-194 of SEQ ID NO: 10) of murine VISTA upstream of the human IgG-1 Fc region (GeneArt; Thermo Fisher Scientific) into the pcDNA-3.4 expression plasmid (Thermo Fisher Scientific). The plasmid encoding the murine VISTA.COMP (SEQ ID NO: 14) gene was similarly generated by inserting a synthetic dsDNA coding for the ECD of VISTA, flanked by 5′ and 3′ EcoRI restriction digestion sites, upstream of the cartilage oligomeric matrix protein pentamerization domain (COMP; residues 28-72 of SEQ ID NO: 12) followed by a C-terminal hexahistidine tag. An expression plasmid coding for COMP domain alone (control) was constructed by excising the VISTA ECD region from the VISTA.COMP plasmid by EcoRI restriction digestion. All plasmids encoded a 5′ Ig-kappa leader sequence for high protein secretion in mammalian cells. Recombinant proteins were expressed using the Expi-293TM transient expression system (Thermo Fisher Scientific). Human VISTA.COMP (hVISTA.COMP, SEQ ID NO: 24) was created as above but with the exception of DNA encoding for the hVISTA ECD (SEQ ID NO: 9) in place of the mouse VISTA ECD and the human COMP sequence (SEQ ID NO:11) in place of the mouse COMP domain. Secreted VISTA. Fc was purified from culture media using HiTrap Protein A HP columns (GE Healthcare), while the histidine tagged VISTA.COMP and COMP were purified using Ni-NTA resin (Qiagen) and desalted into PBS pH 7.4 using PD-10 columns (GE Healthcare). Proteins were verified for purity using SDS-PAGE, and protein concentration quantified by BCA assay (Pierce) or A280 measurements.

Animals

C57BL/6 mice used throughout this study were housed in a pathogen free environment at the Sunnybrook Research Institute Comparative Research (SRICR) facility while CD200R1^{-/-} mice were bred at the Toronto Research Institute Animal facility. All protocols were approved by the SRICR animal care committee, accredited by the Canadian Council of Animal Care.

Cell Culture

CD4+ T-cells were isolated from spleens of C57Bl/6 mice using an EasySep Mouse CD4+ T-cell isolation kit (Stem Cell) and cultured in RPMI-1640 media supplemented with 10% FBS, penicillin (100U/mL), streptomycin (100 μg/mL) and 0.05 mM 2-mercaptoethanol. The murine 2.10 T-cell clone was cultured in complete IMDM supplemented with IL-2 (3.5 μg/mL), lecithin (20 μg/mL), and BSA (0.5 mg/mL).

2.10 T-Cell Clone Activation

96-well microtiter plates were coated with anti-CD3 antibody (3 μg/mL in PBS, clone 145-2C11, BioXcell) at 4° C. overnight. To monitor the effects of immobilized checkpoint ligands on 2.10 cell activation, the anti-CD3 coated wells were washed and coated with VISTA.COMP or other recombinant proteins for 1 hr at 37° C. in PBS. Wells were then washed with PBS (3×) to remove residual unbound proteins. Murine 2.10 T-cells grown in culture were recovered, washed in IMDM (×3), and dispended into protein-coated wells (1×10⁴ cells/well). To measure proliferation, cells were pulsed for 6 hr with 1 μCi of [³H]-Thymidine after 18 hr of culture and uptake quantified using a TopCount NXT scintillation counter (Perkin Elmer). To assay the sensitivity of 2.10 T-cells to soluble checkpoint ligands, indicated recombinant proteins were diluted in culture media and added to anti-CD3 antibody-coated wells simultaneously with the 2.10 cells.

CD4+ T-Cell Proliferation and Cytokine Secretion (Mouse)

Isolated murine CD4+ T-cells were labelled with CFSE following the manufacturers protocol (Thermo Fisher Scientific) and stimulated in 96-well microtiter plates pre-coated with an anti-CD3 antibody in the presence of either murine VISTA. Fc, VISTA.COMP or COMP alone (coated or soluble). Cells were harvested 48 or 72 hrs later and CFSE-dilution profiles quantified by flow cytometry (FACScalibur, Becton Dickinson). Culture media were harvested from stimulated CD4+ T-cells at 48 or 72 hr and analysed by enzyme linked immunosorbent assay (ELISA, R&D System) to quantify VISTA.COMP-mediated inhibition of IL2 and IFNγ secretion.

Human T-Cell Activation and Proliferation Assays

Peripheral blood mononuclear cells (PBMCs) isolated from healthy donors (STEM CELL Technologies) were cultured with 5 ug/mL ConA for 48 or 72 hours or immobilized anti-CD3 antibody (OKT3, 1 ug/mL) in the presence of VISTA.Fc, VISTA.COMP, or COMP. In some cases, cells were labelled before culture with CFSE to trace proliferation. After culture cells were harvested, stained with the indicated antibody (anti-CD3, anti-CD4, anti-CD8, and/or anti-CD25) and analysed by flow cytometry.

Flow Cytometry Binding Assays

Binding of VISTA.COMP, VISTA. Fc, or control proteins to T-cells was assessed using flow cytometry. Proteins were first biotinylated using EZ-Link Sulfo-NHS-LC-Biotin reagent (Thermo Scientific) as directed by the manufacturer. Upon completion of the reaction, the excess biotin was removed using a PD-10 (GE Healthcare) desalting column. To confirm equivalent levels of biotinylation of each protein, the quantity of biotin conjugated to each ligand was determined using HABA/Avidin reagent (Sigma). 2.10 T-cells were incubated with the indicated biotinylated protein (10 μg/100 μL) or VISTA.Fc for 0.5 hr at 4° C. in FACS staining buffer (PBS supplemented with 1% FBS and 0.09% NaN₃). After removal of non-bound proteins, cells were incubated with streptavidin-PE (1:300, BioLegend) or PE-anti-human IgG (1:100, BioLegend) in FACS staining buffer for 15 minutes and the PE-fluorescence signal analysed using a FACScalibur cell analyzer.

Allogeneic Mouse Mixed Leukocyte Culture Assay (Allo-MLC)

VISTA.COMP or CD200Fc (positive control) were added to allogeneic murine mixed leukocyte cultures for 5 days and induction of cytotoxic T-lymphocytes (CTLs) assayed as previously described (Reference 39). Briefly, C57Bl/6 responder splenocytes were incubated with an equal number of irradiated BALB/c stimulator cells in the presence of each recombinant protein at the indicated concentration. Induced CTLs were assayed by monitoring the release of ⁵¹Cr from loaded P815 mastocytoma target cells over 5 hrs (25:1 effector to target ratio).

Allogeneic Skin Graft Transplant

The immunosuppressive effect of VISTA.COMP was tested in-vivo using a mouse skin allograft model as previously described (Reference 39). BALB/C mice received C57Bl/6 skin grafts (day 0) followed by treatment with VISTA.COMP (15 μg IV) once every 3 days for a total of 5 treatments in combination with low-dose rapamycin (0.5 mg/kg, I.P. injections every 48 hrs). A blinded investigator monitored graft survival daily.

Concanavalin-A Induced Acute Hepatitis

The ability of VISTA.COMP to rescue mice from lethal acute inflammation was evaluated using the Con-A model of acute hepatitis. Male C57Bl/6 mice were treated I.P. with VISTA.COMP (200 μg) or PBS, two hours prior to I.V. injection of a lethal dose (15 mg/kg) of Con-A (Sigma-Aldrich). A subset of animals were sacrificed after 3 hours to quantify serum IL-6 and TNFα levels by ELISA (R&D Systems) and the remaining animals were monitored for survival over the course of 24 hours.

Solid Phase Immunoprecipitation Assay

A solid phase immunoprecipitation assay was performed to assess the inhibitory effects of VISTA.COMP on TCR phospho-signalling cascades. 2.10 T-cells were exposed to plates coated with anti-CD3 antibody (with or without VISTA.COMP) for 15 minutes. Residual medium was removed and cells lysed in situ upon incubation with lysis buffer (50 mM Tris pH 7.4, 150 mM NaCl, 1% NP40, 5 mM Na₄O₇P₂, 5 mM NaF, 2 mM Na₃VO₄, and 1× Sigma Protease Inhibitor Cocktail) for 30 minutes at 4° C. Wells were vigorously washed 3× with lysis buffer, and adhered proteins eluted with 3.5% NH₄OH. The eluted proteins were lyophilized and resuspended in SDS-sample buffer, and total phosphorylated proteins visualized by western blot using an anti-phosphotyrosine antibody (clone 4G10; Sunnybrook Antibody Core Facility).

Statistics

Statistical analyses were performed using GraphPad Prism software (v6.0.2) using either Students T-test, or Mann Whitney U-tests where indicated. Graphs and visuals were created using GraphPad Prism software.

### Example 2: VISTA.COMP Results

Dimeric VISTA Suppresses T-Cell Proliferation Only when Immobilized

The activation of immune checkpoint receptors on T-cells has, in some cases, been initiated through the binding of an IgV domain displayed by a protein ligand such as PD-L1 expressed on APCs and tumour cells to a complementary IgV domain of its cognate immune checkpoint receptor PD-1 on T-cells. Past studies have demonstrated that monomeric forms of these IgV domains involving PD1:PD-L1 and CD28:CD80/CD86 interact with each other with modest affinity, reflected by Kd values typically in the low micromolar (μM) range (References 40, 41). To activate checkpoint receptors on T-cells in-vitro, these immune checkpoint ligands have been expressed as oligomers, such as Fc fusion proteins, which have been immobilized on a surface. The immobilized presentation mimics avidity events taking place when such immune checkpoint domains are displayed on the surface of APCs and T-cells.

Consistent with previous reports (References 8, 9), a dimeric form of VISTA (VISTA-Fc), constructed by fusing the VISTA IgV domain with the Fc region from IgG1, suppressed the proliferation of anti-CD3 stimulated CD4+ T-cells only when it was immobilized on a culture dish (FIG. 1A). Soluble VISTA-Fc added to culture media during CD4+ T-cell stimulation had negligible impact on cell proliferation, suggesting that use of VISTA-Fc in-vivo to suppress T-cell activity may be limited due to its inability to fully agonize the putative VISTA-receptor.

Without being bound by theory, the lack of activity in soluble VISTA-Fc in-vitro may be caused by insufficient avidity towards its receptor and/or a lack of ability to cluster the VISTA-receptor on the cell surface.

A higher order VISTA oligomer was engineered in order to generate an agonist that may effectively suppress T-cell stimulation both in-vitro and in-vivo. A recombinant VISTA pentamer (VISTA.COMP; see SEQ ID NO: 14 of Appendix 1 for sequence) was constructed by genetically fusing the VISTA IgV domain to the COMP pentamerization domain. Recombinant VISTA.COMP was produced in a mammalian expression system, yielding a pentameric protein of ˜250 kDa stabilized by intramolecular disulphide bonds within the COMP pentamerization domain (FIG. 1B).

VISTA.COMP Suppresses T-Cell Activation and Proliferation as a Soluble Ligand In-Vitro

In contrast to VISTA-Fc, soluble VISTA.COMP substantially suppressed expansion and proliferation of isolated anti-CD3 stimulated CD4+ T-cells (FIG. 1C). The recombinant COMP domain alone showed negligible effect on T-cell expansion and proliferation suggesting that VISTA.COMP activity is due to VISTA signalling, and not off-target events associated with the COMP domain. In addition, soluble VISTA.COMP significantly diminished (p<0.01) the secretion of inflammatory cytokines IL-2 (FIG. 1D) and IFNγ (FIG. 1E) by stimulated CD4+ T-cells. The efficacy of VISTA.COMP suppression was inversely correlated with the strength of T-cell receptor (TCR) stimulation, as increased anti-CD3 stimulation led to increases in T-cell division in the presence of VISTA.COMP (FIG. 1F). In addition to its ability to suppress T-cell proliferation in response to a polyclonal stimulus, VISTA.COMP readily suppressed the induction of cytotoxic T-lymphocytes (CTLs) in a dose dependent manner in allogenic mixed-leukocyte cultures (FIG. 1G). These results demonstrate that VISTA.COMP is an effective agonist, capable of activating the VISTA-receptor on T-cells to regulate their activity. Unlike VISTA-Fc, VISTA.COMP does not require immobilization on a solid surface to exhibit agonistic activity.

hVISTA.COMP Suppresses the Activation and Proliferation of Human T-Cells

A human version of VISTA.COMP (SEQ ID NO: 24) was constructed as described for mVISTA.COMP with replacement of the mouse VISTA ECD with that of hVISTA.COMP, and the replacement of mouse COMP pentamerization domain with that of human COMP. This protein—hVISTA.COMP (SEQ ID NO: 24)—was readily expressed by Expi293F cells and purified to homogeneity (FIG. 1H). Similar to what was previously observed in experiments with mouse T-cells, hVISTA.COMP, but not COMP, was found to readily suppress the proliferation of human T-cells isolated from adult PBMCs induced by ConA (FIG. 11). Furthermore, hVISTA.COMP suppressed upregulation of the CD25 T-cell activation marker in human CD4+ and CD8+ T-cells undergoing anti-CD3 induced activation (FIG. 1J). Together these results indicate that similar to what was previously observed with mVISTA.COMP, human VISTA.COMP can induce VISTA-mediated immunoinhibitory signalling to suppress the activation of human T-cells.

VISTA.COMP Binds to a Clonal T-Cell Line and Suppresses its Activation

In addition to primary CD4+ T-cells, it was found that a CD4-negative murine IL-2 dependent T-cell clone (2.10) (Reference 42) was sensitive to VISTA inhibitory signalling, providing a controlled system to assay the effects of VISTA-receptor agonists. Consistent with what was observed in primary CD4+ T-cells, VISTA-Fc suppressed anti-CD3 induced proliferation only when immobilized on a solid surface, while VISTA.COMP suppressed activity when immobilized or when provided in a soluble form in culture media (p<0.01) (FIG. 2A). Titration of soluble VISTA.COMP and VISTA-Fc demonstrated that VISTA.COMP suppressed anti-CD3-induced 2.10 cell proliferation at concentrations as low as 1 μg/mL (p<0.01), whereas VISTA-Fc had no detectable activity at concentrations as high as 30 μg/mL (FIG. 2B). In addition to suppressing proliferation, intracellular flow cytometry showed that soluble VISTA.COMP, but not VISTA-Fc, suppressed stimulated 2.10 cell IL-2 secretion within 4 hours of exposure (p<0.05), suggesting an immediate and rapid effect of VISTA.COMP (FIG. 2D). VISTA.COMP suppressed the rapid phosphorylation of tyrosine residues within TCR-complex signalling proteins induced upon anti-CD3 stimulation of the 2.10 cells (FIG. 2E). Mechanistically, these results are consistent with the previous finding that exposing naïve CD4+ T-cells to immobilized VISTA-Fc, led to long-term suppression of T-cells upon transfer to anti-CD3 coated wells (in the absence of further VISTA-Fc), which suggests a role for VISTA signalling as an early regulator of T-cell activation⁹. Flow cytometry was then performed on the 2.10 cell line using VISTA-Fc, COMP, or VISTA.COMP, to determine if the inability of soluble VISTA-Fc to bind to the VISTA-receptor on T-cells contributes to the lack of suppressive activity. VISTA.COMP and COMP were labelled with an equivalent number of biotin groups, and cell-bound biotinylated proteins were detected with PE-streptavidin, while bound VISTA-Fc was detected with PE-anti-IgG. Both VISTA-Fc and VISTA.COMP were found to bind to naïve 2.10 T-cells while the baseline signal observed for COMP confirmed the absence of non-specific binding arising from the pentamerization domain alone (FIG. 2C). Unlike VISTA.COMP, the VISTA-Fc signal could be readily displaced by additional washing steps suggesting that its interaction with the putative VISTA receptor is of low affinity (FIGS. 2F-H). Altogether, these findings demonstrate that a soluble low-avidity VISTA ligand, such as VISTA-Fc, is not sufficient to activate immunoinhibitory signalling through this pathway in-vitro. Only the high-avidity VISTA.COMP ligand was capable of stimulating the VISTA-receptor.

VISTA.COMP Suppresses Immune Responses In-Vivo

In view of the data showing that VISTA.COMP suppresses T-cell activity in-vitro as a soluble ligand, it may be a useful agonist to suppress pro-inflammatory responses in-vivo. VISTA.COMP was first tested in a murine skin allograft model. BALB/C mice received non-histocompatible skin allografts (from C57Bl/6 donors) before receiving treatment with VISTA.COMP or a saline control combined with low-dose rapamycin (FIG. 3A). It was previously demonstrated that this dose of rapamycin has no effect on graft survival as a monotherapy (Reference 39). VISTA.COMP significantly prolonged the survival of skin allografts. Only 1/6 allografts were rejected in the VISTA.COMP treatment group at the last day of treatment (day 15) relative to 6/6 rejected allografts observed in the saline control group (p<0.05, Mann-Whitney U-test) (FIG. 3B). The immunosuppressive effects of VISTA.COMP were also evaluated in an acute inflammatory hepatic model called Concanavalin-A (ConA) induced hepatitis. In this model, administration of ConA induces acute liver inflammation mediated by a polyclonal activation of CD4+ T and NKT cells (Reference 43). This model was used to assess the suppressive activity of VISTA.COMP on T-cells in-vivo. Previous research in this model suggested that agonistic anti-VISTA antibodies directed towards VISTA on T-cells could rescue mice from lethal hepatic injury (Reference 10). However, the effect of treatment with VISTA-receptor agonist was unknown. It was found that prophylactic treatment of mice with VISTA.COMP rescued ¾ of male C57Bl/6 mice from succumbing to a lethal dose of ConA (FIG. 3C). It was consistently found that serum levels of TNFα and IL-6 at a 3-hour time point, post-ConA injection, were significantly reduced upon VISTA.COMP treatment (p<0.05) (FIG. 3D). The results from these two acute inflammatory disease models suggest that VISTA.COMP may serve as a strong agonist to suppress inflammatory responses in-vivo.

Influence of Tag on VISTA.COMP Activity

Proliferation of anti-CD3 stimulated CFSE-labelled splenic CD4+ T-cells in the presence of coated or soluble VISTA constructs (FIG. 4A). Soluble VISTA. Fc had no suppressive effects on the CD4+ T-cells, while VISTA.COMP exhibited immunosuppressive activity on the CD4+ T-cells without requiring immobilization. Further, histidine tagged VISTA.COMP (VISTA.COMP.his; SEQ ID NO: 14 in Appendix 1) exhibited increased activity relative to strep II tagged VISTA.COMP (VISTA.COMP.SS; SEQ ID NO: 60), suggesting an influence of tag on protein stability. Using SPR binding assays, a commercial anti-VISTA antibody (clone 730802) RnD systems) recognized VISTA.COMP.his but not VISTA.COMP.SS (FIG. 4B) suggesting a fundamental change in epitope exposure (+ indicates detectable binding by SPR, − indicated complete absence of binding).

VISTA.COMP is a High-Avidity Checkpoint Receptor Agonist

The data provided herein suggest that VISTA.COMP is a high-avidity checkpoint receptor agonist capable of suppressing T-cell activities in-vitro and capable of suppressing inflammatory responses in-vivo. Comparisons between immobilized and soluble VISTA-Fc and VISTA.COMP show that activity as a VISTA-receptor agonists is dependent on the level of oligomerization, the higher-avidity multimer created using the COMP pentamerization domain being required for activity in solution (i.e., in the absence of immobilization to a substrate). The inventors have found the COMP domain to be a useful scaffold for expressing stable VISTA pentamers. The data provided herein, combined with the observation of exacerbated autoimmune diseases observed upon genetic deletion of VISTA in mice, suggest a potential utility of targeting the VISTA-mediated immunosuppression pathway to clinically suppress undesired immune responses.

### Example 3: ICOS-L.COMP Methods

Recombinant ICOS-L.COMP Expression and Purification

A dsDNA construct was synthesized (GeneArt; Thermo Fisher Scientific) containing dsDNA encoding the human ICOS-L extracellular domain (ECD)(SEQ ID NO: 49) upstream of dsDNA encoding the human COMP pentamerization domain (SEQ ID NO: 11) with a C-terminal histidine tag and inserted in the pcDNA3.4 expression plasmid (GeneArt; Thermo Fisher Scientific). The sequence also contained the Ig-kappa leader sequence at the 5′ end of the ICOS-L ECD, and the nucleotide sequences were codon optimized to allow high yield secretion from human derived cell lines (ICOS-L.COMP; see SEQ ID NO: 57 of Appendix 1 for sequence). The ICOS-L.COMP encoding plasmid was transfected into Expi293F cells following manufacturers recommendations (GeneArt; Thermo Fisher Scientific) and a stable cell line secreting ICOS-L.COMP was selected by exposing the transfected cell to geneticin (GeneArt; Thermo Fisher Scientific) for two-weeks. Secreted histidine-tagged ICOS-L.COMP was purified from cell-culture supernatants using HisTrap HP columns (GE Healthcare). Subsequent purification, protein samples were desalted into PBS, pH 7.4, using PD10 columns (GE Healthcare). Proteins were verified for purity using SDS-PAGE, and protein concentration quantified by BCA assay (Pierce) or A280 measurements.

Animals

C57BL/6 mice used throughout this study were housed in a pathogen free environment at the Sunnybrook Research Institute Comparative Research (SRICR) facility. All protocols were approved by the SRICR animal care committee, accredited by the Canadian Council of Animal Care.

Human T-Cell Proliferation and Activation

The ability of ICOS-L.COMP or ICOSL-Fc (R&D Systems) to co-stimulate human T-cells in vitro as a soluble ligand was assayed. Human T-cells were isolated from Ficoll-Paque separated human cord blood cells or adult PBMCs using the EasySep Human T-cell Isolation Kit (STEMCELL Technologies). Isolated T-cells were labelled with CFSE following manufacturers protocol (Thermofisher) and stimulated in 96-well plates coated with an anti-CD3 antibody (clone OKT3, BioXcell). Cells in each well were cultured in RPMI-1640 media supplemented with 10% FBS, penicillin (100U/mL), streptomycin (100 μg/mL) and 0.05 mM 2-mercaptoethanol. Selected wells were incubated with soluble COMP or ICOS-L.COMP at titrated concentrations. Cells were harvested 48-72 hours later, stained with the appropriate antibody (anti-CD4, anti-CD8, and/or anti-CD25) and analysed by flow cytometry (FACScalibur, Becton Dickinson) for proliferation (CFSE) and upregulation of activation markers (i.e., CD25). Cell culture supernatants from these wells were also collected for cytokine analysis at 72 hr and secretion of IFNγ, TNFα, IL10, IL2 and IL6 quantified using the human LEGENDplex Th1 inflammation panel (Biolegend).

ICOS-L.COMP Binding to hICOS, mICOS, and CD28

Direct binding of ICOS-L.COMP to hICOS, mICOS, and hCD28 was evaluated using Biacore T-200 surface plasmon resonance (SPR) experiments. hICOS-Fc, mICOS-Fc, and hCD28-Fc (all from R&D Systems) were affinity captured (350-400RU) by Protein A (Sigma Aldrich) previously amine coupled to a CM5 chip (GE Healthcare). hICOS-L.COMP was injected over each affinity captured protein at a concentration of 25 nM in HBS-EP running buffer (GE Healthcare).

ICOS-L.COMP Binding Kinetics

The kinetics of hICOSL-COMP, hICOSL-Fc, and COMP binding to immobilized hICOS-Fc was determined by SPR single cycle kinetic analysis using a Biacore T200. Briefly, titrated concentrations of ICOS-L.COMP, ICOSL-Fc (R&D Systems), or COMP (negative control) were injected over hICOS-Fc (R&D Systems) previously immobilized on a CM5 sensor chip (GE Healthcare). The derived sensorgrams were fit with a 1:1 binding model to determine on-rate (ka), off-rate (kd) and the dissociation constant (KD).

ICOS-L.COMP Binding to Human T-Cells

The ability of ICOSL-COMP to bind to human T-cells was demonstrated by flow cytometry. ICOS-L.COMP was derivatized with FITC following manufacturer's directions (Thermofisher). 1×105 CD3+ T-cells previously isolated from human donor PBMCs (STEMCELL technologies human CD3+ Isolation Kit) was incubated with 100 nM ICOS-L.COMP.FITC and PE-Cy7-anti-CD4 (Biolegend) for 20 minutes in FACS buffer (PBS+2% FBS+0.09% NaN3). Cells were subsequently washed and analysed by flow cytometry using a BD LSR cytometer. DAPI was used to exclude dead cells.

ICOS-L.COMP Competition Experiments (Human)

The ability of hICOS-L.COMP to compete with hICOS-Fc for binding to ICOS on 1-day anti-CD3/CD28 stimulated human CD3+ T-cells was evaluated by flow cytometry. Briefly, 200 nM ICOS-L.COMP.FITC or an equal volume of PBS (no ICOS-L.COMP) was pre-incubated with stimulated CD3+ T-cells for 15 minutes on ice prior to addition of 100 nM hICOS-L.Fc. Cells were washed and incubated with a PE labelled anti-human IgG-Fc secondary antibody (Biolegend), and analysed by flow cytometry using a BD LSR cytometer.

ICOS-L Competition Experiments (Mouse)

The ability of hICOS-L.COMP to compete with mICOS-Ig for binding to ICOS on primary murine CD4+ was evaluated by flow cytometry. Splenic murine CD4+ T-cells were isolated using the EasySep Mouse CD4+ T-cell isolation kit (STEMCELL Technologies) and activated for 48 hours by exposure to immobilized anti-CD3 antibody (clone 145-2C11, BioXcell) to upregulate ICOS expression. Activated T-cells were incubated with mICOS-Ig or mICOS-Ig combined with ICOS-L.COMP, and binding of mICOS-Ig detected using a PE conjugated anti-human IgG-Fc antibody (BioLegend).

MC38 Colon Carcinoma Mouse Model

The ability of ICOS-L.COMP to synergize with anti-PD-1 checkpoint blockade to restore anti-tumor immune responses and slow the progression of established tumors was demonstrated using the MC38 colon carcinoma model. Male C57BL/6 mice were injected with 2×105 MC38 tumor cells and tumors allowed to reach a size of 50-150 mm3 prior to treatment over the course of 7-10 days. Animals were subsequently injected with either PBS, anti-PD-1 (200 μg, clone RMPI-14), ICOS-L.COMP (100 μg) or anti-PD-1 combined with ICOS-L.COMP every 2-3 days for five injections. Tumor size was measured every other day using calipers and calculated using the formula: large diameter×small diameter 2×π/6. In some cases tumors were resected at day 10-12 post treatment initiation, enzymatically dissociated to a single cell suspension, and stained with anti-CD45, anti-CD4, anti-CD8, anti-FOXP3 and anti-ICOS to profile TIL populations after treatment.

### Example 4: ICOS-L.COMP Results

Rationale and Design of a Pentameric ICOS-L Fusion Protein

T-cells require two-signals to achieve activation in-vitro and in-vivo, with the first signal being delivered by the T-cell receptor (TCR) upon recognition of antigen displayed on the major histocompatible complex (MHC). Secondary co-stimulatory signals are delivered by a number of ligand:receptor interactions such as B7-½:CD28 to increase T-cell activity. ICOS, a member of the B7/CD28 family, is a co-stimulatory receptor which is upregulated by T-cells upon activation. Binding of ICOS by its ligand ICOS-L, which is expressed on antigen presenting cells (APC), leads to increased T-cell proliferation and cytokine production. Prior studies have demonstrated increased expression of ICOS on T-cells in cancer patients treated with anti-CTLA-4 (ipilimumab), with this upregulation being correlated with improved clinical outcome. Pre-clinical experiments have established a therapeutic utility in agonizing ICOS signalling to promote beneficial anti-tumor immune responses. Specifically, it was shown that agonizing ICOS using a whole-cell vaccine (ICOS-L expression on B16 melanoma tumor cells) in combination with anti-CTLA-4 could lead to a delay in tumor growth in the B16 melanoma model. Collectively these results confirm a role for an ICOS agonist as a synergistic therapeutic to established checkpoint blocking therapeutics (anti-PD-1 and anti-CTLA-4 monoclonal antibodies).

To this end, it was hypothesized that a soluble ICOS agonist could be derived by pentamerization of the ICOS-L extracellular binding domain. Due to an increased avidity and clustering, it was hypothesized that this pentameric version of the natural ligand for ICOS (ICOS-L) may agonize ICOS signalling to a greater extent than would agonistic ICOS monoclonal antibodies or a dimeric version of the ICOS-L ECD (i.e., ICOS-Fc).

A pentameric ICOS-L construct was created by genetic fusion of the ICOS-L ECD (IgV+IgC domain) to the COMP pentamerization domain (ICOS-L.COMP; see SEQ ID NO: 57 of Appendix 1 for sequence). ICOS-L.COMP was expressed in a mammalian cell expression system, yielding stable homopentamers of MW ˜300 kDa under non-reducing conditions (FIG. 8B).

ICOSL.COMP Binds to Human and Mouse ICOS but not CD28

hICOS-L.COMP was characterized for its binding to ICOS and the closely related family member CD28 by SPR. hICOSL-COMP at 25 nM readily bound both mouse and human ICOS as expected (FIG. 9A). Very modest to negligible binding to hCD28 was observed exemplifying the specificity of this interaction.

ICOS-L.COMP Binds to ICOS with Superior Affinity/Avidity than ICOSL-Fc

Surface plasmon resonance (SPR) assays were used to characterize the binding affinity of hICOS-L.COMP and ICOSL-Fc to hICOS. hICOS-L.COMP bound with an apparent KD of 0.9 nM, a value approximately 3-fold stronger then hICOSL-Fc (2.9 nM). COMP did not show binding to ICOS demonstrating that this interaction is due to specific ICOSL-ICOS binding (FIG. 9B).

hICOS-L.COMP Binds Directly to Human T-Cells and Competes with ICOSL-Fc

hICOSL-COMP was also characterized for its ability to bind to ICOS-expressing human CD3+ T-cells. For this, hICOSL-COMP was derivatized with FITC, yielding approximately 13-15 FITC/molecule. hICOSL.COMP.FITC was found to readily bind CD3+CD4+ and CD3+CD4− T-cells isolated from human PBMCs (FIG. 9C). Additionally, the binding of ICOS-L.COMP completely inhibited binding of ICOSL-Fc to stimulated human T-cells, confirming that ICOS-L.COMP and ICOSL-Fc compete for binding to cellular expressed ICOS (FIG. 9D).

ICOS-L Competition Experiments (Mouse)

The ability of hICOS-L.COMP to outcompete mICOS-Ig for binding to ICOS displayed on primary murine CD4+ was evaluated by flow cytometry. The pentameric hICOS-L.COMP sufficiently outcompetes mICOS-L-Ig for binding to ICOS expressed by activated T-cells (FIG. 9E).

ICOS-L.COMP Co-Stimulates Human T-Cells

The function of ICOS-L.COMP in agonizing ICOS signalling to co-stimulate human T-cells was established. CFSE labelled CD3+ T-cells isolated from fresh human cord blood were stimulated in vitro with anti-CD3 antibody, and ICOS-L.COMP or COMP (negative control) added soluble to the culture media. Cell expansion and proliferation was tracked after 72 hours by FACS, demonstrating that ICOS-L.COMP, but not COMP, profoundly stimulates the proliferation of CD4+ and CD8+ T-cells (FIG. 10A). Additionally, ICOS-L.COMP, induced upregulation of the T-cell activation marker CD25 (FIG. 10B). In stark contrast to hICOSL-COMP, ICOSL-Fc as a soluble ligand was not capable of inducing the expansion and proliferation of CD3+CD4+ T-cells (FIG. 10C). Lastly, ICOSL-COMP did not stimulate T-cells in the absence of anti-CD3 induced T-cell receptor signalling confirming the function of ICOSL-COMP as a co-stimulatory ligand (FIG. 10D). Similar results were also seen in the co-stimulation of T-cells isolated from adult human PBMCs (FIG. 10E). Furthermore, soluble ICOSL-COMP co-stimulation, but not ICOSL-Fc, led to substantial increases in the secretion of Th1 cytokines IFNγ, TNFα, and IL10, and modest but significant increases in IL2 and IL6. (FIG. 10F). Collectively these experiments demonstrate that soluble ICOS-L.COMP readily agonizes the ICOS signalling pathway to stimulate human T-cell proliferation and activation.

ICOS-L.COMP Synergizes with Checkpoint Blockade to Promote Protective Anti-Tumor Immunity in Mice

To determine the anti-tumor efficacy of ICOS-L.COMP in combination with checkpoint blockade, the MC38 colon carcinoma model was used. Here, immune competent C57BI/6 mice with injected subcutaneously with MC38 cells and tumors established to a volume of 50-150 mm3 prior to treatment with PBS (control), anti-PD-1 monotherapy, ICOS-L.COMP monotherapy, or ICOS-L.COMP combined with anti-PD-1 (combination) (FIG. 11A). Combination treatment with ICOS-L.COMP and anti-PD-1 led to a significantly delayed tumor growth relative to anti-PD-1 monotherapy (**P<0.01) (FIG. 11B). Individual tracing of each animal's tumors shows complete responses were observed in 3/12 animals in the combination group at the time of last treatment (FIG. 11C), with zero instances of complete regression in any other group. Animals in the ICOS-L.COMP monotherapy group did not exhibit improved outcome compared to PBS controls suggesting the need for checkpoint blockade combination in this model (FIG. 12A). Supporting a role for the combination approach, tumor infiltrating CD4+ and CD8+ T-cells had significantly higher ICOS expression and an overall increase in ICOS+ cells within the tumor (FIG. 12B). Together this suggests that the efficacy of checkpoint blocking antibodies which upregulate ICOS expression and increase the abundance of TILs can be potentiated by a combination treatment with a soluble ICOS agonist. Supportingly, combined treatment with anti-PD-1 and ICOS-L.COMP led to modest but significant increases in CD4+FOXP3− (CD4 effector) cells within the TIL compartment (CD45+) compared to anti-PD-1 monotherapy alone (*P<0.05) (FIG. 12C). There were no changes in the abundance of Treg cells (CD4+FOXP3+) (FIG. 12D).

Collectively the data show that an ICOS-L ECD pentamer (ICOS-L.COMP) can readily bind to ICOS with high-avidity to co-stimulate human T-cells. Importantly, this co-stimulation in-vivo can lead to an increase in anti-tumor activity when combined with checkpoint blockade (anti-PD-1 antibody) leading to a reduced tumor burden.

### Example 5: PD-L1.COMP Methods

Design of a PD-L1 Pentamer (PD-L1.COMP) Expression Vector

A codon optimized dsDNA construct was synthesized (IDT) encoding the mouse PD-L1 ECD bearing EcoRI (5′) and KpnI (3′) restriction sites. This construct was digested, and ligated into a EcoRI/KpnI double digested plasmid (COMP.HIS8-pcDNA3.4) such that the PD-L1 ECD (SEQ ID NO:42) was located downstream an Ig-kappa leader sequence and upstream the COMP pentamerization domain (SEQ ID NO: 12) (PD-L1.COMP; see SEQ ID NO: 46 of Appendix 1 for sequence).

Expression and Purification of Chimeric PD-L1.COMP

The PD-L1.COMP encoding plasmid was transfected into Expi293F cells following manufacturers recommendations (GeneArt; Thermo Fisher Scientific) and a stable cell line secreting PD-L1.COMP selected by exposing the transfected cell to geneticin (GeneArt; Thermo Fisher Scientific) for two weeks. Secreted histidine-tagged PD-L1.COMP was purified from cell-culture supernatants using HisTrap HP columns (GE Healthcare). Subsequent to purification, protein samples were desalted into PBS, pH 7.4, using PD10 columns (GE Healthcare). Proteins were verified for purity using SDS-PAGE, and protein concentration quantified by BCA assay (Pierce) or A280 measurements.

T-Cell Line Activation and Proliferation Assays

The murine 2.10 T-cell clone was cultured in complete IMDM supplemented with IL-2 (3.5 μg/mL), lecithin (20 μg/mL), and BSA (0.5 mg/mL). For activation assays, cells were harvested, washed three times and seeded on an anti-CD3 (3 ug/mL, BioXcell) coated 96-well plate at 2×104 cells/well. PD-L1.COMP and COMP (negative control) were either coated in the anti-CD3 coated wells in PBS pH7.4 for 1 hour at 37° C. or added to the wells soluble with the 2.10 cells, at 10 ug/mL construct concentration. Cells were cultured for 18 hours then pulsed with 1 μCi [3H]-thymidine and cultured for another 6 hours. [3H]-thymidine uptake and proliferation quantified using TopCount NXT scintillation counter (Perkin Elmer).

Binding to a PD-1 Expression T-Cell Clone

The binding of PD-L1.COMP to cell expressed PD-1 was investigating by flow cytometry using the 2.10 T-cell line. PD-L1.COMP was biotinylated using the EX-Link Sulfo-NHS-LC-Biotin reagent following manufacturer's directions (Thermo Scientific) and desalted into PBS using a PD10 column to remove excess biotin reagent. Resting or anti-CD3 activated 2.10 cells were incubated with biotinylated COMP or PD-L1.COMP for 30 minutes at 4 C. Cells were subsequently washed in PBS and stained with Streptavidin-PE (1:100, BioLegend) for 30 minutes at 4 C. The cells were washed with PBS, resuspended in PBS+DAPI (for live/dead cell exclusion), and read using a FACScalibur cell analyzer (Becton Dickinson).

Primary CD4+ T-Cell Line Activation and Proliferation

CD4+ T-cells were isolated from murine splenocytes using the mouse CD4+ T-cell isolation kit (STEM CELL Technologies). CD4+ T cells were CFSE labeled using manufacturer protocol (Thermo Fischer Scientific), and seeded on an anti-CD3 (3 ug/mL) coated 96-well plate, with PD-L1.COMP, hB7-H4.COMP, mVISTA.COMP or COMP added soluble in culture media at 10 μg/mL. Cells were harvested after 72 hours and CFSE profiles analysed by FACS (FACSCalibur, Becton Dickinson). In some cases, culture media from the CFSE labeled cells were collected at 48 and 72 hour time points, and IL-2 and IFNγ secretion quantified by ELISA (R&D Systems).

### Example 6: PD-L1.COMP Results

Stimulation of negative checkpoint receptors occurs through the binding of an IgV domain displayed by a protein ligand, such as PD-L1, expressed on APCs and tumour cells to a complementary IgV domain of its cognate immune checkpoint receptor, such as PD-1, on T-cells. Past studies have demonstrated that monomeric forms of these IgV domains involving PD1:PD-L1 and CD28:CD80/CD86 interact with each other with modest affinity, reflected by Kd values typically in the low micromolar (μM) range (References 40, 41). To activate checkpoint receptors on T-cells in-vitro, these immune checkpoint ligands have been expressed as dimers, such as Fc fusion proteins, which have been immobilized on a surface. The immobilized presentation mimics avidity events taking place when such immune checkpoint domains are displayed on the surface of APCs and T-cells. Previous reports have shown that PD-L1-Fc requires immobilization on plates or beads to successfully agonist PD-1 immunoinhibitory signalling suggesting that use of PD-L1-Fc in-vivo to suppress T-cell activity may be limited due to its inability to fully agonize PD-1.

Without being bound by theory, the lack of activity in soluble PD-L1 in-vitro may be caused by insufficient avidity towards its receptor and/or a lack of ability to cluster PD-1. This hypothesis is further supported by the finding that a soluble VISTA IgV pentamer (VISTA.COMP) could readily suppress T-cell proliferation in-vitro while the dimeric VISTA-Fc could not.

Design, Expression, and Purification of a PD-L1 Homopentamer: PD-L1.COMP

A higher order PD-L1 ECD multimer was engineered in order to generate a PD-1 agonist that may effectively suppress T-cell stimulation both in-vitro and in-vivo. A recombinant PD-L1 pentamer (PD-L1.COMP; see SEQ ID No: 46 in Appendix 1 for sequence) was constructed by genetically fusing the mouse PD-L1 ECD domain to the COMP pentamerization domain. Recombinant PD-L1.COMP was produced in a mammalian expression system, yielding a pentameric protein of ˜250-300 kDa stabilized by intramolecular disulphide bonds within the COMP pentamerization domain (FIG. 5A).

PD-L1.COMP Binds to PD-1 Expressed by a T-Cell Line

Flow cytometry was performed on the 2.10 T-cell line using biotinylated PD-L1.COMP or COMP to establish that PD-L1.COMP binds to PD-1 expressed in a cell context. PD-L1.COMP and COMP were labelled with an equivalent number of biotin groups and used to stain naïve or anti-CD3 activated 2.10 cells. PD-L1.COMP, but not COMP readily bound to naïve 2.10 T-cells, with the amount of binding increased throughout T-cell activation, consistent with the established kinetics of PD-1 upregulation during T-cell activation (FIG. 13).

Primary CD4+ T-Cell Line Activation and Proliferation

In contrast to PD-L1-Fc which requires immobilization to agonize PD-1 and suppress T-cell activity, soluble PD-L1.COMP completely suppressed the expansion and proliferation of CFSE labelled primary murine CD4+ T-cells undergoing anti-CD3 antibody mediated stimulation. Importantly, recombinant COMP domain alone did not significantly inhibit T-cell proliferation confirming that the immunosuppressive effects seen with PD-L1.COMP are not off-target events due to the COMP pentamerization domain or histidine tag (FIG. 6A).

### Example 7: B7-H4.COMP Methods

Design of a Pentameric B7-H4 Construct

The codon optimized gene fragment encoding human B7-H4 fused to the COMP pentamerization domain was synthesized (GeneArt, Thermo Fisher Scientific) and cloned into the pcDNA3.4 expression plasmid with a 5′ Ig-Kappa leader sequence. The final construct consists of dsDNA encoding the human B7-H4 ECD (SEQ ID NO: 25) followed by a spacer sequence, the COMP pentamerization domain (SEQ ID NO: 11) followed by another spacer sequence and a HIS8 tag (B7-H4.COMP; see SEQ ID NO: 30 of Appendix 1 for sequence).

Expression and Purification of Pentameric B7-H4.COMP

hB7-H4.COMP was expressed using the Expi293 transient mammalian expression system using manufacturer's protocols (Thermo Fisher Scientific). Secreted hB7-H4.COMP was dialysed against PBS and purified by Ni-NTA purification using HisTrap HP columns. The protein was desalted into PBS pH 7.4 using a PD-10 column (GE Healthcare) and purity verified by SDS-PAGE and concentration determined using A280 measurements.

2.10 T-Cell Activation Assay

The clonal IL-2 dependent 2.10 T-cell was cultured in complete IMDM supplemented with IL-2 (3.5 μg/mL), lecithin (20 μg/mL), and BSA (0.5 mg/mL). Cells were harvested, washed three times, resuspended in complete IMDM without IL-2 and lecithin and seeded on an anti-CD3 (3 ug/mL, BioXcell) coated 96-well plate at 2×104 cells/well. hB7-H4.COMP, Vista.COMP (positive control) and COMP (negative control) were either coated in the anti-CD3 coated wells in PBS for 1 hour at 37° C. and well subsequently washed, or added to directly to the wells soluble with the 2.10 cells. Cells were cultured for 18 hours then pulsed with 1 uCi [3H]-thymidine and cultured for another 6 hours. [3H]-thymidine uptake and proliferation was quantified using TopCount NXT scintillation counter (Perkin Elmer).

Primary CD4+ T-Cell Line Activation and Proliferation

CD4+ T-cells were isolated from murine splenocytes using the mouse CD4+ T-cell isolation kit (STEM CELL Technologies). CD4+ T cells were CFSE labeled using manufacturer protocol (Thermo Fischer Scientific), and seeded on an anti-CD3 (3 ug/mL) coated 96-well plate, with PD-L1.COMP, hB7-H4.COMP, mVISTA.COMP or COMP added soluble in culture media at 10 μg/mL. Cells were harvested after 72 hours and CFSE profiles analysed by FACS (FACSCalibur, Becton Dickinson). In some cases, culture media from the CFSE labeled cells were collected at 48 and 72 hour time points, and IL-2 and IFNγ secretion quantified by ELISA (R&D Systems).

Binding to a T-Cell Clone

The binding of B7-H4.COMP to its putative receptor (B7-H4R) expressed on T-cells was investigating by flow cytometry using the 2.10 T-cell line. B7-H4.COMP was biotinylated using the EX-Link Sulfo-NHS-LC-Biotin reagent following manufacturer's directions (Thermo Scientific) and desalted into PBS using a PD10 column to remove excess biotin reagent. Resting or anti-CD3 activated 2.10 cells were incubated with biotinylated COMP or B7-H4.COMP for 30 minutes at 4 C. Cells were subsequently washed in PBS and stained with Streptavidin-PE (1:100, BioLegend) for 30 minutes at 4 C. The cells were washed with PBS, resuspended in PBS+DAPI (for live/dead cell exclusion), and read using a FACScalibur cell analyzer (Becton Dickinson).

### Example 8: B7-H4.COMP Results

Previous reports have shown that B7-H4-Fc requires immobilization on plates or beads to successfully agonise immunoinhibitory signalling in T-cells suggesting that use of B7-H4-Fc in-vivo to suppress T-cell activity may be limited due to its inability to fully agonize the putative receptor in the absence of FcR-mediated cross-linking.

Without being bound by theory, the lack of activity of soluble B7-H4-Fc in-vitro may be caused by insufficient avidity towards its receptor and/or a lack of ability to cluster the putative receptor. This hypothesis is further supported by the finding that a soluble VISTA IgV pentamer (VISTA.COMP) could readily suppress T-cell proliferation in-vitro while the dimeric VISTA-Fc could not.

Design and Expression of a B7-H4 Pentamer

To address the issue of insufficient avidity or lack of clustering, a pentameric version of human B7-H4 was generated, by fusing the B7-H4 ECD with the COMP pentamerization domain (B7-H4.COMP; see SEQ ID NO: 30 of Appendix 1 for sequence). B7-H4.COMP was readily produced in mammalian cells and purified to homogeneity as stable pentamers at the MW of 250-300 kDa (FIG. 5A).

hB7-H4.COMP Binds to a Clonal T-Cell Line and Suppresses its Activation

Flow cytometry was performed on the 2.10 T-cell line using biotinylated B7-H4.COMP or COMP to establish that B7-H4.COMP binds to the putative B7-H4R expressed on T-cells. B7-H4.COMP and COMP were labelled with an equivalent number of biotin groups and used to stain naïve or anti-CD3 activated 2.10 cells. B7-H4.COMP, but not COMP readily bound to naïve and activated 2.10 T-cells (FIG. 13).

The IL-2 dependent 2.10 T-cell line was used as a reporter system to assay if B7-H4.COMP could suppress T-cell activation. Consistent with previous observations using pentameric VISTA.COMP, B7-H4.COMP suppressed anti-CD3 induced proliferation whether present as an immobilized ligand or provided in a soluble form in culture media (p<0.01) (FIG. 14). The COMP pentamerization domain did not substantially alter proliferation when added as a soluble ligand, confirming on-target effects of VISTA.COMP and B7-H4.COMP.

B7-H4.COMP Inhibits Primary CD4+ T-Cell Line Activation and Proliferation

In contrast to B7-H4-Fc which requires immobilization to agonize B7-H4R and suppress T-cell activity, soluble B7-H4.COMP completely suppressed the expansion and proliferation of CFSE labelled primary murine CD4+ T-cells undergoing anti-CD3 antibody mediated stimulation (FIG. 15). Importantly, recombinant COMP domain alone did not significantly inhibit T-cell proliferation confirming the that the immunosuppressive effects seen with B7-H4.COMP are not off-target events due to the COMP pentamerization domain or histidine tag. In addition, soluble B7-H4.COMP significantly diminished (p<0.01) the secretion of the inflammatory cytokine, IL-2, by stimulated CD4+ T-cells (FIG. 16).

Pentamerization of Checkpoint Ligands as a Strategy to Design High-Avidity Checkpoint Receptor Agonists

The data provided herein indicate that pentamerization of three checkpoint ligands, namely PD-L1, B7-H4, and VISTA, can be used to design high-avidity checkpoint receptor agonist capable of suppressing T-cell activities in-vitro and capable of suppressing inflammatory responses in-vivo. Comparisons between immobilized and soluble VISTA-Fc and VISTA.COMP show that activity as checkpoint-receptor agonists is dependent on the level of oligomerization, with the higher-avidity multimer created using the COMP pentamerization domain being required for activity in solution (i.e., in the absence of immobilization to a substrate). The inventors have found the COMP domain to be a useful scaffold for expressing stable pentamers upon fusion of the ECD from checkpoint ligands. The data provided herein, combined with the observation of exacerbated autoimmune diseases observed upon genetic deletion of checkpoint ligands and receptors in mice, suggest a potential utility in agonizing these checkpoint receptors with pentameric agonists to clinically suppress undesired immune responses.

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference as if set forth in their entirety.

APPENDIX 1: SEQUENCES

VISTA extracellular domain cDNA sequence (human) (SEQ ID NO: 1)
ttc
aaggtcgcca cgccgtattc cctgtatgtc tgtcccgagg ggcagaacgt caccctcacc
tgcaggctct tgggccctgt ggacaaaggg cacgatgtga ccttctacaa gacgtggtac
cgcagctcga ggggcgaggt gcagacctgc tcagagcgcc ggcccatccg caacctcacg
ttccaggacc ttcacctgca ccatggaggc caccaggctg ccaacaccag ccacgacctg
gctcagcgcc acgggctgga gtcggcctcc gaccaccatg gcaacttctc catcaccatg
cgcaacctga ccctgctgga tagcggcctc tactgctgcc tggtggtgga gatcaggcac
caccactcgg agcacagggt ccatggtgcc atggagctgc aggtgcagac aggcaaagat
gcaccatcca actgtgtggt gtacccatcc tcctcccagg atagtgaaaa catcacggct
VISTA extracellular domain cDNA sequence (mouse) (SEQ ID NO: 2)
ttcaaggtca ccactccata
ttctctctat gtgtgtcccg agggacagaa tgccaccctc acctgcagga ttctgggccc
cgtgtccaaa gggcacgatg tgaccatcta caagacgtgg tacctcagct cacgaggcga
ggtccagatg tgcaaagaac accggcccat acgcaacttc acattgcagc accttcagca
ccacggaagc cacctgaaag ccaacgccag ccatgaccag ccccagaagc atgggctaga
gctagcttct gaccaccacg gtaacttctc tatcaccctg cgcaatgtga ccccaaggga
cagcggcctc tactgctgtc tagtgataga attaaaaaac caccacccag aacaacggtt
ctacgggtcc atggagctac aggtacaggc aggcaaaggc tcggggtcca catgcatggc
gtctaatgag caggacagtg acagcatcac ggct
COMP pentamerization domain cDNA sequence (human) (SEQ ID NO: 3)
gacctgggcc cgcagatgct tcgggaactg caggaaacca acgcggcgct gcaggacgtg
cgggagctgc tgcggcagca ggtcagggag atcacgttcc tgaaaaacac ggtgatggag
tgtgacgcgt gcggg
COMP pentamerization domain cDNA sequence (mouse) (SEQ ID NO: 4)
gacct ggccccacag
atgctgcgag aacttcagga gactaatgcg gcgctgcaag acgtgagaga gctgttgcga
cagcaggtca aggagatcac cttcctgaag aatacggtga tggaatgtga tgcttgcgga
VISTA extracellular domain mRNA sequence (human) (SEQ ID NO: 5)
uuc
aaggucgcca cgccguauuc ccuguauguc ugucccgagg ggcagaacgu cacccucacc
ugcaggcucu ugggcccugu ggacaaaggg cacgauguga ccuucuacaa gacgugguac
cgcagcucga ggggcgaggu gcagaccugc ucagagcgcc ggcccauccg caaccucacg
uuccaggacc uucaccugca ccauggaggc caccaggcug ccaacaccag ccacgaccug
gcucagcgcc acgggcugga gucggccucc gaccaccaug gcaacuucuc caucaccaug
cgcaaccuga cccugcugga uagcggccuc uacugcugcc ugguggugga gaucaggcac
caccacucgg agcacagggu ccauggugcc auggagcugc aggugcagac aggcaaagau
gcaccaucca acuguguggu guacccaucc uccucccagg auagugaaaa caucacggcu
VISTA extracellular domain mRNA sequence (mouse) (SEQ ID NO: 6)
uucaagguca ccacuccaua
uucucucuau gugugucccg agggacagaa ugccacccuc accugcagga uucugggccc
cguguccaaa gggcacgaug ugaccaucua caagacgugg uaccucagcu cacgaggcga
gguccagaug ugcaaagaac accggcccau acgcaacuuc acauugcagc accuucagca
ccacggaagc caccugaaag ccaacgccag ccaugaccag ccccagaagc augggcuaga
gcuagcuucu gaccaccacg guaacuucuc uaucacccug cgcaauguga ccccaaggga
cagcggccuc uacugcuguc uagugauaga auuaaaaaac caccacccag aacaacgguu
cuacgggucc auggagcuac agguacaggc aggcaaaggc ucggggucca caugcauggc
gucuaaugag caggacagug acagcaucac ggcu
COMP pentamerization domain mRNA sequence (human) (SEQ ID NO: 7)
gaccugggcc cgcagaugcu ucgggaacug caggaaacca acgcggcgcu gcaggacgug
cgggagcugc ugcggcagca ggucagggag aucacguucc ugaaaaacac ggugauggag
ugugacgcgu gcggg
COMP pentamerization domain mRNA sequence (mouse) (SEQ ID NO: 8)
gaccu ggccccacag
augcugcgag aacuucagga gacuaaugcg gcgcugcaag acgugagaga gcuguugcga
cagcagguca aggagaucac cuuccugaag aauacgguga uggaauguga ugcuugcgga
VISTA extracellular domain amino acid sequence (human) (SEQ ID NO: 9)
fkvatpys lyvcpegqnv tltcrllgpv
dkghdvtfyk twyrssrgev qtcserrpir nltfqdlhlh hgghqaants hdlaqrhgle
sasdhhgnfs itmrnltlld sglycclvve irhhhsehrv hgamelqvqt gkdapsncvv
ypsssqdsen ita
VISTA extracellular domain amino acid sequence (mouse) (SEQ ID NO: 10)
fkvttpys lyvcpegqna tltcrilgpv
skghdvtiyk twylssrgev qmckehrpir nftlqhlqhh gshlkanash dqpqkhglel
asdhhgnfsi tlrnvtprds glycclviel knhhpeqrfy gsmelqvqag kgsgstcmas
neqdsdsita
COMP pentamerization domain amino acid sequence (human) (SEQ ID NO: 11)
dl gpqmlrelqe tnaalqdvre llrqqvreit flkntvmecd acg
COMP pentamerization domain amino acid sequence (mouse) (SEQ ID NO: 12)
dla pqmlrelqet naalqdvrel lrqqvkeitf lkntvmecda cg
Codon Optimized mVISTA.COMP DNA sequence (mouse) (SEQ ID NO: 13)

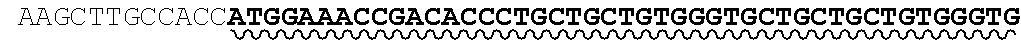

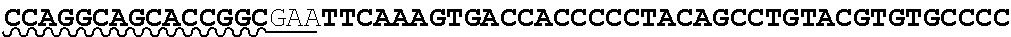

GAGGGCCAGAATGCCACCCTGACCTGTAGAATCCTGGGCCCCGTGTCCAAGGGCCA
CGACGTGACCATCTACAAGACCTGGTATCTGAGCAGCAGAGGCGAGGTGCAGATGT
GCAAAGAGCACCGGCCCATCCGGAACTTCACCCTGCAGCATCTGCAGCACCACGGC
AGCCACCTGAAGGCCAATGCCAGCCACGACCAGCCTCAGAAGCACGGCCTGGAACT
GGCCTCTGACCACCACGGAAACTTCAGCATCACCCTGCGGAACGTGACCCCCAGAG
ACAGCGGCCTGTACTGCTGTCTCGTGATCGAGCTGAAGAACCACCACCCCGAGCAGC
GGTTCTACGGCAGCATGGAACTGCAGGTCCAGGCCGGCAAGGGCAGCGGCTCTACT
TGCATGGCCAGCAACGAGCAGGACAGCGACTCCATCACAGCCGAATTCGGCTCTGG

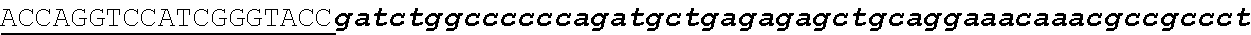

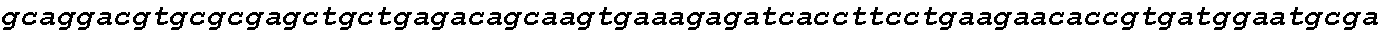

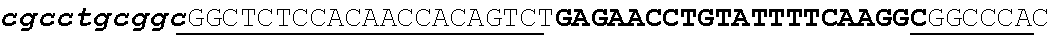

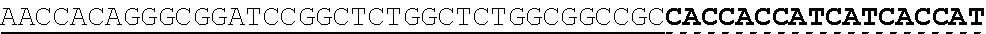

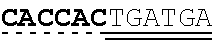

non-bold = nucleotides that are not translated
wavy underline = DNA sequencing encoding IgKappa secretion signal
uppercase, no underline (black) = VISTA extracellular domain
solid underlined (gray) = Spacer sequences
lower case italicized = COMP pentamerization domain
stippled underline = DNA sequence encoding a his-tag
double underlined = stop codon
bold = nucleotides that are translated
mVISTA.COMP amino acid sequence (mouse) (SEQ ID NO: 14)

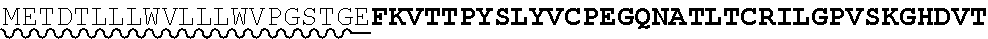

IYKTWYLSSRGEVQMCKEHRPIRNFTLQHLQHHGSHLKANASHDQPQKHGLELASDHHG
N
FSITLRNVTPRDSGLYCCLVIELKNHHPEQRFYGSMELQVQAGKGSGSTCMASNEQDSDS
ITAEFGSGPGPSGTdlapqmlrelqetnaalqdvrellrqqvkeitflkntvmecdacgG

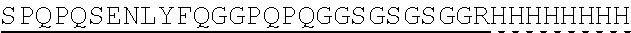

wavy underline = IgKappa secretion signal
Bold (black) = VISTA extracellular domain containing IgV domain
lower case italicized = COMP pentamerization domain
stippled underline = Histidine tag
underlined (gray) = Spacer sequences
Full length VISTA amino acid sequence (human) (SEQ ID NO: 15)
ACCESSION Q9H7M9

| | |
|---|---|
| 1 | mgvptaleag swrwgsllfa lflaaslgpv aafkvatpys lyvcpegqnv tltcrllgpv |
| 61 | dkghdvtfyk twyrssrgev qtcserrpir nltfqdlhlh hgghqaants hdlaqrhgle |
| 121 | sasdhhgnfs itmrnltlld sglycclvve irhhhsehry hgamelqvqt gkdapsncvv |
| 181 | ypsssqdsen itaaalatga civgilclpl illlvykqrq aasnrraqel vrmdsniqgi |
| 241 | enpgfeaspp aqgipeakvr hplsyvaqrq psesgrhlls epstplsppg pgdvffpsld |
| 301 | pvpdspnfev i |

Bold = VISTA extracellular domain containing IgV domain (amino acids 31-193)
Full length VISTA amino acid sequence (mouse) (SEQ ID NO: 16)
ACCESSION Q9D659

| | |
|---|---|
| 1 | mgvpavpeas sprwgtllla iflaasrglv aafkvttpys lyvcpegqna tltcrilgpv |
| 61 | skghdvtiyk twylssrgev qmckehrpir nftlqhlqhh gshlkanash dqpqkhglel |
| 121 | asdhhgnfsi tlrnvtprds glycclviel knhhpeqrfy gsmelqvqag kgsgstcmas |
| 181 | neqdsdsita aalatgaciv gilclplill lvykqrqvas hrraqelvrm dsntqgienp |
| 241 | gfettppfqg mpeaktrppl syvaqrqpse sgryllsdps tplsppgpgd vffpsldpvp |
| 301 | dspnseai |

Bold = VISTA extracellular domain containing IgV domain (amino acids 31-193)
Full length COMP amino acid sequence (human) (SEQ ID NO: 17)
ACCESSION NP_000086

| | |
|---|---|
| 1 | mvpdtacvll ltlaalgasg qgqsplgsdl gpqmlrelge tnaalqdvre llrqqvreit |
| 61 | flkntvmecd acgmqqsvrt glpsvrpllh capgfcfpgv aciqtesgar cgpcpagftg |
| 121 | ngshctdvne cnahpcfprv rcintspgfr ceacppgysg pthqgvglaf akankqvctd |
| 181 | inecetgqhn cvpnsvcint rgsfqcgpcq pgfvgdqasg cqrraqrfcp dgspsecheh |
| 241 | adcvlerdgs rscvcavgwa gngilcgrdt dldgfpdekl rcperqcrkd ncvtvpnsgq |
| 301 | edvdrdgigd acdpdadgdg vpnekdncpl vrnpdqrntd edkwgdacdn crsqknddqk |
| 361 | dtdqdgrgda cdddidgdri rnqadncpry pnsdqkdsdg dgigdacdnc pqksnpdqad |
| 421 | vdhdfvgdac dsdqdqdgdg hqdsrdncpt vpnsaqedsd hdgqgdacdd dddndgvpds |
| 481 | rdncrlvpnp gqedadrdgv gdvcqddfda dkvvdkidvc penaevtltd frafqtvvld |
| 541 | pegdaqidpn wvvinqgrei vqtmnsdpgl avgytafngv dfegtfhvnt vtdddyagfi |
| 601 | fgyqdsssfy vvmwkqmeqt ywqanpfrav aepgiqlkav ksstgpgeql rnalwhtgdt |
| 661 | esqvrllwkd prnvgwkdkk syrwflqhrp qvgyirvrfy egpelvadsn vvldttmrgg |
| 721 | rlgvfcfsqe niiwanlryr cndtipedye thqlrqa |

Bold = Pentamerization domain (amino acids 28-73)
Full length COMP amino acid sequence (mouse) (SEQ ID NO: 18)
ACCESSION NP_057894

| | |
|---|---|
| 1 | mgptacvlvl alailratgq gqiplggdla pqmlrelget naalqdvrel lrqqvkeitf |
| 61 | lkntvmecda cgmqpartpg lsvrpvplca pgscfpgvvc setatgarcg pcppgytgng |
| 121 | shctdvnecn ahpcfprvrc intspgfhce acppgfsgpt hegvgltfak snkqvctdin |
| 181 | ecetgqhncv pnsvcvntrg sfqcgpcqpg fvgdqtsgcq rrgqhfcpdg spspchekan |
| 241 | cvlerdgsrs cvcavgwagn gllcgrdtdldgfpdeklrc serqcrkdnc vtvpnsgqed |
| 301 | vdrdgigdac dpdadgdgvp neqdncplvr npdqrnsdsd kwgdacdncr skknddqkdt |
| 361 | dldgrgdacd ddidgdrirn vadncprvpn fdqsdsdgdg vgdacdncpq kdnpdqrdvd |
| 421 | hdfvgdacds dqdqdgdghq dsrdncptvp nsaqqdsdhd gkgdacdddd dndgvpdsrd |
| 481 | ncrlvpnpgq edndrdgvgd acqgdfdadk vidkidvcpe naevtltdfr afqtvvldpe |
| 541 | gdaqidpnwv vinqgmeivq tmnsdpglav gytafngvdf egtfhvntat dddyagfifg |
| 601 | yqdsssfyvv mwkqmeqtyw qanpfravae pgiqlkavks stgpgeqlrn alwhtgdtas |
| 661 | qvrllwkdpr nvgwkdktsy rwflqhrpqv gyirvrfyeg pelvadsnw ldtamrggrl |
| 721 | gvfcfsqeni iwanlryrcn dtipedyesh rlqry |

Bold = Pentamerization domain (amino acids 28-72)
VISTA cDNA nucleotide sequence (human) (SEQ ID NO: 19)
ACCESSION NM_022153

| | |
|---|---|
| 1 | gggggcgggt gcctggagca cggcgctggg gccgcccgca gcgctcactc gctcgcactc |
| 61 | agtcgcggga ggcttccccg cgccggccgc gtcccgcccg ctccccggca ccagaagttc |
| 121 | ctctgcgcgt ccgacggcga catgggcgtc cccacggccc tggaggccgg cagctggcgc |
| 181 | tggggatccc tgctcttcgc tctcttcctg gctgcgtccc taggtccggt ggcagccttc |
| 241 | aaggtcgcca cgccgtattc cctgtatgtc tgtcccgagg ggcagaacgt caccctcacc |
| 301 | tgcaggctct tgggccctgt ggacaaaggg cacgatgtga ccttctacaa gacgtggtac |
| 361 | cgcagctcga ggggcgaggt gcagacctgc tcagagcgcc ggcccatccg caacctcacg |
| 421 | ttccaggacc ttcacctgca ccatggaggc caccaggctg ccaacaccag ccacgacctg |
| 481 | gctcagcgcc acgggctgga gtcggcctcc gaccaccatg gcaacttctc catcaccatg |
| 541 | cgcaacctga ccctgctgga tagcggcctc tactgctgcc tggtggtgga gatcaggcac |
| 601 | caccactcgg agcacagggt ccatggtgcc atggagctgc aggtgcagac aggcaaagat |
| 661 | gcaccatcca actgtgtggt gtacccatcc tcctcccagg atagtgaaaa catcacggct |
| 721 | gcagccctgg ctacgggtgc ctgcatcgta ggaatcctct gcctccccct catcctgctc |
| 781 | ctggtctaca agcaaaggca ggcagcctcc aaccgccgtg cccaggagct ggtgcggatg |
| 841 | gacagcaaca ttcaagggat tgaaaacccc ggctttgaag cctcaccacc tgcccagggg |
| 901 | atacccgagg ccaaagtcag gcaccccctg tcctatgtgg cccagcggca gccttctgag |
| 961 | tctgggcggc atctgctttc ggagcccagc acccccctgt ctcctccagg ccccggagac |
| 1021 | gtcttcttcc catccctgga ccctgtccct gactctccaa actttgaggt catctagccc |
| 1081 | agctggggga cagtgggctg ttgtggctgg gtctggggca ggtgcatttg agccagggct |
| 1141 | ggctctgtga gtggcctcct tggcctcggc cctggttccc tccctcctgc tctgggctca |
| 1201 | gatactgtga catcccagaa gcccagcccc tcaacccctc tggatgctac atggggatgc |
| 1261 | tggacggctc agcccctgtt ccaaggattt tggggtgctg agattctccc ctagagacct |
| 1321 | gaaattcacc agctacagat gccaaatgac ttacatctta agaagtctca gaacgtccag |
| 1381 | cccttcagca gctctcgttc tgagacatga gccttgggat gtggcagcat cagtgggaca |
| 1441 | agatggacac tgggccaccc tcccaggcac cagacacagg gcacggtgga gagacttctc |
| 1501 | ccccgtggcc gccttggctc ccccgttttg cccgaggctg ctcttctgtc agacttcctc |
| 1561 | tttgtaccac agtggctctg gggccaggcc tgcctgccca ctggccatcg ccaccttccc |
| 1621 | cagctgcctc ctaccagcag tttctctgaa gatctgtcaa caggttaagt caatctgggg |
| 1681 | cttccactgc ctgcattcca gtccccagag cttggtggtc ccgaaacggg aagtacatat |
| 1741 | tggggcatgg tggcctccgt gagcaaatgg tgtcttgggc aatctgaggc caggacagat |
| 1801 | gttgccccac ccactggaga tggtgctgag ggaggtgggt ggggccttct gggaaggtga |
| 1861 | gtggagaggg gcacctgccc cccgccctcc ccatccccta ctcccactgc tcagcgcggg |
| 1921 | ccattgcaag ggtgccacac aatgtcttgt ccaccctggg acacttctga gtatgaagcg |
| 1981 | ggatgctatt aaaaactaca tggggaaaca ggtgcaaacc ctggagatgg attgtaagag |
| 2041 | ccagtttaaa tctgcactct gctgctcctc ccccaccccc accttccact ccatacaatc |
| 2101 | tgggcctggt ggagtcttcg cttcagagcc attcggccag gtgcgggtga tgttcccatc |
| 2161 | tcctgcttgt gggcatgccc tggctttgtt tttatacaca taggcaaggt gagtcctctg |
| 2221 | tggaattgtg attgaaggat tttaaagcag gggaggagag tagggggcat ctctgtacac |
| 2281 | tctgggggta aaacagggaa ggcagtgcct gagcatgggg acaggtgagg tggggctggg |
| 2341 | cagaccccct gtagcgttta gcaggatggg ggccccaggt actgtggaga gcatagtcca |
| 2401 | gcctgggcat ttgtctccta gcagcctaca ctggctctgc tgagctgggc ctgggtgctg |
| 2461 | aaagccagga tttggggcta ggcgggaaga tgttcgccca attgcttggg gggttggggg |
| 2521 | gatggaaaag gggagcacct ctaggctgcc tggcagcagt gagccctggg cctgtggcta |
| 2581 | cagccaggga accccacctg gacacatggc cctgcttcta agccccccag ttaggcccaa |
| 2641 | aggaatggtc cactgagggc ctcctgctct gcctgggctg ggccaggggc tttgaggaga |
| 2701 | gggtaaacat aggcccggag atggggctga cacctcgagt ggccagaata tgcccaaacc |
| 2761 | ccggcttctc ccttgtccct aggcagaggg gggtcccttc ttttgttccc tctggtcacc |
| 2821 | acaatgcttg atgccagctg ccataggaag agggtgctgg ctggccatgg tggcacacac |
| 2881 | ctgtcctccc agcactttgc agggctgagg tggaaggacc gcttaagccc aggtgttcaa |
| 2941 | ggctgctgtg agctgtgttc gagccactac actccagcct ggggacggag caaaactttg |
| 3001 | cctcaaaaca aattttaaaa agaaagaaag aaggaaagag ggtatgtttt tcacaattca |
| 3061 | tgggggcctg catggcagga gtggggacag gacacctgct gttcctggag tcgaaggaca |
| 3121 | agcccacagc ccagattccg gttctcccaa ctcaggaaga gcatgccctg ccctctgggg |
| 3181 | aggctggcct ggccccagcc ctcagctgct gaccttgagg cagagacaac ttctaagaat |
| 3241 | ttggctgcca gaccccaggc ctggctgctg ctgtgtggag agggaggcgg cccgcagcag |
| 3301 | aacagccacc gcacttcctc ctcagcttcc tctggtgcgg ccctgccctc tcttctctgg |
| 3361 | acccttttac aactgaacgc atctgggctt cgtggtttcc tgttttcagc gaaatttact |
| 3421 | ctgagctccc agttccatct tcatccatgg ccacaggccc tgcctacaac gcactaggga |
| 3481 | cgtccctccc tgctgctgct ggggaggggc aggctgctgg agccgccctc tgagttgccc |
| 3541 | gggatggtag tgcctctgat gccagccctg gtggctgtgg gctggggtgc atgggagagc |
| 3601 | tgggtgcgag aacatggcgc ctccaggggg cgggaggagc actaggggct ggggcaggag |
| 3661 | gctcctggag cgctggattc gtggcacagt ctgaggccct gagagggaaa tccatgcttt |
| 3721 | taagaactaa ttcattgtta ggagatcaat caggaattag gggccatctt acctatctcc |
| 3781 | tgacattcac agtttaatag agacttcctg cctttattcc ctcccaggga gaggctgaag |
| 3841 | gaatggaatt gaaagcacca tttggagggt tttgctgaca cagcggggac tgctcagcac |
| 3901 | tccctaaaaa cacaccatgg aggccactgg tgactgctgg tgggcaggct ggccctgcct |
| 3961 | gggggagtcc gtggcgatgg gcgctggggt ggaggtgcag gagccccagg acctgctttt |
| 4021 | caaaagactt ctgcctgacc agagctccca ctacatgcag tggcccaggg cagaggggct |
| 4081 | gatacatggc ctttttcagg gggtgctcct cgcggggtgg acttgggagt gtgcagtggg |
| 4141 | acagggggct gcaggggtcc tgccaccacc gagcaccaac ttggcccctg gggtcctgcc |
| 4201 | tcatgaatga ggccttcccc agggctggcc tgactgtgct gggggctggg ttaacgtttt |
| 4261 | ctcagggaac cacaatgcac gaaagaggaa ctggggttgc taaccaggat gctgggaaca |
| 4321 | aaggcctctt gaagcccagc cacagcccag ctgagcatga ggcccagccc atagacggca |
| 4381 | caggccacct ggcccattcc ctgggcattc cctgctttgc attgctgctt ctcttcaccc |
| 4441 | catggaggct atgtcaccct aactatcctg gaatgtgttg agagggattc tgaatgatca |
| 4501 | atatagcttg gtgagacagt gccgagatag atagccatgt ctgccttggg cacgggagag |
| 4561 | ggaagtggca gcatgcatgc tgtttcttgg ccttttctgt tagaatactt ggtgctttcc |
| 4621 | aacacacttt cacatgtgtt gtaacttgtt tgatccaccc ccttccctga aaatcctggg |
| 4681 | aggttttatt gctgccattt aacacagagg gcaatagagg ttctgaaagg tctgtgtctt |
| 4741 | gtcaaaacaa gtaaacggtg gaactacgac taaa |

Bold = VISTA extracellular domain containing IgV domain (235-720)
VISTA cDNA nucleotide sequence (mouse) (SEQ ID NO: 20)
ACCESSION XR_380449

| | |
|---|---|
| 1 | catgaaggag ggcggagctg ggaggctgca aggctgctgt ggatctaggg aagaggaacc |
| 61 | acagaaaagc taaaggagtc acctctgtct ggtagcagcc ttcaaggtca ccactccata |
| 121 | ttctctctat gtgtgtcccg agggacagaa tgccaccctc acctgcagga ttctgggccc |
| 181 | cgtgtccaaa gggcacgatg tgaccatcta caagacgtgg tacctcagct cacgaggcga |
| 241 | ggtccagatg tgcaaagaac accggcccat acgcaacttc acattgcagc accttcagca |
| 301 | ccacggaagc cacctgaaag ccaacgccag ccatgaccag ccccagaagc atgggctaga |
| 361 | gctagcttct gaccaccacg gtaacttctc tatcaccctg cgcaatgtga ccccaaggga |
| 421 | cagcggcctc tactgctgtc tagtgataga attaaaaaac caccacccag aacaacggtt |
| 481 | ctacgggtcc atggagctac aggtacaggc aggcaaaggc tcggggtcca catgcatggc |
| 541 | gtctaatgag caggacagtg acagcatcac ggctgcggcc ctggccaccg gcgcctgcat |
| 601 | cgtgggaatc ctctgcctcc cccttatcct gctgctggtc tataagcaga gacaggtggc |
| 661 | ctctcaccgc cgtgcccagg agttggtgag gatggacagc aacacccaag gaatcgaaaa |
| 721 | cccaggcttc gagaccactc cacccttcca ggggatgcct gaggccaaga ccaggccgcc |
| 781 | actgtcctat gtggcccagc ggcaaccttc ggagtcagga cggtacctgc tctctgaccc |
| 841 | cagcacacct ctgtcgcctc caggccctgg ggacgtcttt ttcccatccc tagatccagt |
| 901 | ccctgactcc cctaactctg aagccatcta aaccagctgg ggaaccatga accatggtac |
| 961 | ctgggtcagg gatatgtgca cttgatctat ggctggccct tggacagtct tttaggcact |
| 1021 | gactccagct tccttgctcc tgctctgagc ctagactctg cttttacaag atgcacagac |
| 1081 | cctcccctat ctctttcaga cgctacttgg ggggcaggga gaagatgttg gattgctcat |
| 1141 | tgctgttctc aagatcttgg gatgctgagt tctccctaga gacttgactt cgacagccac |
| 1201 | agatgtcaga tgacctgcat cctatgaacg tccggcttgg caagagcctt tcttcatgga |
| 1261 | aaccagtagc ccggagggga tgagggaact tggagccaca acatggaagg gacctgggct |
| 1321 | gacaattgtc acaggaaagg ccacccactg gacacctgcc acactgctgg ctcagaagtc |
| 1381 | tttgccatat gcatatccca tgaggactgc acacttggag gtgaaggtga agctgccggt |
| 1441 | ctgcctcaag gacagggacc cacatgtgga acttgtggtg ctggctcaag ttgggccttc |
| 1501 | cattgccagg cttctactgg aatatacgtc aacgactacc cttcaaagcc accccactaa |
| 1561 | aagccatggc tcctgggatc tgactcatgg tagcaagtct caggacaccc cacactccag |
| 1621 | atttcttcat caggactgtg tgattaagca tcttctatta ttcttttttc cacgggactg |
| 1681 | gctgcagatg aacgtgaccc agtgtcaatg agggtcctgc acatggtagc tgacaataac |
| 1741 | cagatgcccc cagagacttc cctggtttgt tatggatgcc tgggaagctg acactggccc |
| 1801 | ctcccccaaa tgtatgtacc aaggctgtac ctgccatgat gttctccagc acagacacga |
| 1861 | agtaggccgg cttgctgaag gtgggagggt tgtcgttctc atcctggaag gaaagcacat |
| 1921 | ggatcagcat gtcgggaagg tactcggcca actttgggta aaagtaggaa ccgaggggca |
| 1981 | gtttgggaag ggtggccctg agggtctctg gcctctatgt atcaccagag gctagggtat |
| 2041 | tatgaaagcc aggagtccct ctgccaggtc agttgaagga taggttgggt ggtctttggg |
| 2101 | gaagcatgta agggttgggg cgggggatga aatgagattt agagacagac actgagtaag |
| 2161 | agagcagtca gctcgtctcc agctcccagt ctcccaccta acagctgcac tccaagctta |
| 2221 | ctccagaggc ccagtccctt agttctttcg ttcctatcca gcgtccacct gatctctccc |
| 2281 | agctgagtca agacacagtg aaaggttcta gaagctctgc tagaatgaag agagagggct |
| 2341 | ggagagatgg cttaatggtc aagagcactg gctgctggtc cagaggactt gggttcaatt |
| 2401 | cccagcatct acaactctct gtaacttcag ttcccaggga tctgatgcct ttttctggcc |
| 2461 | tctgccattt ggcactcagc tggtacagag acaatccatt catgtaagca aaacacccaa |
| 2521 | tcacatatat ttttttaatt aatttaaaaa aggtgaggaa gaggattcaa gagagggctc |
| 2581 | agcagtaaag aacactgtct gctcttccag aggttcccgg ttgaatccca gcacccacat |
| 2641 | ggcagatcac aactatctgt aatttcagtt ccaggggatc tggtaccctt gcacagacat |
| 2701 | acaatgcagg gaaaacatca atgtacattt aaaaaaatct ttttttaaag ggcgaaggag |
| 2761 | aaaggcacat ctagtagcac agatatgaaa gtgtacaggg gcaggtagtg gtcaggctgg |
| 2821 | tcccagagcc tggactggct gtagctgggg ttggacagcc agcttggtct accagcttcg |
| 2881 | catcctctgg atcaccctgg tctgcagcct ggtacctgcc agttccttcc tttggctcag |
| 2941 | cctgcagcag ctagggatgg gagtgggaag cctggctgag gggctagacc aggtagagat |
| 3001 | gagccctgtg ttggggagaa cccgtgccag gtgcagccca cagctcccta gactcttcaa |
| 3061 | tcagagaagc aagcctccca tctttgggac tctcaccaga gccaacttct gccatcgctc |
| 3121 | accgggaatt cctcccagtt gccttcttca ctgaagacgc tctggctccc ctgactccag |
| 3181 | ctcaactccg tgctcccctc ctcagctcaa cagagcattg gaggagctca ggagctggct |
| 3241 | gagtgttccg cctttctccc catgttgcaa tattcaatgg gccccactac taggtcctca |
| 3301 | gagctcaggg acctggagac tcctccaggg gacagagttt gcatttgctg aactcttttg |
| 3361 | ctacggtttc ttctgcatga gcaagcagga aaaggatgag accatgctgc ttggcccgcc |
| 3421 | tctccccaga ctcgggacat ccccagcctg ctgcatggtg tgttcccaga attgatttgc |
| 3481 | tccatttctt cccaaatctg tcccctcgct ctgtcctgcg tacaggctca ctcccttccc |
| 3541 | aaggagctgg gagctttggg agtctcctga cacccaaggt ccccacaggt cacccaggac |
| 3601 | ctcacccagg ctgctggcat gagctcaagt gctttgggta tacactggga ggcttctgct |
| 3661 | cagctgcacg aagcaacgca gagaatgtca gctacggagc acgaggcaga aacttattag |
| 3721 | cggtgatatt tccccggatt ctggcgctgt acttgctatt agttctaatt cagtggctcc |
| 3781 | ctctataaaa gcttattgtc ccctaatggg aaatct |

Bold = VISTA extracellular domain containing IgV domain (101-574)
Full length COMP cDNA nucleotide sequence (human) (SEQ ID NO: 21)
ACCESSION NM_000095

| | |
|---|---|
| 1 | agaaagcgag cagccaccca gctccccgcc accgccatgg tccccgacac cgcctgcgtt |
| 61 | cttctgctca ccctggctgc cctcggcgcg tccggacagg gccagagccc gttgggctca |
| 121 | gacctgggcc cgcagatgct tcgggaactg caggaaacca acgcggcgct gcaggacgtq |
| 181 | cgggagctgc tgcggcagca ggtcagggag atcacgttcc tgaaaaacac ggtgatggag |
| 241 | tgtgacgcgt gcgggatgca gcagtcagta cgcaccggcc tacccagcgt gcggcccctg |
| 301 | ctccactgcg cgcccggctt ctgcttcccc ggcgtggcct gcatccagac ggagagcggc |
| 361 | gcgcgctgcg gcccctgccc cgcgggcttc acgggcaacg gctcgcactg caccgacgtc |
| 421 | aacgagtgca acgcccaccc ctgcttcccc cgagtccgct gtatcaacac cagcccgggg |
| 481 | ttccgctgcg aggcttgccc gccggggtac agcggcccca cccaccaggg cgtggggctg |
| 541 | gctttcgcca aggccaacaa gcaggtttgc acggacatca acgagtgtga gaccgggcaa |
| 601 | cataactgcg tccccaactc cgtgtgcatc aacacccggg gctccttcca gtgcggcccg |
| 661 | tgccagcccg gcttcgtggg cgaccaggcg tccggctgcc agcggcgcgc acagcgcttc |
| 721 | tgccccgacg gctcgcccag cgagtgccac gagcatgcag actgcgtcct agagcgcgat |
| 781 | ggctcgcggt cgtgcgtgtg tgccgttggc tgggccggca acgggatcct ctgtggtcgc |
| 841 | gacactgacc tagacggctt cccggacgag aagctgcgct gcccggagcg ccagtgccgt |
| 901 | aaggacaact gcgtgactgt gcccaactca gggcaggagg atgtggaccg cgatggcatc |
| 961 | ggagacgcct gcgatccgga tgccgacggg gacggggtcc ccaatgaaaa ggacaactgc |
| 1021 | ccgctggtgc ggaacccaga ccagcgcaac acggacgagg acaagtgggg cgatgcgtgc |
| 1081 | gacaactgcc ggtcccagaa gaacgacgac caaaaggaca cagaccagga cggccggggc |
| 1141 | gatgcgtgcg acgacgacat cgacggcgac cggatccgca accaggccga caactgccct |
| 1201 | agggtaccca actcagacca gaaggacagt gatggcgatg gtatagggga tgcctgtgac |
| 1261 | aactgtcccc agaagagcaa cccggatcag gcggatgtgg accacgactt tgtgggagat |
| 1321 | gcttgtgaca gcgatcaaga ccaggatgga gacggacatc aggactctcg ggacaactgt |
| 1381 | cccacggtgc ctaacagtgc ccaggaggac tcagaccacg atggccaggg tgatgcctgc |
| 1441 | gacgacgacg acgacaatga cggagtccct gacagtcggg acaactgccg cctggtgcct |
| 1501 | aaccccggcc aggaggacgc ggacagggac ggcgtgggcg acgtgtgcca ggacgacttt |
| 1561 | gatgcagaca aggtggtaga caagatcgac gtgtgtccgg agaacgctga agtcacgctc |
| 1621 | accgacttca gggccttcca gacagtcgtg ctggacccgg agggtgacgc gcagattgac |
| 1681 | cccaactggg tggtgctcaa ccagggaagg gagatcgtgc agacaatgaa cagcgaccca |
| 1741 | ggcctggctg tgggttacac tgccttcaat ggcgtggact tcgagggcac gttccatgtg |
| 1801 | aacacggtca cggatgacga ctatgcgggc ttcatctttg gctaccagga cagctccagc |
| 1861 | ttctacgtgg tcatgtggaa gcagatggag caaacgtatt ggcaggcgaa ccccttccgt |
| 1921 | gctgtggccg agcctggcat ccaactcaag gctgtgaagt cttccacagg ccccggggaa |
| 1981 | cagctgcgga acgctctgtg gcatacagga gacacagagt cccaggtgcg gctgctgtgg |
| 2041 | aaggacccgc gaaacgtggg ttggaaggac aagaagtcct atcgttggtt cctgcagcac |
| 2101 | cggccccaag tgggctacat cagggtgcga ttctatgagg gccctgagct ggtggccgac |
| 2161 | agcaacgtgg tcttggacac aaccatgcgg ggtggccgcc tgggggtctt ctgcttctcc |
| 2221 | caggagaaca tcatctgggc caacctgcgt taccgctgca atgacaccat cccagaggac |
| 2281 | tatgagaccc atcagctgcg gcaagcctag ggaccagggt gaggacccgc cggatgacag |
| 2341 | ccaccctcac cgcggctgga tgggggctct gcacccagcc ccaaggggtg gccgtcctga |
| 2401 | gggggaagtg agaagggctc agagaggaca aaataaagtg tgtgtgcagg gaaaaaaaaa |
| 2461 | aaaaaaaaaa a |

Bold = Pentamerization domain (121-255)
COMP cDNA nucleotide sequence (mouse) (SEQ ID NO: 22)
ACCESSION NM_016685

| | |
|---|---|
| 1 | gacagcagct gcagctccgc cgccatgggc cccactgcct gcgttctagt gctcgccctg |
| 61 | gctatcctgc gggcgacagg ccagggccag atcccgctgg gtggagacct ggccccacag |
| 121 | atgctgcgag aacttcagga gactaatgcg gcgctgcaag acgtgagaga gctgttgcga |
| 181 | cagcaggtca aggagatcac cttcctgaag aatacggtga tggaatgtga tgcttgcgga |
| 241 | atgcagcccg cacgcactcc aggcctgagc gtgcggccag tgccgctctg cgcacccggc |
| 301 | tcctgcttcc ccggcgtagt ctgctccgag acagctacgg gcgcgcgctg cggcccctgc |
| 361 | cctcctggct acaccggcaa cggctcgcac tgcaccgacg ttaatgagtg caatgctcac |
| 421 | ccctgtttcc cgcgggtgcg gtgcatcaat accagccctg gctttcactg cgaagcctgt |
| 481 | ccccctgggt tcagcggacc cacccacgag ggcgtgggac tgaccttcgc taagtccaac |
| 541 | aaacaagttt gcacggatat taatgagtgt gagaccgggc agcacaattg cgttcccaac |
| 601 | tccgtgtgcg tcaacacccg gggctccttc cagtgcggcc cctgccagcc cggtttcgtg |
| 661 | ggcgaccaga cgtcaggctg ccagcggcgt gggcagcact tctgccccga tgggtcaccc |
| 721 | agcccgtgcc atgagaaagc aaactgcgtc ctggagcggg atggctcgag gtcttgcgtg |
| 781 | tgtgcagttg gctgggccgg caacgggctc ctgtgcggcc gcgacacgga cctggacggt |
| 841 | tttcctgacg agaagcttcg ctgctcagag cgccagtgtc gcaaggacaa ctgcgtgacg |
| 901 | gtgcccaatt cggggcagga ggatgtggac cgggacggca tcggagatgc ttgtgacccg |
| 961 | gatgcggacg gggatggagt ccctaacgag caagacaatt gcccgctggt tcgaaaccca |
| 1021 | gaccagcgta actcggacag tgataagtgg ggagatgcct gcgacaactg ccggtccaag |
| 1081 | aagaatgacg atcagaaaga tacagacctg gatggccggg gcgatgcctg cgacgacgac |
| 1141 | atagatggcg accgaatacg aaatgtagct gacaactgtc cccgggtgcc caactttgac |
| 1201 | cagagtgaca gtgatggtga tggtgttggg gatgcctgtg acaactgtcc ccagaaagat |
| 1261 | aacccagacc agagggatgt ggaccacgac tttgtgggtg atgcctgtga tagtgaccaa |
| 1321 | gaccaggatg gggatggtca ccaggactcc cgggacaact gccccacagt acccaacagt |
| 1381 | gcccagcagg actcagatca tgatggcaag ggcgatgcct gtgatgacga tgatgacaat |
| 1441 | gacggagttc ctgatagccg ggacaactgc cgcttggtgc ctaaccctgg ccaagaggac |
| 1501 | aatgaccggg atggcgtggg tgacgcgtgt cagggtgact tcgatgctga caaggttata |
| 1561 | gacaagatcg atgtgtgccc cgagaacgcc gaggtcaccc tcaccgactt cagggccttc |
| 1621 | cagacggttg tgttggaccc cgagggtgat gcgcagatcg atcccaactg ggtggtgctc |
| 1681 | aatcagggaa tggagatcgt tcagaccatg aacagtgacc ctggcctggc tgtgggttac |
| 1741 | acagccttca acggcgtgga cttcgagggc acattccatg taaacaccgc cactgatgat |
| 1801 | gactatgctg gtttcatctt cggctaccaa gacagctcca gtttctacgt agtcatgtgg |
| 1861 | aaacagatgg agcagacgta ctggcaggcc aatcccttcc gggctgtggc tgagccaggg |
| 1921 | attcagctca aggctgtcaa gtcctctaca ggtcccgggg aacagctccg aaacgcactg |
| 1981 | tggcacacgg gggacacagc atcccaggtg cggctgctgt ggaaggatcc tcgaaacgtg |
| 2041 | ggctggaagg ataaaacatc ctaccgctgg ttcctgcagc accggcctca agttggctac |
| 2101 | atcagggtgc ggttctatga gggtcctgag ctagtagctg acagcaatgt ggtgttggac |
| 2161 | acggccatgc gtggtggccg cctgggtgtc ttctgcttct cccaagagaa catcatctgg |
| 2221 | gctaacctgc gctaccgttg caatgataca atccctgagg actacgagag tcaccggctg |
| 2281 | cagagagtct agggaccagt ggggtcccgc tgcctgatgg actgtggtgg cacaagctac |
| 2341 | gggtgtgtgt gtgggggggt ctggcatccc tctgaagggg tgtctggcct ggggaggaga |
| 2401 | ggcaaataaa gtacgtatgt gggggaaaaa aaaaaaaaaa aaaaaaa |

Bold = Pentamerization domain (105-240)
Codon Optimized VISTA.COMP DNA sequence (human) (SEQ ID NO: 23)

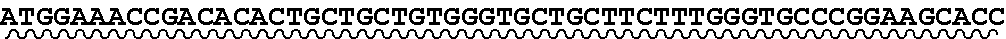

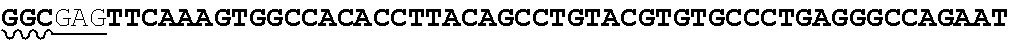

GTGACCCTGACCTGTAGACTGCTGGGCCCCGTGGATAAGGGCCACGACGTGACCTTT
TACAAGACCTGGTACAGATCCAGCAGAGGCGAGGTGCAGACCTGCTCTGAGAGAAG
GCCCATCCGGAACCTGACCTTCCAGGACCTGCATCTGCACCACGGTGGACATCAGGC
CGCCAATACCTCTCATGATCTGGCCCAGAGACACGGCCTGGAAAGCGCCTCTGATCA
CCACGGCAACTTCAGCATCACCATGCGGAATCTGACCCTGCTGGACAGCGGCCTGTA
CTGCTGTCTGGTGGTGGAAATCAGACACCACCACAGCGAGCACAGAGTGCACGGCG
CTATGGAACTGCAGGTCCAGACAGGCAAGGACGCCCCTAGCAATTGCGTGGTGTACC
CTAGCAGCAGCCAGGACAGCGAGAATATCACCGCCGAGTTTGGCTCTGGCCCTGGA

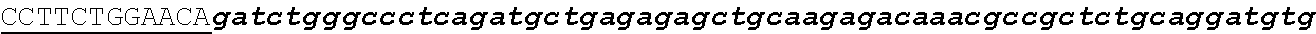

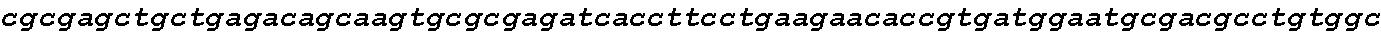

GGATCTCCTCAGCCTCAGAGCGAGAACCTGTACTTCCAAGGCGGCCCTCAACCTCAA

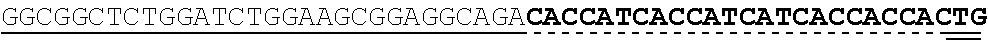

A
wavy underline = DNA sequencing encoding IgKappa secretion signal
underlined (gray) = Spacer sequences
uppercase, no underline (black) = VISTA extracellular domain
lower case italicized = COMP pentamerization domain
double underlined = stop codon
stippled underline = DNA sequence encoding a his-tag
VISTA.COMP amino acid sequence (human) (SEQ ID NO: 24)

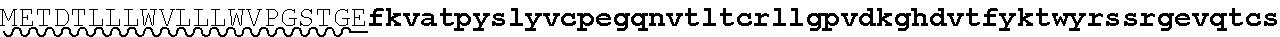

errpirnltfqdlhlhhgghqaantshdlaqrhglesasdhhgnfsitmrnltlldsglycclvveirhhhsehrvhgam
elqvqtgkdapsncvvypsssqdsenitaEFGSGPGPSGTdlgpqmlrelqetnaalqdvrellrqqvreitflkntvm

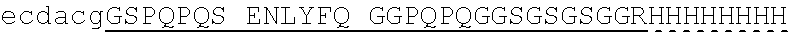

wavy underline = IgKappa secretion signal
lower case bold (black) = VISTA extracellular domain containing IgV domain
lower case italicized = COMP pentamerization domain
stippled underline = Histidine tag
underlined (gray) = Spacer sequences
B7-H4 extracellular domain amino acid sequence (human) (SEQ ID NO: 25)
ISGRHSITVTTVASAGNIGEDGILSCTFEPDIKLSDIVIQWLKEGVLGLVHEFK
EGKDELSEQDEMFRGRTAVFADQVIVGNASLRLKNVQLTDAGTYKCYIITSKGKGNANLE
YKTGAFSMPEVNVDYNASSETLRCEAPRWFPQPTVVWASQVDQGANFSEVSNTSFELNS
E
NVTMKVVSVLYNVTINNTYSCMIENDIAKATGDIKVTESEIKRRSHLQLLNSKAS
B7-H4 extracellular domain cDNA sequence (human) (SEQ ID NO: 26)
atttcagggagacactccatcacagtcactactgtcgcctcagctgggaacattggggaggatggaatcctgagctgcacttttga
acctgacatcaaactttctgatatcgtgatacaatggctgaaggaaggtgttttaggcttggtccatgagttcaaagaaggcaaag
atgagctgtcggagcaggatgaaatgttcagaggccggacagcagtgtttgctgatcaagtgatagttggcaatgcctctttgcgg
ctgaaaaacgtgcaactcacagatgctggcacctacaaatgttatatcatcacttctaaaggcaaggggaatgctaaccttgagt
ataaaactggagccttcagcatgccggaagtgaatgtggactataatgccagctcagagaccttgcggtgtgaggctccccgat
ggttcccccagcccacagtggtctgggcatcccaagttgaccagggagccaacttctcggaagtctccaataccagctttgagct
gaactctgagaatgtgaccatgaaggttgtgtctgtgctctacaatgttacgatcaacaacacatactcctgtatgattgaaaatgac
attgccaaagcaacaggggatatcaaagtgacagaatcggagatcaaaaggcggagtcacctacagctgctaaactcaaag
gcttct
B7-H4 extracellular domain mRNA sequence (human) (SEQ ID NO: 27)
auuucagggagacacuccaucacagucacuacugucgccucagcugggaacauuggggaggauggaauccugagc
ugcacuuuugaaccugacaucaaacuuucugauaucgugauacaauggcugaaggaagguguuuuaggcuugguc
caugaguucaaagaaggcaaagaugagcugucggagcaggaugaaauguucagaggccggacagcaguguuugc
ugaucaagugauaguuggcaaugccucuuugcggcugaaaaacgugcaacucacagaugcuggcaccuacaaaug
uuauaucaucacuucuaaaggcaaggggaaugcuaaccuugaguauaaaacuggagccuucagcaugccggaagu
gaauguggacuauaaugccagcucagagaccuugcggugugaggcuccccgaugguucccccagcccacagugguc
ugggcaucccaaguugaccagggagccaacuucucggaagucuccaauaccagcuuugagcugaacucugagaau
gugaccaugaagguugugucugugcucuacaauguuacgaucaacaacacauacuccuguaugauugaaaaugac
auugccaaagcaacaggggauaucaaagugacagaaucggagaucaaaaggcggagucaccuacagcugcuaaac
ucaaaggcuucu
B7-H4 full length amino acid sequence (human) (SEQ ID NO: 28)
ACCESSION Q7Z7D3
MASLGQILFWSIISIIIILAGAIALIIGFGISGRHSITVTTVASAGNIGEDGILSCTFEP
DIKLSDIVIQWLKEGVLGLVHEFKEGKDELSEQDEMFRGRTAVFADQVIVGNASLRLKNV
QLTDAGTYKCYIITSKGKGNANLEYKTGAFSMPEVNVDYNASSETLRCEAPRWFPQPTVV
WASQVDQGANFSEVSNTSFELNSENVTMKVVSVLYNVTINNTYSCMIENDIAKATGDIKV
TESEIKRRSHLQLLNSKASLCVSSFFAISWALLPLSPYLMLK
B7-H4 full length cDNA nucleotide sequence (human) (SEQ ID NO: 29)
ACCESSION AY280972

| | |
|---|---|
| 1 | atggcttccc tggggcagat cctcttctgg agcataatta gcatcatcat tattctggct |
| 61 | ggagcaattg cactcatcat tggctttggt atttcaggga gacactccat cacagtcact |
| 121 | actgtcgcct cagctgggaa cattggggag gatggaatcc tgagctgcac ttttgaacct |
| 181 | gacatcaaac tttctgatat cgtgatacaa tggctgaagg aaggtgtttt aggcttggtc |
| 241 | catgagttca aagaaggcaa agatgagctg tcggagcagg atgaaatgtt cagaggccgg |
| 301 | acagcagtgt ttgctgatca agtgatagtt ggcaatgcct ctttgcggct gaaaaacgtg |
| 361 | caactcacag atgctggcac ctacaaatgt tatatcatca cttctaaagg caaggggaat |
| 421 | gctaaccttg agtataaaac tggagccttc agcatgccgg aagtgaatgt ggactataat |
| 481 | gccagctcag agaccttgcg gtgtgaggct ccccgatggt tcccccagcc cacagtggtc |
| 541 | tgggcatccc aagttgacca gggagccaac ttctcggaag tctccaatac cagctttgag |
| 601 | ctgaactctg agaatgtgac catgaaggtt gtgtctgtgc tctacaatgt tacgatcaac |
| 661 | aacacatact cctgtatgat tgaaaatgac attgccaaag caacagggga tatcaaagtg |
| 721 | acagaatcgg agatcaaaag gcggagtcac ctacagctgc taaactcaaa ggcttctctg |
| 781 | tgtgtctctt ctttctttgc catcagctgg gcacttctgc ctctcagccc ttacctgatg |
| 841 | ctaaaataa |

B7-H4.COMP amino acid sequence (human B7-H4.COMP) (SEQ ID NO: 30)

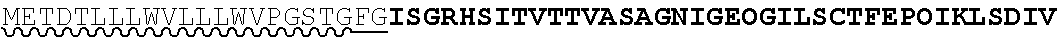

IQWLKEGVLGLVHEFKEGKDELSEQDEMFRGRTAVFADQVIVGNASLRLKNVQLTDAGT
Y
KCYIITSKGKGNANLEYKTGAFSMPEVNVDYNASSETLRCEAPRWFPQPTVVVVASQVDQ
G
ANFSEVSNTSFELNSENVTMKVVSVLYNVTINNTYSCMIENDIAKATGDIKVTESEIKRR
SHLQLLNSKAEFGSGPGPSGTdlapqmlrelqetnaalqdvrellrqqvkeitflkntvm

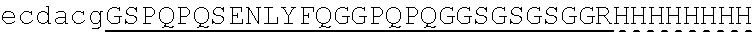

wavy underline = IgKappa secretion signal
Bold (black) = B7-H4 extracellular domain containing
lower case italicized = COMP pentamerization domain
stippled underline = Histidine tag
underlined (gray) = Spacer sequences
B7-H4.COMP codon optimized DNA sequence (human B7-H4) (SEQ ID NO: 31)
Atggagacagatacactcctgctctgggtcctcctgctgtgggtgcctggaagcacgggattcggcatttcagggcggcacagc
atcacagtcactacggtagcgagtgccgggaatattggggaagacggaatcctttcatgtaccttcgaacccgacataaagttgt
cagatatcgtgatccaatggcttaaagaaggagtcttgggactcgttcacgagtttaaggaaggcaaagatgaactcagtgaac
aagacgagatgtttaggggacggacagcggtgtttgctgatcaggtcattgtcggcaatgccagcctgcgccttaaaaatgttcag
ctcaccgatgcggggacctataagtgttatattatcacgtccaagggcaagggtaacgcgaacctcgagtacaaaacaggagc
tttttctatgcctgaagtgaatgtagactataatgcttcaagcgaaactcttcgatgcgaagcgccacgctggtttccgcagcccact
gtagtgtgggcgtcccaagtcgatcagggagcaaacttcagcgaggtgtcaaacacgtcatttgagcttaactctgagaacgtaa
ccatgaaagtggtaagcgtgttgtataatgtcacaatcaataatacttattcctgtatgatcgaaaacgatatcgctaaagcaaccg
gcgacatcaaagttacggaatccgagattaagcgccgctcacacttgcaactccttaattccaaggccgaattcgggtccggccc
cggtcctagcgggactgacctggctccgcaaatgcttagggagcttcaggaaacaaatgccgccttgcaagacgtgcgagaac
tgctgagacaacaggttaaggagatcacattcctgaagaatacagttatggaatgtgatgcctgcggggggtcccctcagcccc
agtctgaaaatttgtatttccaggggggtccacagccccaaggtggttcaggctcaggctcaggtggccggcaccatcatcatcat
caccaccactaatga
B7-H4.COMP amino acid sequence (mouse) (SEQ ID NO: 32)

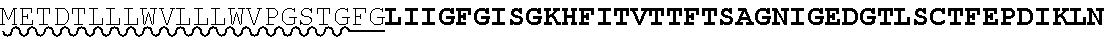

GIVIQWLKEGIKGLVHEFKEGKDDLSQQHEMFRGRTAVFADQVVVGNASLRLKNVQLTDA
GTYTCYIRTSKGKGNANLEYKTGAFSMPEINVDYNASSESLRCEAPRWFPQPTVAWASQ
VDQGANFSEVSNTSFELNSENVTMKVVSVLYNVTINNTYSCMIENDIAKATGDIKVTDSEVK
RRSQLQLLNSGEFGSGPGPSGTdlapqmlrelqetnaalqdvrellrqqvkeitflkntvm

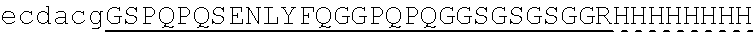

wavy underline = IgKappa secretion signal
Bold (black) = B7-H4 extracellular domain containing
lower case italicized = COMP pentamerization domain
stippled underline = Histidine tag
underlined (gray) = Spacer sequences
Codon optimized B7-H4.COMP DNA sequence (mouse) (SEQ ID NO: 33)

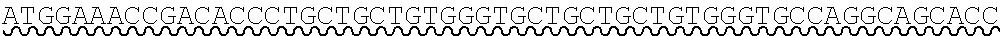

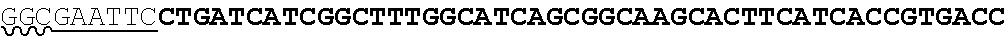

ACCTTTACCAGCGCCGGCAACATCGGCGAGGATGGCACACTGAGCTGCACCTTCGA
GCCCGACATCAAGCTGAACGGCATCGTGATCCAGTGGCTGAAAGAGGGCATCAAAG
GCCTGGTGCACGAGTTCAAAGAAGGCAAGGACGACCTGAGCCAGCAGCACGAGATG
TTCAGAGGCAGAACCGCCGTGTTCGCCGATCAGGTGGTCGTGGGAAATGCCAGCCT
GCGGCTGAAGAATGTGCAGCTGACAGACGCCGGCACCTACACCTGTTACATCCGGA
CCTCTAAAGGCAAGGGCAACGCCAACCTCGAGTACAAGACAGGCGCCTTCAGCATG
CCCGAGATCAACGTGGACTACAACGCCAGCAGCGAGAGCCTGAGATGCGAAGCCCC
TAGATGGTTCCCTCAGCCTACAGTGGCTTGGGCTAGTCAGGTTGACCAGGGCGCCAA
CTTTAGCGAGGTGTCCAACACCAGCTTCGAGCTGAACAGCGAGAACGTGACCATGAA
GGTGGTGTCCGTGCTGTACAATGTGACCATCAACAACACCTACAGCTGCATGATCGA
GAACGATATCGCCAAGGCCACCGGCGACATCAAAGTGACCGACAGCGAAGTGAAGC

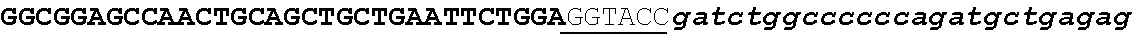

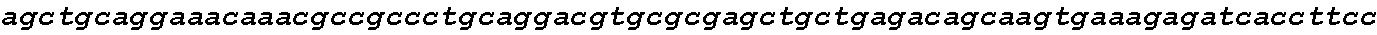

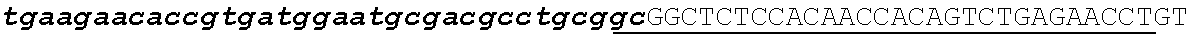

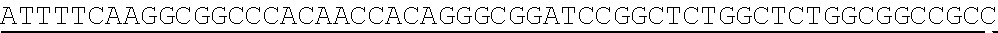

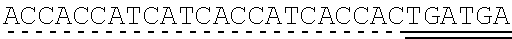

wavy underline = DNA sequencing encoding IgKappa secretion signal
uppercase, no underline (black) = PD-L1 extracellular domain
underlined (gray) = Spacer sequences
lower case italicized = COMP pentamerization domain
stippled underline = DNA sequence encoding a his-tag
double underlined = stop codon
B7-H4 full length amino acid sequence (mouse) (SEQ ID NO: 34)
ACCESSION Q7TSP5
MASLGQIIFWSIINIIIILAGAIALIIGFGISGKHFITVTTFTSAGNIGEDGTLSCTFEP
DIKLNGIVIQWLKEGIKGLVHEFKEGKDDLSQQHEMFRGRTAVFADQVVVGNASLRLKNV
QLTDAGTYTCYIRTSKGKGNANLEYKTGAFSMPEINVDYNASSESLRCEAPRWFPQPTVA
WASQVDQGANFSEVSNTSFELNSENVTMKVVSVLYNVTINNTYSCMIENDIAKATGDIKV
TDSEVKRRSQLQLLNSGPSPCVFSSAFVAGWALLSLSCCLMLR
B7-H4 full length cDNA nucleotide sequence (mouse) (SEQ ID NO: 35)
ACCESSION AY280973

| | |
|---|---|
| 1 | atggcttcct tggggcagat catcttttgg agtattatta acatcatcat catcctggct |
| 61 | ggggccatcg cactcatcat tggctttggc atttcaggca agcacttcat cacggtcacg |
| 121 | accttcacct cagctggaaa cattggagag gacgggaccc tgagctgcac ttttgaacct |
| 181 | gacatcaaac tcaacggcat cgtcatccag tggctgaaag aaggcatcaa aggtttggtc |
| 241 | cacgagttca aagaaggcaa agacgacctc tcacagcagc atgagatgtt cagaggccgc |
| 301 | acagcagtgt ttgctgatca ggtggtagtt ggcaatgctt ccctgagact gaaaaacgtg |
| 361 | cagctcacgg atgctggcac ctacacatgt tacatccgca cctcaaaagg caaagggaat |
| 421 | gcaaaccttg agtataagac cggagccttc agtatgccag agataaatgt ggactataat |
| 481 | gccagttcag agagtttacg ctgcgaggct cctcggtggt tcccccagcc cacagtggcc |
| 541 | tgggcatctc aagtcgacca aggagccaat ttctcagaag tctccaacac cagctttgag |
| 601 | ttgaactctg agaatgtgac catgaaggtc gtatctgtgc tctacaatgt cacaatcaac |
| 661 | aacacatact cctgtatgat tgaaaacgac attgccaaag ccaccgggga catcaaagtg |
| 721 | acagattcag aggtcaaaag gcgaagtcag ctgcagttgc tgaactctgg gccttccccg |
| 781 | tgtgtttttt cttctgcctt tgtggctggc tgggcactcc tatctctctc ctgttgcctg |
| 841 | atgctaagat ga |

PD-L1 extracellular domain amino acid sequence (human) (SEQ ID NO: 36)
FTVTVPKDLYVVEYGSNMTIECKFPVEKQLDLAALIVYWEMEDKNIIQFVHGEEDLKVQH
SSYRQRARLLKDQLSLGNAALQITDVKLQDAGVYRCMISYGGADYKRITVKVNAPYNKIN
QRILVVDPVTSEHELTCQAEGYPKAEVIVVTSSDHQVLSGKTTTTNSKREEKLFNVTSTLR
INTTTNEIFYCTFRRLDPEENHTAELVIPELPLAHPPNER
PD-L1 extracellular domain cDNA nucleotide sequence (human) (SEQ ID NO: 37)
catttactgtcacggttcccaaggacctatatgtggtagagtatggtagcaatatgacaattgaatgcaaattcccagtagaaaaa
caattagacctggctgcactaattgtctattgggaaatggaggataagaacattattcaatttgtgcatggagaggaagacctgaa
ggttcagcatagtagctacagacagagggcccggctgttgaaggaccagctctccctgggaaatgctgcacttcagatcacaga
tgtgaaattgcaggatgcaggggtgtaccgctgcatgatcagctatggtggtgccgactacaagcgaattactgtgaaagtcaat
gccccatacaacaaaatcaaccaaagaattttggttgtggatccagtcacctctgaacatgaactgacatgtcaggctgagggct
accccaaggccgaagtcatctggacaagcagtgaccatcaagtcctgagtggtaagaccaccaccaccaattccaagagag
aggagaagcttttcaatgtgaccagcacactgagaatcaacacaacaactaatgagattttctactgcacttttaggagattagatc
ctgaggaaaaccatacagctgaattggtcatcccagaactacctctggcacatcctccaaatgaaagg
PD-L1 full length amino acid sequence (human) (SEQ ID NO: 38)
ACCESSION Q9NZQ7
MRIFAVFIFMTYVVHLLNAFTVTVPKDLYVVEYGSNMTIECKFPVEKQLDLAALIVYVVEME
DKNIIQFVHGEEDLKVQHSSYRQRARLLKDQLSLGNAALQITDVKLQDAGVYRCMISYGG
ADYKRITVKVNAPYNKINQRILVVDPVTSEHELTCQAEGYPKAEVIVVTSSDHQVLSGKTT
TTNSKREEKLFNVTSTLRINTTTNEIFYCTFRRLDPEENHTAELVIPELPLAHPPNERTH
LVILGAILLCLGVALTFIFRLRKGRMMDVKKCGIQDTNSKKQSDTHLEET
PD-L1 full length nucleotide sequence (human) (SEQ ID NO: 39)
ACCESSION AF177937

| | |
|---|---|
| 1 | atgaggatat ttgctgtctt tatattcatg acctactggc atttgctgaa cgcatttact |
| 61 | gtcacggttc ccaaggacct atatgtggta gagtatggta gcaatatgac aattgaatgc |
| 121 | aaattcccag tagaaaaaca attagacctg gctgcactaa ttgtctattg ggaaatggag |
| 181 | gataagaaca ttattcaatt tgtgcatgga gaggaagacc tgaaggttca gcatagtagc |
| 241 | tacagacaga gggcccggct gttgaaggac cagctctccc tgggaaatgc tgcacttcag |
| 301 | atcacagatg tgaaattgca ggatgcaggg gtgtaccgct gcatgatcag ctatggtggt |
| 361 | gccgactaca agcgaattac tgtgaaagtc aatgccccat acaacaaaat caaccaaaga |
| 421 | attttggttg tggatccagt cacctctgaa catgaactga catgtcaggc tgagggctac |
| 481 | cccaaggccg aagtcatctg gacaagcagt gaccatcaag tcctgagtgg taagaccacc |
| 541 | accaccaatt ccaagagaga ggagaagctt ttcaatgtga ccagcacact gagaatcaac |
| 601 | acaacaacta atgagatttt ctactgcact tttaggagat tagatcctga ggaaaaccat |
| 661 | acagctgaat tggtcatccc agaactacct ctggcacatc ctccaaatga aaggactcac |
| 721 | ttggtaattc tgggagccat cttattatgc cttggtgtag cactgacatt catcttccgt |
| 781 | ttaagaaaag ggagaatgat ggatgtgaaa aaatgtggca tccaagatac aaactcaaag |
| 841 | aagcaaagtg atacacattt ggaggagacg taa |

PD-LI.COMP amino acid sequence (human) (SEQ ID NO: 40)

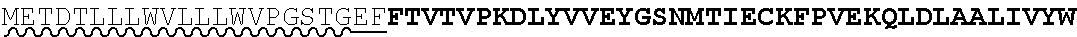

EMEDKNIIQFVHGEEDLKVQHSSYRQRARLLKDQLSLGNAALQITDVKLQDAGVYRCMIS
YGGADYKRITVKVNAPYNKINQRILVVDPVTSEHELTCQAEGYPKAEVIWTSSDHQVLSGK
TTTTNSKREEKLFNVTSTLRINTTTNEIFYCTFRRLDPEENHTAELVIPELPLAHPPNERGTdl
apqmlrelqetnaalqdvrellrqqvkeitflkntymecdacgGSPQPQSENLYFQGGPQPQGGSGSGSGGR

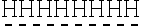

wavy underline = IgKappa secretion signal
Bold (black) = PD-L1 extracellular domain containing IgV domain
lower case italicized = COMP pentamerization domain
stippled underline = Histidine tag
underlined (gray) = Spacer sequences
Codon optimized PD-LI.COMP encoding nucleotide sequence (human) (SEQ ID NO: 41)

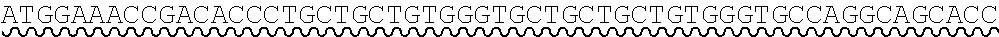

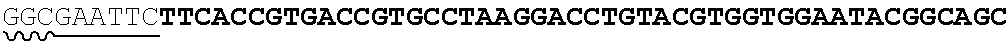

AACATGACCATCGAGTGCAAGTTCCCCGTGGAAAAGCAGCTGGATCTGGCCGCTCTG
ATCGTGTACTGGGAGATGGAAGATAAGAACATCATCCAGTTCGTGCACGGCGAAGAG
GACCTGAAGGTGCAGCACAGCAGCTACAGACAGAGAGCCAGACTGCTGAAGGACCA
GCTGAGCCTGGGAAATGCCGCTCTGCAGATCACCGACGTGAAGCTGCAAGATGCCG
GCGTGTACCGGTGCATGATCTCTTATGGCGGAGCCGACTACAAGCGGATCACCGTGA
AAGTGAACGCCCCTTACAACAAGATCAACCAGCGGATCCTGGTGGTGGACCCTGTGA
CATCTGAGCACGAGCTGACCTGTCAGGCCGAGGGATATCCTAAGGCCGAAGTGATCT
GGACCAGCAGCGATCACCAGGTGCTGAGCGGCAAGACCACCACCACAAACAGCAAG
CGGGAAGAGAAGCTGTTCAACGTGACCAGCACACTGCGGATCAACACAACCACCAA
CGAGATCTTCTACTGCACCTTTCGGCGGCTGGACCCCGAGGAAAATCACACAGCCGA

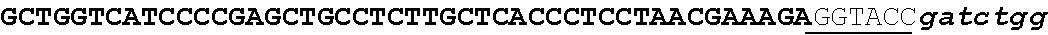

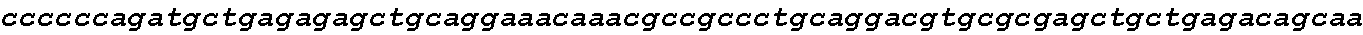

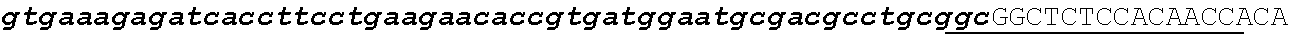

GTCTGAGAACCTGTATTTTCAAGGCGGCCCACAACCACAGGGCGGATCCGGCTCTGGC

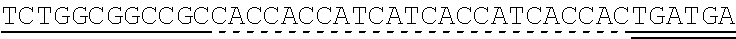

wavy underline = DNA sequencing encoding IgKappa secretion signal
uppercase, no underline (black) = PD-L1 extracellular domain
underlined (gray) = Spacer sequences
lower case italicized = COMP pentamerization domain
stippled underline = DNA sequence encoding a his-tag
double underlined = stop codon
PD-L1 extracellular domain amino acid sequence (mouse) (SEQ ID NO: 42)
FTITAPKDLYVVEYGSNVTMECRFPVERELDLLALVVYWEKEDEQVIQFVAGEEDLKPQH
SNFRGRASLPKDQLLKGNAALQITDVKLQDAGVYCCIISYGGADYKRITLKVNAPYRKIN
QRISVDPATSEHELICQAEGYPEAEVIVVTNSDHQPVSGKRSVTTSRTEGMLLNVTSSLRV
NATANDVFYCTFWRSQPGQNHTAELIIPELPATHPPQNR
PD-L1 extracellular domain cDNA nucleotide sequence (mouse) (SEQ ID NO: 43)
Tttactatcacggctccaaaggacttgtacgtggtggagtatggcagcaacgtcacgatggagtgcagattccctgtagaacggg
agctggacctgcttgcgttagtggtgtactgggaaaaggaagatgagcaagtgattcagtttgtggcaggagaggaggacctta
agcctcagcacagcaacttcagggggagagcctcgctgccaaaggaccagcttttgaagggaaatgctgcccttcagatcaca
gacgtcaagctgcaggacgcaggcgtttactgctgcataatcagctacggtggtgcggactacaagcgaatcacgctgaaagt
caatgccccataccgcaaaatcaaccagagaatttccgtggatccagccacttctgagcatgaactaatatgtcaggccgaggg
ttatccagaagctgaggtaatctggacaaacagtgaccaccaacccgtgagtgggaagagaagtgtcaccacttcccggaca
gaggggatgcttctcaatgtgaccagcagtctgagggtcaacgccacagcgaatgatgttttctactgtacgttttggagatcacag
ccagggcaaaaccacacagcggagctgatcatcccagaactgcctgcaacacatcctccacagaacagg
PD-L1 full length amino acid sequence (mouse) (SEQ ID NO: 44)
ACCESSION Q9EP73
MRIFAGIIFTACCHLLRAFTITAPKDLYVVEYGSNVTMECRFPVERELDLLALVVYVVEKE
DEQVIQFVAGEEDLKPQHSNFRGRASLPKDQLLKGNAALQITDVKLQDAGVYCCIISYGG
ADYKRITLKVNAPYRKINQRISVDPATSEHELICQAEGYPEAEVIVVTNSDHQPVSGKRSV
TTSRTEGMLLNVTSSLRVNATANDVFYCTFWRSQPGQNHTAELIIPELPATHPPQNRTHW
VLLGSILLFLIVVSTVLLFLRKQVRMLDVEKCGVEDTSSKNRNDTQFEET
PD-L1 full length cDNA nucleotide sequence (mouse) (SEQ ID NO: 45)
ACCESSION AF317088

| | |
|---|---|
| 1 | atgaggatat ttgctggcat tatattcaca gcctgctgtc acttgctacg ggcgtttact |
| 61 | atcacggctc caaaggactt gtacgtggtg gagtatggca gcaacgtcac gatggagtgc |
| 121 | agattccctg tagaacggga gctggacctg cttgcgttag tggtgtactg ggaaaaggaa |
| 181 | gatgagcaag tgattcagtt tgtggcagga gaggaggacc ttaagcctca gcacagcaac |
| 241 | ttcaggggga gagcctcgct gccaaaggac cagcttttga agggaaatgc tgcccttcag |
| 301 | atcacagacg tcaagctgca ggacgcaggc gtttactgct gcataatcag ctacggtggt |
| 361 | gcggactaca agcgaatcac gctgaaagtc aatgccccat accgcaaaat caaccagaga |
| 421 | atttccgtgg atccagccac ttctgagcat gaactaatat gtcaggccga gggttatcca |
| 481 | gaagctgagg taatctggac aaacagtgac caccaacccg tgagtgggaa gagaagtgtc |
| 541 | accacttccc ggacagaggg gatgcttctc aatgtgacca gcagtctgag ggtcaacgcc |
| 601 | acagcgaatg atgttttcta ctgtacgttt tggagatcac agccagggca aaaccacaca |
| 661 | gcggagctga tcatcccaga actgcctgca acacatcctc cacagaacag gactcactgg |
| 721 | gtgcttctgg gatccatcct gttgttcctc attgtagtgt ccacggtcct cctcttcttg |
| 781 | agaaaacaag tgagaatgct agatgtggag aaatgtggcg ttgaagatac aagctcaaaa |
| 841 | aaccgaaatg atacacaatt cgaggagacg taa |

PD-LI.COMP amino acid sequence (mouse) (SEQ ID NO: 46)

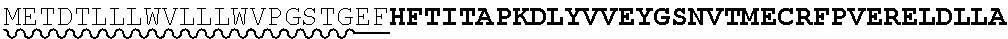

LVVYWEKEDEQVIQFVAGEEDLKPQHSNFRGRASLPKDQLLKGNAALQITDVKLQDAGV
Y
CCIISYGGADYKRITLKVNAPYRKINQRISVDPATSEHELICQAEGYPEAEVIWTNSDHQ
PVSGKRSVTTSRTEGMLLNVTSSLRVNATANDVFYCTFWRSQPGQNHTAELIIPELPATH
PPQNRGTdlapqmlrelqetnaalqdvrellrqqvkeitflkntymecdacgGSPQPQSENLYFQGGPQPQGG

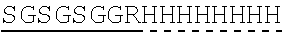

wavy underline = IgKappa secretion signal
Bold (black) = PD-L1 extracellular domain containing IgV domain
lower case italicized = COMP pentamerization domain
stippled underline = Histidine tag
underlined (gray) = Spacer sequences
PD-L1.COMP encodinq nucleotide sequence (mouse) (SEQ ID NO: 47)

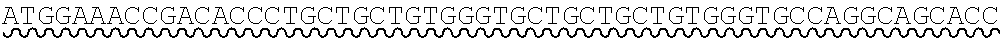

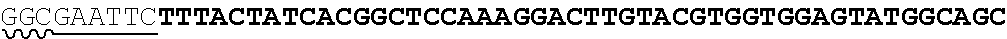

AACGTCACGATGGAGTGCAGATTCCCTGTAGAACGGGAGCTGGACCTGCTTGCGTTA
GTGGTGTACTGGGAAAAGGAAGATGAGCAAGTGATTCAGTTTGTGGCAGGAGAGGA
GGACCTTAAGCCTCAGCACAGCAACTTCAGGGGGAGAGCCTCGCTGCCAAAGGACC
AGCTTTTGAAGGGAAATGCTGCCCTTCAGATCACAGACGTCAAGCTGCAGGACGCAG
GCGTTTACTGCTGCATAATCAGCTACGGTGGTGCGGACTACAAGCGAATCACGCTGA
AAGTCAATGCCCCATACCGCAAAATCAACCAGAGAATTTCCGTGGATCCAGCCACTT
CTGAGCATGAACTAATATGTCAGGCCGAGGGTTATCCAGAAGCTGAGGTAATCTGGA
CAAACAGTGACCACCAACCCGTGAGTGGGAAGAGAAGTGTCACCACTTCCCGGACA
GAGGGGATGCTTCTCAATGTGACCAGCAGTCTGAGGGTCAACGCCACAGCGAATGAT
GTTTTCTACTGTACGTTTTGGAGATCACAGCCAGGGCAAAACCACACAGCGGAGCTG

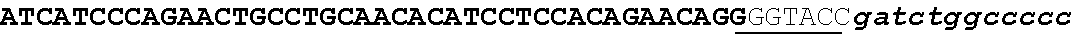

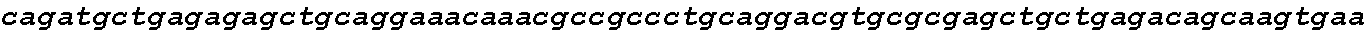

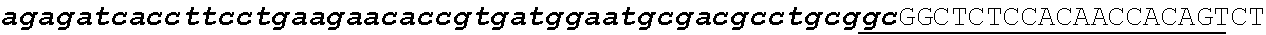

GAGAACCTGTATTTTCAAGGCGGCCCACAACCACAGGGCGGATCCGGCTCTGGCTCTG

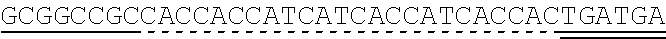

wavy underline = DNA sequencing encoding IgKappa secretion signal
uppercase, no underline (black) = PD-L1 extracellular domain
underlined (gray) = Spacer sequences
lower case italicized = COMP pentamerization domain
stippled underline = DNA sequence encoding a his-tag
double underlined = stop codon
ICOS-L extracellular domain cDNA nucleotide sequence (human) (SEQ ID NO: 48)
gatactcaggagaaggaagtcagagcgatggtaggcagcgacgtggagctcagctgcgcttgccctgaaggaagccgttttga
tttaaatgatgtttacgtatattggcaaaccagtgagtcgaaaaccgtggtgacctaccacatcccacagaacagctccttggaaa
acgtggacagccgctaccggaaccgagccctgatgtcaccggccggcatgctgcggggcgacttctccctgcgcttgttcaacg
tcaccccccaggacgagcagaagtttcactgcctggtgttgagccaatccctgggattccaggaggttttgagcgttgaggttaca
ctgcatgtggcagcaaacttcagcgtgcccgtcgtcagcgccccccacagcccctcccaggatgagctcaccttcacgtgtacat
ccataaacggctaccccaggcccaacgtgtactggatcaataagacggacaacagcctgctggaccaggctctgcagaatga
caccgtcttcttgaacatgcggggcttgtatgacgtggtcagcgtgctgaggatcgcacggacccccagcgtgaacattggctgct
gcatagagaacgtgcttctgcagcagaacctgactgtcggcagccagacaggaaatgacatcggagagagagacaagatca
cagagaatccagtcagtaccggcgagaaaaacgcggccacgtggtcc
ICOS-L extracellular domain amino acid sequence (human) (SEQ ID NO: 49)
DTQEKEVRAMVGSDVELSCACPEGSRFDLNDVYVYWQTSESKTVVTYHIPQNSSLENVDS
RYRNRALMSPAGMLRGDFSLRLFNVTPQDEQKFHCLVLSQSLGFQEVLSVEVTLHVAANF
SVPVVSAPHSPSQDELTFTCTSINGYPRPNVYWINKTDNSLLDQALQNDTVFLNMRGLYD
VVSVLRIARTPSVNIGCCIENVLLQQNLTVGSQTGNDIGERDKITENPVSTGEKNAATWS
ICOS-L full length cDNA nucleotide sequence (human) (SEQ ID NO: 50)
ACCESSION AF289028

| | |
|---|---|
| 1 | gagtagagcc gatctcccgc gccccgaggt tgctcctctc cgaggtctcc cgcggcccaa |
| 61 | gttctccgcg ccccgaggtc tccgcgcccc gaggtctccg cggcccgagg tctccgcccg |
| 121 | caccatgcgg ctgggcagtc ctggactgct cttcctgctc ttcagcagcc ttcgagctga |
| 181 | tactcaggag aaggaagtca gagcgatggt aggcagcgac gtggagctca gctgcgcttg |
| 241 | ccctgaagga agccgttttg atttaaatga tgtttacgta tattggcaaa ccagtgagtc |
| 301 | gaaaaccgtg gtgacctacc acatcccaca gaacagctcc ttggaaaacg tggacagccg |
| 361 | ctaccggaac cgagccctga tgtcaccggc cggcatgctg cggggcgact tctccctgcg |
| 421 | cttgttcaac gtcacccccc aggacgagca gaagtttcac tgcctggtgt tgagccaatc |
| 481 | cctgggattc caggaggttt tgagcgttga ggttacactg catgtggcag caaacttcag |
| 541 | cgtgcccgtc gtcagcgccc cccacagccc ctcccaggat gagctcacct tcacgtgtac |
| 601 | atccataaac ggctacccca ggcccaacgt gtactggatc aataagacgg acaacagcct |
| 661 | gctggaccag gctctgcaga atgacaccgt cttcttgaac atgcggggct tgtatgacgt |
| 721 | ggtcagcgtg ctgaggatcg cacggacccc cagcgtgaac attggctgct gcatagagaa |
| 781 | cgtgcttctg cagcagaacc tgactgtcgg cagccagaca ggaaatgaca tcggagagag |
| 841 | agacaagatc acagagaatc cagtcagtac cggcgagaaa aacgcggcca cgtggagcat |
| 901 | cctggctgtc ctgtgcctgc ttgtggtcgt ggcggtggcc ataggctggg tgtgcaggga |
| 961 | ccgatgcctc caacacagct atgcaggtgc ctgggctgtg agtccggaga cagagctcac |
| 1021 | tggccacgtt tgaccggagc tcaccgccca gagcgtggac agggcttcca tgagacgcca |
| 1081 | ccgtgagagg ccaggtggca gcttgagcat ggactcccag actgcagggg agcacttggg |
| 1141 | gcagccccca gaaggaccac tgctggatcc cagggagaac ctgctggcgt tggctgtgat |
| 1201 | cctggaatga ggccctttca aaagcgtcat ccacaccaaa ggcaaatgtc cccaagtgag |
| 1261 | tgggctcccc gctgtcactg ccagtcaccc acaggaaggg actggtgatg ggctgtctct |
| 1321 | acccggagcg tgcgggattc agcaccaggc tcttcccagt accccagacc cactgtgggt |
| 1381 | cttcccgtgg gatgcgggat cctgagaccg aagggtgttt ggtttaaaaa gaagactggg |
| 1441 | cgtccgctct tccaggacgg cctctgtgct gctggggtca cgcgaggctg tttgcagggg |
| 1501 | acacggtcac aggagctctt ctgccctgaa cgcttccaac ctgctccggc cggaagccac |
| 1561 | aggacccact ca |

ICOS-L full length amino acid sequence (human) (SEQ ID NO: 51)
ACCESSION O75144
MRLGSPGLLFLLFSSLRADTQEKEVRAMVGSDVELSCACPEGSRFDLNDVYVYVVQTSESK
TVVTYHIPQNSSLENVDSRYRNRALMSPAGMLRGDFSLRLFNVTPQDEQKFHCLVLSQSL
GFQEVLSVEVTLHVAANFSVPVVSAPHSPSQDELTFTCTSINGYPRPNVYWINKTDNSLL
DQALQNDTVFLNMRGLYDVVSVLRIARTPSVNIGCCIENVLLQQNLTVGSQTGNDIGERD
KITENPVSTGEKNAATWSILAVLCLLVVVAVAIGVVVCRDRCLQHSYAGAWAVSPETELTG
HV
ICOS-L extracellular domain cDNA nucleotide sequence (mouse) (SEQ ID NO: 52)
gagactgaagtcggtgcaatggtgggcagcaatgtggtgctcagctgcattgacccccacagacgccatttcaacttgagtggtc
tgtatgtctattggcaaatcgaaaacccagaagtttcggtgacttactacctgccttacaagtctccagggatcaatgtggacagttc
ctacaagaacaggggccatctgtccctggactccatgaagcagggtaacttctctctgtacctgaagaatgtcacccctcaggat
acccaggagttcacatgccgggtatttatgaatacagccacagagttagtcaagatcttggaagaggtggtcaggctgcgtgtgg
cagcaaacttcagtacacctgtcatcagcacctctgatagctccaacccgggccaggaacgtacctacacctgcatgtccaaga
atggctacccagagcccaacctgtattggatcaacacaacggacaatagcctaatagacacggctctgcagaataacactgtct
acttgaacaagttgggcctgtatgatgtaatcagcacattaaggctcccttggacatctcgtggggatgttctgtgctgcgtagagaa
tgtggctctccaccagaacatcactagcattagccaggcagaaagtttcactggaaataacacaaagaacccacaggaaacc
cacaataatgagcttaag
ICOS-L extracellular domain amino acid sequence (mouse) (SEQ ID NO: 53)
ETEVGAMVGSNVVLSCIDPHRRHFNLSGLYVYWQIENPEVSVTYYLPYKSPGINVDSSYK
NRGHLSLDSMKQGNFSLYLKNVTPQDTQEFTCRVFMNTATELVKILEEVVRLRVAANFST
PVISTSDSSNPGQERTYTCMSKNGYPEPNLYWINTTDNSLIDTALQNNTVYLNKLGLYDV
ISTLRLPVVTSRGDVLCCVENVALHQNITSISQAESFTGNNTKNPQETHNNELK
ICOS-L full length amino acid sequence (mouse) (SEQ ID NO: 54)
ACCESSION Q9JHJ8
MQLKCPCFVSLGTRQPVWKKLHVSSGFFSGLGLFLLLLSSLCAASAETEVGAMVGSNVVL
SCIDPHRRHFNLSGLYVYVVQIENPEVSVTYYLPYKSPGINVDSSYKNRGHLSLDSMKQGN
FSLYLKNVTPQDTQEFTCRVFMNTATELVKILEEVVRLRVAANFSTPVISTSDSSNPGQE
RTYTCMSKNGYPEPNLYWINTTDNSLIDTALQNNTVYLNKLGLYDVISTLRLPVVTSRGDV
LCCVENVALHQNITSISQAESFTGNNTKNPQETHNNELKVLVPVLAVLAAAAFVSFIIYR
RTRPHRSYTGPKTVQLELTDHA
ICOS-L full length cDNA nucleotide sequence (mouse) (SEQ ID NO: 55)
ACCESSION AF199027

| | |
|---|---|
| 1 | ccggaacccc aaccgctgca actctccgcg tccgaaatcc agcatcccgc agtctgcgct |
| 61 | cgcaccatgc agctaaagtg tccctgtttt gtgtccttgg gaaccaggca gcctgtttgg |
| 121 | aagaagctcc atgtttctag cgggttcttt tctggtcttg gtctgttctt gctgctgttg |
| 181 | agcagcctct gtgctgcctc tgcagagact gaagtcggtg caatggtggg cagcaatgtg |
| 241 | gtgctcagct gcattgaccc ccacagacgc catttcaact tgagtggtct gtatgtctat |
| 301 | tggcaaatcg aaaacccaga agtttcggtg acttactacc tgccttacaa gtctccaggg |
| 361 | atcaatgtgg acagttccta caagaacagg ggccatctgt ccctggactc catgaagcag |
| 421 | ggtaacttct ctctgtacct gaagaatgtc acccctcagg atacccagga gttcacatgc |
| 481 | cgggtattta tgaatacagc cacagagtta gtcaagatct tggaagaggt ggtcaggctg |
| 541 | cgtgtggcag caaacttcag tacacctgtc atcagcacct ctgatagctc caacccgggc |
| 601 | caggaacgta cctacacctg catgtccaag aatggctacc cagagcccaa cctgtattgg |
| 661 | atcaacacaa cggacaatag cctaatagac acggctctgc agaataacac tgtctacttg |
| 721 | aacaagttgg gcctgtatga tgtaatcagc acattaaggc tcccttggac atctcgtggg |
| 781 | gatgttctgt gctgcgtaga gaatgtggct ctccaccaga acatcactag cattagccag |
| 841 | gcagaaagtt tcactggaaa taacacaaag aacccacagg aaacccacaa taatgagtta |
| 901 | aaagtccttg tccccgtcct tgctgtactg gcggcagcgg cattcgtttc cttcatcata |
| 961 | tacagacgca cgcgtcccca ccgaagctat acaggaccca agactgtaca gcttgaactt |
| 1021 | acagaccacg cctgacagga ctctgcccag gatatggaca gggtttctgt gagttgccac |
| 1081 | caggtggatg tcagacacaa cttcagagtg gacccccaca ggcctggtga cagaggacaa |
| 1141 | cgagctgtct gcttatgggc tgtgatggag gccaggaatc cctggcttta cgaggcacag |
| 1201 | agacttcatc ccagaaaccc cgagggagat ctctccagtg ggcagcagca acatcatcgg |
| 1261 | aatatggagc ctccggtgag ctgtcggcac agagagcagc agcttgtgag aagatccttc |
| 1321 | cttggcacgt tactactcag gcctaggagc tttataaaag agcgtttgag ccactctgaa |
| 1381 | agccctacag agtctactgg agactttccc tgcaggacct tcagttgggg aggaagcctg |
| 1441 | actttattta ggtctcaggc tacttgggcc tcttcgagga tatgtgggat tttgtctact |
| 1501 | gcaaacctgt ttctggctga caatggttgg gctcagaggc actcagcttc acaacatcaa |
| 1561 | tgggacacgc ctcatccttg acttcctgtg gctacagaag ctttccgaaa gccttgagct |
| 1621 | ctttcagact gaacagctct gcccagtctc agcagcccat gaagatctca actccagctt |
| 1681 | cctgggtctc cgtgttgctg gccagaatag agctagctct tttgtttcaa gatggttctg |
| 1741 | caaagttggc tgcttggaaa cctagggatg tatgtacaag ctccaggctg atgcagtagg |
| 1801 | gggcacggac tccccgatgg aacacagtat ctgaccctag gtgagggcaa gctccttccc |
| 1861 | acgcagagga ctggaaattc tggaccgtca aggcctgtct gctatgtggc tggggctcag |
| 1921 | tgctgatgga tgtgtgagat ctcaggaatg aggagtgaga accctgggct caggactagg |
| 1981 | aagacctgtc catttttttt tttttttaat gcccacatgg actttttatt cttcacaccg |
| 2041 | atgtattcaa tgagtgtaga gagaactact taagtccttc ccgagtacaa agcattacct |
| 2101 | acctgcagaa tagcaactgt tgttatgggt cttgagttgg cagctacagc aaacaagcac |
| 2161 | aaggagcagt tggggtgcaa gaagatgggg tgcagcgccc ccaaggacag acatttggga |
| 2221 | attagtggtc tccctgatgc ccatagttcc ccaggaactc aggtgggtct gcggcagcac |
| 2281 | agtaggagta ttcctcctac tttaactttt cttgtcagac gtagtttagg ttcagaaaga |
| 2341 | ggtcaactca gcaagccagc tagccgcctt ggggcaccag acacactgcc ccccaccccc |
| 2401 | tgcttatgta ggcattggga acccttcaca gaccactggc tgtacagtca ccatcacctg |
| 2461 | ctgattccag caggccccca ccttcttgtg gaatcctggg agcactcccc tcttacccct |
| 2521 | cactgccccc caccccctgc acatcagcat tcattagatt tgccctgtaa cgtctgattc |
| 2581 | ctcctttatc tgggttgtag atggggcata gtgacttcta gaaacctaac aagggaataa |
| 2641 | atgtaagatg tgctttcaaa aaaaaaaaaa aaaaaaaaaa aaaaaaaaaa aaaaaaaaaa |
| 2701 | aaaaaaaaaa aaaaaaaa |

Codon Optimized ICOS-L.COMP encodinq nucleotide sequence (human) (SEQ ID NO: 56)

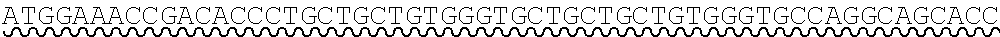

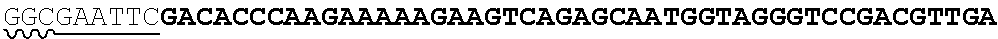

GCTCAGCTGCGCATGTCCCGAAGGGAGCCGATTTGACCTTAACGATGTCTACGTCTA
TTGGCAGACCAGTGAATCAAAGACTGTAGTTACCTATCACATACCACAGAATAGTTCA
TTGGAGAACGTAGATTCACGCTATAGGAATAGAGCTCTGATGTCACCGGCGGGCATG
TTGCGAGGGGACTTTAGCCTGAGGCTTTTCAACGTTACGCCACAAGATGAGCAGAAG
TTCCACTGTCTTGTACTGTCACAGAGTTTGGGATTCCAAGAGGTTCTCTCAGTGGAGG
TCACGTTGCACGTAGCTGCCAATTTCAGTGTCCCTGTTGTCTCTGCACCCCATAGCCC
ATCCCAGGACGAGTTGACATTCACTTGTACAAGCATAAATGGCTACCCACGCCCGAA
TGTCTATTGGATTAACAAGACAGATAATAGCCTCTTGGATCAAGCTCTTCAGAATGAT
ACGGTTTTCCTCAACATGCGCGGGCTTTACGACGTGGTATCCGTTTTGCGAATCGCAC
GAACTCCTTCTGTCAATATCGGTTGCTGCATCGAGAATGTACTCCTGCAGCAGAATCT
TACGGTCGGTTCTCAAACTGGCAATGACATCGGCGAGCGCGATAAAATAACTGAGAA
TCCGGTCAGCACAGGAGAAAAAAACGCCGCAACATGGTCCGAATTCGGCTCTGGACC

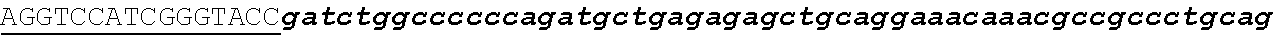

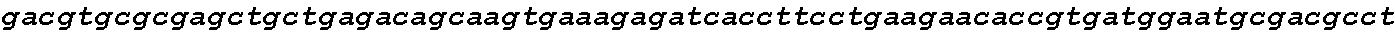

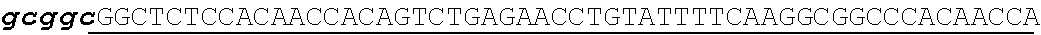

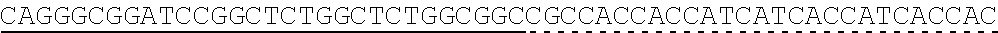

TGATGA
wavy underline = DNA sequencing encoding IgKappa secretion signal
uppercase, no underline (black) = ICOS-L extracellular domain
underlined (gray) = Spacer sequences
lower case italicized = COMP pentamerization domain
stippled underline = DNA sequence encoding a his-tag
double underlined = stop codon
ICOS-L.COMP amino acid sequence (human) (SEQ ID NO: 57)

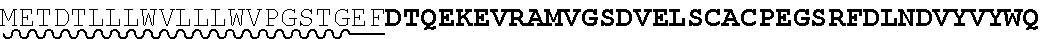

T
SESKTVVTYHIPQNSSLENVDSRYRNRALMSPAGMLRGDFSLRLFNVTPQDEQKFHCLVL
SQSLGFQEVLSVEVTLHVAANFSVPVVSAPHSPSQDELTFTCTSINGYPRPNVYWINKTD
NSLLDQALQNDTVFLNMRGLYDVVSVLRIARTPSVNIGCCIENVLLQQNLTVGSQTGNDI

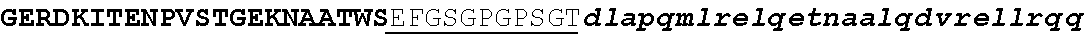

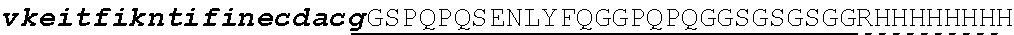

wavy underline = IgKappa secretion signal
Bold (black) = ICOS-L extracellular domain containing IgV domain
lower case italicized = COMP pentamerization domain
stippled underline = Histidine tag
underlined (gray) = Spacer sequences
Codon optimized ICOS-L.COMP nucleotide sequence (mouse) (SEQ ID NO: 58)

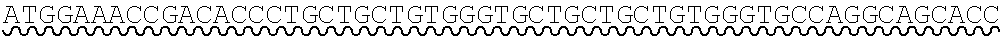

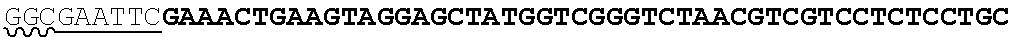

ATTGATCCGCACAGAAGGCATTTCAACTTGTCAGGTCTGTACGTGTACTGGCAAATTG
AAAACCCGGAAGTTTCAGTTACATACTACTTGCCATATAAATCTCCTGGTATAAATGT
AGATAGCTCTTATAAAAATAGAGGACATCTCAGTCTGGATTCAATGAAACAAGGTAAC
TTCTCACTGTACCTTAAGAATGTAACGCCACAAGACACACAGGAATTTACGTGTAGGG
TATTTATGAACACTGCCACAGAACTGGTGAAAATACTTGAAGAGGTTGTGCGCCTGC
GCGTGGCGGCGAACTTTTCAACGCCTGTTATTTCAACTAGTGACAGTTCTAATCCTGG
ACAAGAACGAACGTATACCTGTATGTCCAAGAATGGTTACCCAGAGCCCAACCTTTAT
TGGATAAACACGACCGATAACAGCCTTATTGACACGGCGCTTCAGAACAACACAGTG
TACCTTAACAAATTGGGATTGTATGACGTAATTTCCACGTTGAGACTTCCTTGGACTA
GTAGAGGAGACGTTTTGTGCTGCGTGGAGAATGTTGCTTTGCATCAAAATATTACCTC
AATTTCTCAAGCGGAGTCTTTCACCGGCAATAATACCAAGAACCCACAAGAAACGCA

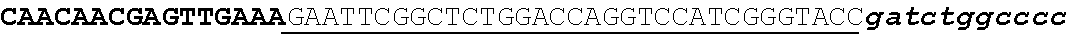

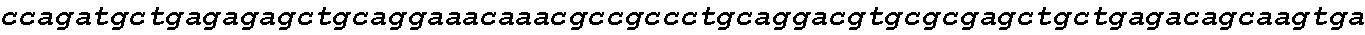

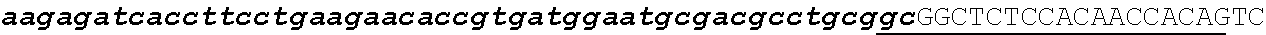

TGAGAACCTGTATTTTCAAGGCGGCCCACAACCACAGGGCGGATCCGGCTCTGGCTCT

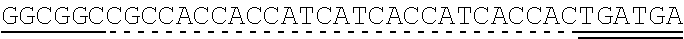

wavy underline = DNA sequencing encoding IgKappa secretion signal
uppercase, no underline (black) = ICOS-L extracellular domain
underlined (gray) = Spacer sequences
lower case italicized = COMP pentamerization domain
stippled underline = DNA sequence encoding a his-tag
double underlined = stop codon
Codon optimized ICOS-L.COMP amino acid sequence (mouse) (SEQ ID NO: 59)

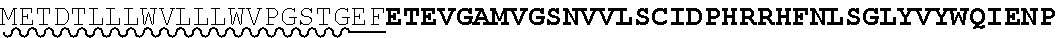

EVSVTYYLPYKSPGINVDSSYKNRGHLSLDSMKQGNFSLYLKNVTPQDTQEFTCRVFMNT
ATELVKILEEVVRLRVAANFSTPVISTSDSSNPGQERTYTCMSKNGYPEPNLYVVINTTDN
SLIDTALQNNTVYLNKLGLYDVISTLRLPWTSRGDVLCCVENVALHQNITSISQAESFTG

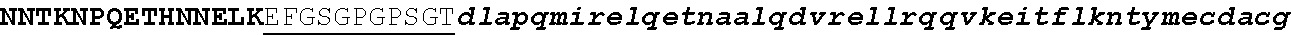

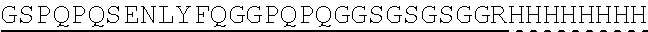

wavy underline = IgKappa secretion signal
Bold (black) = ICOS-L extracellular domain containing IgV domain
lower case italicized = COMP pentamerization domain
stippled underline = Histidine tag
underlined (gray) = Spacer sequences
Strep-tag-II VISTA.COMP amino acid sequence (mouse) (SEQ ID NO: 60)
METDTLLLWVLLLWVPGSTGEFKVTTPYSLYVCPEGQNATLTCRILGPVSKGHDVTIYKTW
YLSSRGEVQMCKEHRPIRNFTLQHLQHHGSHLKANASHDQPQKHGLELASDHHGNFSITL
RNVTPRDSGLYCCLVIELKNHHPEQRFYGSMELQVQAGKGSGSTCMASNEQDSDSITAEF
GSGPGPSGTDLAPQMLRELQETNAALQDVRELLRQQVKEITFLKNTVMECDACGGSPQPQ
SENLYFQGGPQPQGGSGSGSGGLPETGGWSHPQFEK
Human ICOS-L extracellular domain mRNA sequence (SEQ ID NO: 61)

| | |
|---|---|
| 1 | gaguagagcc gaucucccgc gccccgaggu ugcuccucuc cgaggucucc cgcggcccaa |
| 61 | guucuccgcg ccccgagguc uccgcgcccc gaggucuccg cggcccgagg ucuccgcccg |
| 121 | caccaugcgg cugggcaguc cuggacugcu cuuccugcuc uucagcagcc uucgagcuga |
| 181 | uacucaggag aaggaaguca gagcgauggu aggcagcgac guggagcuca gcugcgcuug |
| 241 | cccugaagga agccguuuug auuuaaauga uguuuacgua uauuggcaaa ccagugaguc |
| 301 | gaaaaccgug gugaccuacc acaucccaca gaacagcucc uuggaaaacg uggacagccg |
| 361 | cuaccggaac cgagcccuga ugucaccggc cggcaugcug cggggcgacu ucucccugcg |
| 421 | cuuguucaac gucacccccc aggacgagca gaaguuucac ugccuggugu ugagccaauc |
| 481 | ccugggauuc caggagguuu ugagcguuga gguuacacug cauguggcag caaacuucag |
| 541 | cgugcccguc gucagcgccc cccacagccc cucccaggau gagcucaccu ucacguguac |
| 601 | auccauaaac ggcuacccca ggcccaacgu guacuggauc aauaagacgg acaacagccu |
| 661 | gcuggaccag gcucugcaga augacaccgu cuucuugaac augcggggcu uguaugacgu |
| 721 | ggucagcgug cugaggaucg cacggacccc cagcgugaac auuggcugcu gcauagagaa |
| 781 | cgugcuucug cagcagaacc ugacugucgg cagccagaca ggaaaugaca ucggagagag |
| 841 | agacaagauc acagagaauc cagucaguac cggcgagaaa aacgcggcca cguggagcau |
| 901 | ccuggcuguc cugugccugc uuguggucgu ggcgguggcc auaggcuggg ugugcaggga |
| 961 | ccgaugccuc caacacagcu augcaggugc cugggcugug aguccggaga cagagcucac |
| 1021 | uggccacguu ugaccggagc ucaccgccca gagcguggac agggcuucca ugagacgcca |
| 1081 | ccgugagagg ccagguggca gcuugagcau ggacucccag acugcagggg agcacuuggg |
| 1141 | gcagccccca gaaggaccac ugcuggaucc cagggagaac cugcuggcgu uggcugugau |
| 1201 | ccuggaauga ggcccuuuca aaagcgucau ccacaccaaa ggcaaauguc cccaagugag |
| 1261 | ugggcucccc gcugucacug ccagucaccc acaggaaggg acuggugaug ggcugucucu |
| 1321 | acccggagcg ugcgggauuc agcaccaggc ucuucccagu accccagacc cacugugggu |
| 1381 | cuucccgugg gaugcgggau ccugagaccg aaggguguuu gguuuaaaaa gaagacuggg |
| 1441 | cguccgcucu uccaggacgg ccucugugcu gcugggguca cgcgaggcug uuugcagggg |
| 1501 | acacggucac aggagcucuu cugcccugaa cgcuuccaac cugcuccggc cggaagccac |
| 1561 | aggacccacu ca |

Human PD-L1 extracellular domain mRNA sequence (SEQ ID NO: 62)
uuuacuaucacggcuccaaaggacuuguacgugguggaguauggcagcaacgucacgauggagugcagauucccu
guagaacgggagcuggaccugcuugcguuagugguguacugggaaaaggaagaugagcaagugauucaguuugu
ggcaggagaggaggaccuuaagccucagcacagcaacuucagggggagagccucgcugccaaaggaccagcuuuu
gaagggaaaugcugcccuucagaucacagacgucaagcugcaggacgcaggcguuuacugcugcauaaucagcua
cgguggugcggacuacaagcgaaucacgcugaaagucaaugccccauaccgcaaaaucaaccagagaauuuccgu
ggauccagccacuucugagcaugaacuaauaugucaggccgaggguuauccagaagcugagguaaucuggacaaa
cagugaccaccaacccgugagugggaagagaagugucaccacuucccggacagaggggaugcuucucaaugugac
cagcagucugagggucaacgccacagcgaaugauguuuucuacuguacguuuuggagaucacagccagggcaaaa
ccacacagcggagcugaucaucccagaacugccugcaacacauccuccacagaacagg

### REFERENCES CITED

1. Topalian S L, Hodi F S, Brahmer J R, et al. Safety, activity, and immune correlates of anti-PD-1 antibody in cancer. N Engl J Med. 2012; 366(26):2443-2454.
2. Hodi F S, O'Day S J, McDermott D F, et al. Improved survival with ipilimumab in patients with metastatic melanoma. N Engl J Med. 2010; 363(8):711-723.
3. Hodi F S, Chesney J, Pavlick A C, et al. Combined nivolumab and ipilimumab versus ipilimumab alone in patients with advanced melanoma: 2-year overall survival outcomes in a multicentre, randomised, controlled, phase 2 trial. Lancet Oncol. 2016; 17(11):1558-1568.
4. Francisco L M, Sage P T, Sharpe A H. The PD-1 pathway in tolerance and autoimmunity. Immunol Rev. 2010; 236:219-242.
5. Raptopoulou A P, Bertsias G, Makrygiannakis D, et al. The programmed death 1/programmed death ligand 1 inhibitory pathway is up-regulated in rheumatoid synovium and regulates peripheral T cell responses in human and murine arthritis. Arthritis Rheum. 2010; 62(7):1870-1880.
6. Riella L V, Paterson A M, Sharpe A H, Chandraker A. Role of the PD-1 pathway in the immune response. Am J Transplant. 2012; 12(10):2575-2587.
7. Sharpe A H, Freeman G J. The B7-CD28 superfamily. Nat Rev Immunol. 2002; 2(2):116-126.
8. Lines J L, Pantazi E, Mak J, et al. VISTA is an immune checkpoint molecule for human T cells. Cancer Res. 2014; 74(7):1924-1932.
9. Wang L, Rubinstein R, Lines J L, et al. VISTA, a novel mouse Ig superfamily ligand that negatively regulates T cell responses. J Exp Med. 2011; 208(3):577-592.
10. Flies D B, Han X, Higuchi T, et al. Coinhibitory receptor PD-1H preferentially suppresses CD4+ T cell-mediated immunity. J Clin Invest. 2014; 124(5):1966-1975.
11. Lines J L, Sempere L F, Broughton T, Wang L, Noelle R. VISTA is a novel broad-spectrum negative checkpoint regulator for cancer immunotherapy. Cancer Immunol Res. 2014; 2(6):510-517.
12. Le Mercier I, Chen W, Lines J L, et al. VISTA Regulates the Development of Protective Antitumor Immunity. Cancer Res. 2014; 74(7):1933-1944.
13. Ceeraz S, Sergent P A, Plummer S F, et al. VISTA deficiency accelerates the development of fatal murine lupus nephritis. Arthritis Rheumatol. 2016.
14. Wang L, Le Mercier I, Putra J, et al. Disruption of the immune-checkpoint VISTA gene imparts a proinflammatory phenotype with predisposition to the development of autoimmunity. Proc Natl Acad Sci USA. 2014; 111(41):14846-14851.
15. Yoon K W, Byun S, Kwon E, et al. Control of signaling-mediated clearance of apoptotic cells by the tumor suppressor p53. Science. 2015; 349(6247):1261669.
16. Flies D B, Higuchi T, Chen L. Mechanistic Assessment of PD-1H Coinhibitory Receptor-Induced T Cell Tolerance to Allogeneic Antigens. J Immunol. 2015; 194(11):5294-5304.
17. Freeman G J, Long A J, Iwai Y, et al. Engagement of the PD-1 immunoinhibitory receptor by a novel B7 family member leads to negative regulation of lymphocyte activation. J Exp Med. 2000; 192(7): 1027-1034.
18. Terawaki S, Chikuma S, Shibayama S, et al. IFN-α directly promotes programmed cell death-1 transcription and limits the duration of T cell-mediated immunity. J Immunol. 2011; 186(5):2772-2779.
19. Sanmamed M F, Chen L. Inducible expression of B7-H1 (PD-L1) and its selective role in tumor site immune modulation. Cancer J. 2014; 20(4):256-261.
20. Latchman Y, Wood C R, Chernova T, et al. PD-L2 is a second ligand for PD-1 and inhibits T cell activation. Nat Immunol. 2001; 2(3):261-268.
21. Wang G, Hu P, Yang J, Shen G, Wu X. The effects of PDL-Ig on collagen-induced arthritis. Rheumatol Int. 2011; 31(4):513-519.
22. Sica G L, Choi I H, Zhu G, et al. B7-H4, a molecule of the B7 family, negatively regulates T cell immunity. Immunity. 2003; 18(6):849-861.
23. Xu J F, Xiao H, Hu G Y, et al. Ectopic B7-H4-Ig expression attenuates concanavalin A-induced hepatic injury. Clin Immunol. 2010; 136(1):30-41.
24. Azuma T, Zhu G, Xu H, et al. Potential role of decoy B7-H4 in the pathogenesis of rheumatoid arthritis: a mouse model informed by clinical data. PLoS Med. 2009; 6(10):e1000166.
25. Hutloff A, Dittrich A M, Beier K C, et al. ICOS is an inducible T-cell co-stimulator structurally and functionally related to CD28. Nature. 1999; 397(6716):263-266.
26. Zhang X, Schwartz J C, Guo X, et al. Structural and functional analysis of the costimulatory receptor programmed death-1. Immunity. 2004; 20(3):337-347.
27. Buonfiglio D, Bragardo M, Redoglia V, et al. The T cell activation molecule H4 and the CD28-like molecule ICOS are identical. Eur J Immunol. 2000; 30(12):3463-3467.
28. Beier K C, Hutloff A, Dittrich A M, et al. Induction, binding specificity and function of human ICOS. Eur J Immunol. 2000; 30(12):3707-3717.
29. Mages H W, Hutloff A, Heuck C, et al. Molecular cloning and characterization of murine ICOS and identification of B7h as ICOS ligand. Eur J Immunol. 2000; 30(4):1040-1047.
30. Yoshinaga S K, Zhang M, Pistillo J, et al. Characterization of a new human B7-related protein: B7RP-1 is the ligand to the co-stimulatory protein ICOS. Int Immunol. 2000; 12(10):1439-1447.
31. Yoshinaga S K, Whoriskey J S, Khare S D, et al. T-cell co-stimulation through B7RP-1 and ICOS. Nature. 1999; 402(6763):827-832.
32. Wang S, Zhu G, Chapoval A I, et al. Costimulation of T cells by B7-H2, a B7-like molecule that binds ICOS. Blood. 2000; 96(8):2808-2813.
33. Arimura Y, Kato H, Dianzani U, et al. A co-stimulatory molecule on activated T cells, H4/ICOS, delivers specific signals in T(h) cells and regulates their responses. Int Immunol. 2002; 14(6):555-566.
34. Gigoux M, Shang J, Pak Y, et al. Inducible costimulator promotes helper T-cell differentiation through phosphoinositide 3-kinase. Proc Natl Acad Sci USA. 2009; 106(48):20371-20376.
35. Fan X, Quezada S A, Sepulveda M A, Sharma P, Allison J P. Engagement of the ICOS pathway markedly enhances efficacy of CTLA-4 blockade in cancer immunotherapy. J Exp Med. 2014; 211(4):715-725.
36. Chen H, Liakou C I, Kamat A, et al. Anti-CTLA-4 therapy results in higher CD4+ICOShi T cell frequency and IFN-gamma levels in both nonmalignant and malignant prostate tissues. Proc Natl Acad Sci USA. 2009; 106(8):2729-2734.
37. Liakou C I, Kamat A, Tang D N, et al. CTLA-4 blockade increases IFNgamma-producing CD4+ICOShi cells to shift the ratio of effector to regulatory T cells in cancer patients. Proc Natl Acad Sci USA. 2008; 105(39):14987-14992.
38. Chen X, Zaro J L, Shen W C. Fusion protein linkers: property, design and functionality. Adv Drug Deliv Rev. 2013; 65(10):1357-1369.
39. Prodeus A, Cydzik M, Abdul-Wahid A, et al. Agonistic CD200R1 DNA Aptamers Are Potent Immunosuppressants That Prolong Allogeneic Skin Graft Survival. Mol Ther Nucleic Acids. 2014; 3:e190.
40. Lin D Y, Tanaka Y, Iwasaki M, et al. The PD-1/PD-L1 complex resembles the antigen-binding Fv domains of antibodies and T cell receptors. Proc Natl Acad Sci USA. 2008; 105(8):3011-3016.
41. van der Merwe P A, Bodian D L, Daenke S, Linsley P, Davis S J. CD80 (B7-1) binds both CD28 and CTLA-4 with a low affinity and very fast kinetics. J Exp Med. 1997; 185(3):393-403.
42. Haughn L, Gratton S, Caron L, Sékaly R P, Veillette A, Julius M. Association of tyrosine kinase p56Ick with CD4 inhibits the induction of growth through the alpha beta T-cell receptor. Nature. 1992; 358(6384):328-331.
43. Tiegs G, Hentschel J, Wendel A. A T cell-dependent experimental liver injury in mice inducible by concanavalin A. J Clin Invest. 1992; 90(1):196-203.

1-51. (canceled)

52. A pentamerized polypeptide having T-cell inhibitory activity comprising:
five monomers, each of the monomers comprising:
a polypeptide having substantial similarity to the extracellular domain of a V-domain Ig Suppressor of T cell Activation (VISTA) (SEQ ID NO: 9) linked to a polypeptide having substantial similarity to a pentamerization domain of cartilage oligomeric matrix protein (COMP) (SEQ ID NO: 11).

53. The pentamerized polypeptide of claim 52, wherein the pentamerized polypeptide is in a soluble form.

54. The pentamerized polypeptide of claim 53, wherein the soluble form pentamerized polypeptide has increased T-cell inhibitory activity relative to a soluble dimerized polypeptide comprising the extracellular domain of VISTA (SEQ ID NO: 9).

55. The pentamerized polypeptide of claim 54, wherein the increased T-cell inhibitory activity comprises one or more of increased inhibition of T-cell activation and T-cell proliferation.

56. The pentamerized polypeptide of claim 53, wherein the soluble form pentamerized polypeptide has increased immune inhibitory activity in vivo relative to a soluble dimerized polypeptide comprising the extracellular domain of VISTA (SEQ ID NO: 9).

57. The pentamerized polypeptide of claim 56, wherein the increased immune inhibitory activity comprises one or more of increased inhibition of cytokine secretion and cytotoxic lymphocyte (CTL) production.

58. A recombinant polypeptide comprising:
a polypeptide having substantial similarity to the extracellular domain of a V-domain Ig Suppressor of T cell Activation (VISTA) (SEQ ID NO: 9) linked to a polypeptide having substantial similarity to a pentamerization domain of cartilage oligomeric matrix protein (COMP) (SEQ ID NO: 11).

59. The recombinant polypeptide of claim 58, wherein the recombinant polypeptide is in a soluble form.

60. A recombinant nucleic acid comprising:
a nucleic acid having substantial similarity to a nucleic acid encoding the extracellular IgV containing domain of a V-domain Ig Suppressor of T cell Activation (VISTA) polypeptide, the nucleic acid having a sequence of SEQ ID NO: 1; and
a nucleic acid having substantial similarity to a nucleic acid encoding a pentamerization domain of cartilage oligomeric matrix protein (COMP) having a sequence of SEQ ID NO: 3, the nucleic acid encoding the extracellular IgV containing domain of the VISTA polypeptide being operably linked to the nucleic acid encoding the pentamerization domain of COMP.

61. An expression vector comprising the recombinant nucleic acid of claim 60.

62. The expression vector of claim 61, further comprising at least one control sequence.

63. A host cell comprising the expression vector of claim 61 or 62.

64. A pharmaceutical composition comprising one or more of:
the polypeptide of any one of claims 58-59, the host cell of claim 63, and the pentamerized polypeptide of anyone of claims 51-57; and a pharmaceutically acceptable carrier, diluent, or excipient.

65. A method of eliciting a biological response in an individual in need thereof, the method comprising: administering to the individual a therapeutically effective amount of a V-domain Ig Suppressor of T cell Activation (VISTA)-cartilage oligomeric matrix protein (COMP) fusion polypeptide (VISTA.COMP), the VISTA.COMP polypeptide having SEQ ID NO: 9 and being linked to SEQ ID NO: 11, wherein the biological response is one or more of:
suppression of T-cell activation;
suppression of T-cell proliferation;
decreased secretion by T-cells of one or more inflammatory cytokines;
suppressed induction of cytotoxic T lymphocytes (CTLs); and
an increase in T-cells with regulatory phenotypes.

66. The method of claim 65, wherein the inflammatory cytokines comprise one or more of IL-2 and IFNγ.

67. A pentamerized polypeptide having T-cell inhibitory activity comprising:
five monomers, each of the monomers comprising:
a polypeptide having substantial similarity to the extracellular domain of B7-H4 (SEQ ID NO: 25) linked to a polypeptide having substantial similarity to a pentamerization domain of cartilage oligomeric matrix protein (COMP) (SEQ ID NO: 11).

68. The pentamerized polypeptide of claim 67, wherein the pentamerized polypeptide is in a soluble form.

69. The pentamerized polypeptide of claim 67, wherein the pentamerized polypeptide has increased T-cell inhibitory activity.

70. A recombinant polypeptide comprising:
a polypeptide having substantial similarity to the extracellular domain of B7-H4 (SEQ ID NO: 25) linked to a polypeptide having substantial similarity to a pentamerization domain of cartilage oligomeric matrix protein (COMP) (SEQ ID NO: 11).

71. The recombinant polypeptide of claim 70, wherein the recombinant polypeptide is in a soluble form.

72. A recombinant nucleic acid comprising:
a nucleic acid having substantial similarity to a nucleic acid encoding the extracellular domain of B7-H4 polypeptide, the nucleic acid having a sequence of SEQ ID NO: 26; and
a nucleic acid having substantial similarity to a nucleic acid encoding a pentamerization domain of cartilage oligomeric matrix protein (COMP) having a sequence of SEQ ID NO: 3, the nucleic acid encoding the extracellular domain of B7-H4 polypeptide being operably linked to the nucleic acid encoding the pentamerization domain of COMP.

73. An expression vector comprising the recombinant nucleic acid of claim 72.

74. The expression vector of claim 73, further comprising at least one control sequence.

75. A host cell comprising the expression vector of claim 73 or 74.

76. A pharmaceutical composition comprising one or more of:
the polypeptide of any one of claims 70-71, the host cell of claim 75, and the pentamerized polypeptide of anyone of claims 67-69; and a pharmaceutically acceptable carrier, diluent, or excipient.

77. A method of eliciting a biological response in an individual in need thereof, the method comprising: administering to the individual a therapeutically effective amount of a B7-H4-cartilage oligomeric matrix protein (COMP) fusion polypeptide (B7-H4.COMP), the B7-H4.COMP polypeptide having SEQ ID NO: 25 and being linked to SEQ ID NO: 11, wherein the biological response is one or more of:
suppression of T-cell activation;
suppression of T-cell proliferation;
decreased secretion by T-cells of one or more inflammatory cytokines;
suppressed induction of cytotoxic T lymphocytes (CTLs); and
an increase in T-cells with regulatory phenotypes.

78. A pentamerized polypeptide having T-cell inhibitory activity comprising:
five monomers, each of the monomers comprising:
a polypeptide having substantial similarity to the extracellular domain of PD-L1 (SEQ ID NO: 36) linked to a polypeptide having substantial similarity to a pentamerization domain of cartilage oligomeric matrix protein (COMP) (SEQ ID NO: 11).

79. The pentamerized polypeptide of claim 78, wherein the pentamerized polypeptide is in a soluble form.

80. The pentamerized polypeptide of claim 78, wherein the pentamerized polypeptide has increased T-cell inhibitory activity.

81. A recombinant polypeptide comprising:
a polypeptide having substantial similarity to the extracellular domain of PD-L1 (SEQ ID NO: 36) linked to a polypeptide having substantial similarity to a pentamerization domain of cartilage oligomeric matrix protein (COMP) (SEQ ID NO: 11).

82. The recombinant polypeptide of claim 81, wherein the recombinant polypeptide is in a soluble form.

83. A recombinant nucleic acid comprising:
a nucleic acid having substantial similarity to a nucleic acid encoding the extracellular domain of PD-L1 polypeptide, the nucleic acid having a sequence of SEQ ID NO: 37; and
a nucleic acid having substantial similarity to a nucleic acid encoding a pentamerization domain of cartilage oligomeric matrix protein (COMP) having a sequence of SEQ ID NO: 3, the nucleic acid encoding the extracellular domain of PD-L1 polypeptide being operably linked to the nucleic acid encoding the pentamerization domain of COMP.

84. An expression vector comprising the recombinant nucleic acid of claim 83.

85. The expression vector of claim 84, further comprising at least one control sequence.

86. A host cell comprising the expression vector of claim 84 or 85.

87. A pharmaceutical composition comprising one or more of:
the polypeptide of any one of claims 81-82, the host cell of claim 86, and the pentamerized polypeptide of anyone of claims 78-80; and a pharmaceutically acceptable carrier, diluent, or excipient.

88. A method of eliciting a biological response in an individual in need thereof, the method comprising: administering to the individual a therapeutically effective amount of a PD-L1-cartilage oligomeric matrix protein (COMP) fusion polypeptide (PD-L1.COMP), the PD-L1.COMP polypeptide having SEQ ID NO: 36 and being linked to SEQ ID NO: 11, wherein the biological response is one or more of:
suppression of T-cell activation;
suppression of T-cell proliferation;
decreased secretion by T-cells of one or more inflammatory cytokines;
suppressed induction of cytotoxic T lymphocytes (CTLs); and
an increase in T-cells with regulatory phenotypes.
